(19) United States
(12) Patent Application Publication
(10) Pub. No.: US 2026/0364216 A1
(43) Pub. Date: Nov. 19, 2026

(54) Combination Therapy

(71) Applicant: Telix Pharmaceuticals (Innovations) Pty Ltd, North Melbourne (AU)

(72) Inventors: Kwame Twumasi-Boateng, North Melbourne (AU); Sandra Heskamp, Nijmegen (NL); Simone Caroline Kleinendorst, Nijmegen (NL); Michael Paul Wheatcroft, North Melbourne (AU)

(21) Appl. No.: 18/863,804

(22) PCT Filed: Jun. 9, 2023
(86) PCT No.: PCT/AU2023/050509

§ 371 Date: Dec. 17, 2025

(30) Foreign Application Priority Data
Jun. 9, 2022 (AU) 2022901582
Oct. 11, 2022 (AU) 2022902973

(51) Int. Cl. A61K 51/10; A61K 35/00; A61K 39/00; C07K 16/28; C07K 16/30

(52) U.S. Cl. CPC A61K 51/1093; A61K 35/00; A61K 51/1045; C07K 16/2818; C07K 16/30; A61K 2039/505; A61K 2039/545; A61K 2121/00; C07K 2317/52; C07K 2317/565; C07K 2317/567; C07K 2317/71; C07K 2317/94

(57) ABSTRACT

The invention relates to methods and compositions for use in combination radioimmunotherapy.

### FIELD OF THE INVENTION

The invention relates to combination therapies of checkpoint inhibitors and radiolabelled agents for binding to CAIX, in particular agents such as antibodies conjugated to a radioisotope for use in radioimmmunotherapy, and kits and compositions for use in the same.

### RELATED APPLICATIONS

This application claims priority from Australian provisional applications AU 2022901582 and AU 2022902973, the entire contents of which are hereby incorporated by reference.

### BACKGROUND OF THE INVENTION

Radiotherapy is an important form of tumor therapy. Various methods of radiotherapy have been developed to treat tumors. Among them, radioimmunotherapy (RAIT or RIT), also termed molecular targeted radiation (MTR) or targeted radionuclide therapy (TRT), is one emerging approach to the provision of radiotherapy. It employs antibodies, antibody fragments or peptides capable of binding to target cells, in order to direct radioisotopes to specific tissues and cells, thus enhancing specificity of tumor treatment and reducing toxicity.

Radiation damage to healthy tissues and organs is a major problem associated with radiotherapy. Such damage has been primarily attributed to radiation-generated reactive oxygen species which oxidize functionally important biological molecules, such as nucleic acids, carbohydrates, lipids and lipoproteins, and damage tissues and cells. They have been implicated in a variety of biological processes, e.g., antimicrobial defense, inflammation, carcinogenesis and aging. As reflected by body weight loss, myelosuppression and blood cell loss, such as decreased white blood cell (WBC) and platelet counts and hematopoietic toxicity are the most notable consequences of the radiation damage. The toxicity severely limits the radiation dosage of RAIT and reduces the effectiveness of tumor treatment.

A number of methods have been developed to attempt to mitigate the hematopoietic toxicity of radiation. Stem cell transplantation (SCT) and bone marrow transplantation (BMT) are the most frequently used methods. However, these approaches are invasive, expensive and may contribute to longer hospitalization of the individual receiving treatment.

Other methods include using cytokines to stimulate the immune system and hemoregulatory proteins such as HP5b to turn off hematopoiesis during the radiation exposure period. These methods have achieved various degrees of success in combating hematopoietic toxicity in small studies but again, require exposing the patient to further medication and treatment, therefore remain largely unused.

Immunotherapies have shown promise for the treatment of cancer due to their ability to slow the growth and spread of cancer cells, and by helping the immune system destroy existing cancer cells. Immunotherapies may assist the immune system by priming and boosting the immune system via stimulation of antigen-presenting cells, T-cells, or innate cells, by reducing immunosuppression in the tumor environment by regulating inhibitory pathways, for example by enhancing adaptive or innate immune responses.

An example of a class of immunotherapeutics are the checkpoint inhibitors. Checkpoint inhibitors currently approved by the US Food and Drug Administration (FDA) target the cytotoxic T-lymphocyte-associated protein 4 (CTLA-4), programmed cell death receptor 1 (PD-1), programmed cell death ligand 1 (PD-L1) or LAG3. Such checkpoint inhibitors work by blocking T cell-inhibition signals. The first approved agent, ipilimumab, received FDA marketing authorization in 2011 for metastatic melanoma. Since ipilimumab, at least five more checkpoint inhibitor drugs have been approved for at least 14 different indications.

Immune checkpoint inhibition (ICI) has substantially changed cancer treatment, but (long-lasting) responses remain absent in the majority of patients. Critical determinants for successful ICI treatment are high tumor mutational burden and pre-existing tumor infiltrating lymphocytes (TIL).

There remains a need for new methods for mitigating the toxicity associated with radioimmunotherapy.

There also remains a need for improved immunotherapies for the treatment of cancer.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides combined immune checkpoint inhibition (ICI) and targeted radionuclide therapy (TRT). Surprisingly, combined ICI and TRT showed improved outcomes relative to either therapy type alone, allowing for lower TRT doses and in certain embodiments, immunological memory of the subject tumor.

Accordingly, the present invention provides a method of treating, preventing or minimising progression of cancer in a subject, the method comprising:
administering to the subject, in combination with immune checkpoint inhibitor therapy, a radiolabelled agent that binds to or specifically binds to carbonic anhydrase IX (CAIX),
thereby treating, preventing or minimising progression of cancer in the subject.

In another aspect, the present invention provides a method of treating, preventing or minimising progression of cancer in a subject, the method comprising:
administering to the subject a radiolabelled agent that binds to or specifically binds to carbonic anhydrase IX (CAIX), and
administering to the subject an immune checkpoint inhibitor, thereby treating, preventing or minimising progression of cancer in the subject.

In a further aspect, the present invention provides a method of treating, preventing or minimising the progression of cancer in a subject who has received, or who is receiving checkpoint inhibitor therapy, the method comprising:
administering to the subject a radiolabelled agent that binds to or specifically binds to carbonic anhydrase IX (CAIX),
thereby treating, preventing or minimising progression of cancer in the subject.

In any aspect or embodiment, the radiolabelled agent may be a small molecule, a peptide, or a protein, conjugated to a radionuclide, and which is capable of binding to CAIX. Preferably the agent is capable of specifically and/or selectively binding to CAIX.

In any embodiment, the agent for binding to CAIX is a small molecule, optionally selected from the group consisting of: SLC-0111, SLC-149, SLC-0121, SLC-101, PMI-05, sulfamide-nitroimidazole, JS-403, UB-TT220, HEHEHE-Z09781, -MIP-1486, MIP-1490, MIP-1504 (especially ^{99m}Tc-HEHEHE-Z09781, ^{99m}Tc-MIP-1486, ^{99m}Tc-MIP-1490 or ^{99m}Tc-MIP-1504/5) and PHC-102.

In any embodiment, the agent for binding to CAIX is a peptide, optionally selected from the group consisting of: 3B-301, 3B-302 or CAIX-P1.

In especially preferred aspects and embodiments, the agent for binding to CAIX is a polypeptide, including an antibody or antigen-binding fragment thereof.

In one aspect, the agent is a peptide or protein, such that the present invention provides a method of treating, preventing or minimising progression of cancer in a subject, the method comprising:
administering to the subject, in combination with immune checkpoint inhibitor therapy, a radiolabelled peptide or radioconjugated protein, that binds to or specifically binds to carbonic anhydrase IX (CAIX),
thereby treating, preventing or minimising progression of cancer in the subject.

In another aspect, the present invention provides a method of treating, preventing or minimising progression of cancer in a subject, the method comprising:
administering to the subject a radiolabelled peptide or radioconjugated protein that binds to or specifically binds to carbonic anhydrase IX (CAIX), and administering to the subject an immune checkpoint inhibitor,
thereby treating, preventing or minimising progression of cancer in the subject.

In another aspect, the agent is a radiolabelled antibody or antigen binding fragment thereof, such that the present invention provides a method of treating, preventing or minimising progression of cancer in a subject, the method comprising:
administering to the subject, in combination with immune checkpoint inhibitor therapy, an antibody or antigen binding fragment thereof for binding to CAIX, wherein the antibody or antigen binding fragment thereof is conjugated to a radionuclide,
   thereby treating, preventing or minimising progression of cancer in the subject.

In another aspect, the present invention provides a method of treating, preventing or minimising the progression of cancer in a subject who has received, or who is receiving a checkpoint inhibitor, the method comprising:
administering to the subject an antigen binding protein that binds to or specifically binds to carbonic anhydrase IX (CAIX), wherein the antigen binding protein is conjugated to a radionuclide,
thereby treating, preventing or minimising progression of cancer in the subject.

In another aspect, the present invention provides a method of treating, preventing or minimising the progression of cancer in a subject who has received, or who is receiving a checkpoint inhibitor, the method comprising:
administering to the subject an antibody or antigen binding fragment thereof for binding to CAIX, wherein the antibody or antigen binding fragment thereof is conjugated to a radionuclide,
thereby treating, preventing or minimising progression of cancer in the subject.

In any aspect of the invention, the radiolabelled agent (preferably a radiolabelled antigen binding protein such as an antibody) that binds to or specifically binds to carbonic anhydrase IX (CAIX) and the checkpoint inhibitor, may be administered at the same time. Alternatively, they may be administered sequentially. For instance, the checkpoint inhibitor may be administered prior to the radiolabelled agent (preferably a radiolabelled antigen binding protein) or the radiolabelled agent (preferably a radiolabelled antigen binding protein) may be administered prior to the checkpoint inhibitor. Alternatively, treatment with the checkpoint inhibitor and/or the radiolabelled agent (preferably a radiolabelled antigen binding protein) may be staggered.

In any aspect or embodiment, the immune checkpoint inhibitor therapy targets any one or more of: PD-1, PD-L1 and CTLA-4, and is a PD-1, PD-L1, and/or CTLA-4 checkpoint inhibitor. The checkpoint inhibitor may be an antibody or antigen binding fragment thereof, a protein, a peptide or a small molecule. In any aspect or embodiment, the checkpoint inhibitor is an inhibitor of PD-1, PD-L1, or CTLA-4 in the form of an antibody or antigen binding fragment thereof. In any aspect or embodiment, the checkpoint inhibitor is an inhibitor of PD-1, PD-L1, or CTLA-4 in the form of a peptide. In any aspect or embodiment, the checkpoint inhibitor is an inhibitor of PD-1. In any aspect or embodiment, the checkpoint inhibitor is an inhibitor of CTLA-4. In any aspect or embodiment, immune checkpoint inhibitor therapy involves administering inhibitors of PD-1 and CTLA-4. In any aspect or embodiment, immune checkpoint inhibitor therapy involves administering inhibitors of PD-L1 and CTLA-4.

In any aspect or embodiment described herein, the agent that binds to or specifically binds to carbonic anhydrase IX (CAIX) may be an antibody that binds to or specifically binds to CAIX, or an antigen binding fragment thereof. In some embodiments, the antibody is girentuximab or a variant or antigen binding fragment thereof that retains the ability to bind to CAIX.

The radiolabelled agent (preferably a radiolabelled antibody or fragment thereof) may be conjugated to any radionuclide suitable for therapeutic use, including any of: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 and radium-224 (²²³Ra, ²²⁴Ra), rhenium-186 and rhenium-188 (¹⁸⁶Re and ¹⁸⁸Re), samarium-153 (¹⁵³Sm), scandium-47 (⁴⁷Sc), strontium-90 (⁹⁰Sr), terbium-149 and terbium-161 (¹⁴⁹Tb and ¹⁶¹Tb), zirconium (⁸⁹Zr) and yttrium-90 (⁹⁰Y). In some embodiments, the agent is an antibody or antigen binding fragment thereof and the radionuclide conjugated to the antibody or fragment thereof is lutetium-¹⁷⁷.

As used herein the terms radioconjugated and radiolabelled may be used interchangeably. The terms will be understood to refer to an agent (such as a small molecule, peptide, or polypeptide such as an antibody or antigen binding fragment thereof), that is conjugated to a radionuclide. It will also be appreciated that the terms radionuclide and radioisotope may be used herein interchangeably.

In another aspect, the present invention provides a method of inducing an immune response, preferably a T-cell immune response, to a cancer in a subject, comprising administering to the subject a radiolabelled agent for binding to CAIX, in combination with an immune checkpoint inhibitor, thereby inducing the T cell response to the cancer in the subject. Preferably the radiolabelled agent is a radiolabelled peptide or a radiolabelled antibody or antigen binding fragment thereof.

In another aspect, the present invention provides a method of inducing an adaptive immune response to a cancer in a subject, comprising administering to the subject, a radiolabelled agent that binds to or specifically binds to CAIX, in combination with an immune checkpoint inhibitor, thereby inducing a T cell response to the cancer in the subject. Preferably the radiolabelled agent is a radiolabelled peptide or a radiolabelled antibody or antigen binding fragment thereof.

In any aspect or embodiment of the invention, the methods described herein further comprise identifying a subject having cancer. In an embodiment, the cancer may be pre-cancerous or non-metastatic. In another embodiment, the cancer may be malignant or metastatic.

In another aspect, the present invention provides a method of treating, preventing or minimising progression of cancer in a subject comprising the steps of:
identifying a subject having cancer who has received, or who is receiving a checkpoint inhibitor for the treatment of cancer,
assessing whether the subject is responsive to the checkpoint inhibitor,
   wherein if the subject is not responsive to the checkpoint inhibitor:
administering to the subject, a radiolabelled agent (preferably a radiolabelled antigen binding protein) that binds to or specifically binds to CAIX,
thereby treating, preventing or minimising progression of cancer in the subject.

In another aspect, the present invention provides a method of treating, preventing or minimising progression of cancer in a subject comprising the steps of:
identifying a subject having cancer and being unresponsive to a treatment comprising a checkpoint inhibitor,
administering to the subject, a radiolabelled agent comprising, consisting or consisting essentially of a radiolabelled antigen binding protein that binds to or specifically binds to CAIX,
thereby treating, preventing or minimising progression of cancer in the subject.

In another aspect, the present invention provides a method of treating, preventing or minimising progression of cancer in a subject comprising the steps of:
identifying a subject having cancer; and
administering to the subject a radiolabelled agent (preferably a radiolabelled antigen binding protein) that binds to or specifically binds to CAIX,] and a checkpoint inhibitor,
thereby treating, preventing or minimising progression of cancer in the subject.

In another aspect, the present invention provides a method of treating, preventing or minimising progression of cancer in a subject comprising the steps of:
identifying a subject having cancer; and
administering to the subject, a radiolabelled agent (preferably a radiolabelled antibody or antigen binding fragment thereof), for binding to CAIX, and a checkpoint inhibitor,
thereby treating, preventing or minimising progression of cancer in the subject.

In another aspect, the present invention further provides a method of increasing survival of a subject having cancer comprising administering to the subject, a radiolabelled agent (preferably a radiolabelled antigen binding protein) that binds to or specifically binds to CAIX, and a checkpoint inhibitor, thereby increasing survival of the subject having cancer.

In another aspect, the present invention further provides a method of increasing survival of a subject having cancer comprising administering to the subject, a radiolabelled agent (preferably a radiolabelled antibody or antigen binding fragment thereof) for binding to CAIX, and a checkpoint inhibitor, thereby increasing survival of the subject having cancer.

In another aspect, the present invention further provides a method of minimising, reducing or preventing growth of a tumor in a subject having cancer comprising administering to the subject a radiolabelled agent (preferably a radiolabelled antigen binding protein) that binds to or specifically binds to CAIX, in combination with immune checkpoint inhibitor therapy, thereby minimising, reducing or preventing growth of a tumor in the subject having cancer.

In another aspect, the present invention further provides a method of minimising, reducing or preventing growth of a tumor in a subject having cancer comprising administering to the subject a radiolabelled agent (preferably a radiolabelled antibody or antigen binding fragment thereof) for binding to CAIX, in combination with immune checkpoint inhibitor therapy, thereby minimising, reducing or preventing growth of a tumor in the subject having cancer.

In another aspect, the present invention further provides a method of minimising, reducing or preventing metastasis in a subject having cancer comprising administering to the subject of a radiolabelled agent (preferably a radiolabelled antigen binding protein) that binds to or specifically binds to CAIX, in combination with immune checkpoint inhibitor therapy, thereby minimising, reducing or preventing metastasis in the subject having cancer.

In any aspect or embodiment, the invention further provides a method of minimising, reducing or preventing metastasis in a subject having cancer comprising:
identifying a subject having a primary tumor;
removing the primary tumor from the subject; and
administering to the subject a radiolabelled agent (such as a radiolabelled antigen binding protein) that binds to or specifically binds to CAIX, in combination with immune checkpoint inhibitor therapy,
   thereby minimising, reducing or preventing metastasis in the subject having cancer.

In another aspect, the present invention further provides a method of minimising, reducing or preventing growth of a tumor in at least one site distant from the site of another tumor (e.g. the primary tumor) in a subject comprising administering to the subject a radiolabelled agent (preferably a radiolabelled antigen binding protein) that binds to or specifically binds to CAIX, in combination with immune checkpoint inhibitor therapy, thereby minimising, reducing or preventing minimising, reducing or preventing growth of a tumor in at least one site distant from the site of the primary tumor in the subject.

In any aspect or embodiment of the invention, immune checkpoint inhibitor therapy may comprise administering one or more immune checkpoint inhibitors.

In any aspect or embodiment of the invention, the methods may further comprise a step of conjugating an antigen binding protein that binds to or specifically binds to CAIX with a radionuclide to provide a radiolabelled antigen binding protein that binds to or specifically binds to CAIX.

In another aspect, the present invention further provides use of an agent comprising, consisting or consisting essentially of a small molecule, peptide or antigen binding protein that binds to or specifically binds to CAIX, and/or a checkpoint inhibitor, in the manufacture of a medicament for treating, preventing or minimising progression of cancer in a subject. Preferably, the agent is conjugated to or capable of being conjugated to a radionuclide.

In another aspect, the present invention further provides use of an agent that binds to or specifically binds to CAIX (such as a small molecule, peptide or antigen binding protein, preferably an antigen binding protein) in the manufacture of a first medicament, and a checkpoint inhibitor in the manufacture of a second medicament, wherein the first and second medicaments are for:
treating, preventing or minimising progression of cancer in a subject,
minimising, reducing or preventing growth of a tumor in a subject,
minimising, reducing or preventing metastasis in a subject, or
increasing survival of a subject,
   preferably, wherein the agent is conjugated to a radionuclide.

Alternatively, the first and second medicaments are for any other method or use of the invention as described herein.

In another aspect, the present invention further provides an agent that binds to or specifically binds to CAIX (such as a small molecule, peptide or antigen binding protein, preferably an antigen binding protein), wherein the agent is conjugated to a radionuclide, or capable of being conjugated to a radionuclide, for use in combination with immune checkpoint inhibitor therapy, for treating, preventing, or preventing progression of cancer in a subject.

In another aspect, the present invention further provides use of an agent (such as a small molecule, peptide or antigen binding protein, preferably an antigen binding protein), that binds to or specifically binds to CAIX, wherein the agent is conjugated to a radionuclide, and an immune checkpoint inhibitor, for treating, preventing, or preventing progression of cancer in a subject.

In another aspect, the present invention further provides an agent (such as a small molecule, peptide or antigen binding protein, preferably an antigen binding protein) that binds to or specifically binds to CAIX, wherein the agent is conjugated to a radionuclide, for use in combination with immune checkpoint inhibitor therapy, in treating, preventing, or preventing progression of cancer in a subject. Alternatively, the agent that binds to or specifically binds to CAIX and the checkpoint inhibitor is for use in any other method or use of the invention as described herein including treating, preventing or minimising progression of cancer in a subject, minimising, reducing or preventing growth of a tumor in a subject, minimising, reducing or preventing metastasis in a subject, or increasing survival of a subject.

In another aspect, the present invention further provides the use of an agent (such as a small molecule, peptide or antigen binding protein, preferably an antigen binding protein) that binds to or specifically binds to CAIX, in the manufacture of a medicament for:
treating, preventing or minimising progression of cancer in a subject who has received or who is receiving a checkpoint inhibitor,
minimising, reducing or preventing growth of a tumor in a subject who has received or who is receiving a checkpoint inhibitor,
minimising, reducing or preventing metastasis in a subject who has received or who is receiving a checkpoint inhibitor, or
increasing survival of a subject who has received or who is receiving a checkpoint inhibitor,
preferably, wherein the agent is conjugated to or capable of being conjugated to a radionuclide.

In any aspect or embodiment of the invention, any medicament described herein is suitable for administration intraperitoneally, intratumorally, topically, orally, intravenously, to the respiratory tract, preferably by inhalation or intranasally, subcutaneously or intramuscularly. Typically, any medicament described herein is suitable for administration intravenously.

In another aspect, the present invention further provides an antigen binding protein that binds to or specifically binds to CAIX, for use in treating, preventing or minimising progression of cancer in a subject who has received or who is receiving a checkpoint inhibitor, preferably, wherein the antigen binding protein is conjugated to a radionuclide. Alternatively, the antigen binding protein that binds to or specifically binds to CAIX is for use in any other method or use of the invention as described herein. In an aspect of the invention, the antigen binding protein that binds to or specifically binds to CAIX preferably, wherein the antigen binding protein is conjugated to a radionuclide, is suitable for administration intraperitoneally, intratumorally, topically, orally, to the respiratory tract, preferably by inhalation or intranasally, intravenously, subcutaneously or intramuscularly. Preferably, the antigen binding protein that binds to or specifically binds to CAIX is suitable for administration intravenously.

In another aspect, the present invention further provides use of an antigen binding protein that binds to or specifically binds to CAIX, preferably, wherein the antigen binding protein is conjugated to a radionuclide, for treating, preventing or minimising progression of cancer in a subject who has received or who is receiving a checkpoint inhibitor. Alternatively, the use of an antigen binding protein that binds to or specifically binds to CAIX is in any other method or use of the invention as described herein.

In any of the uses described herein, the peptide or antigen binding protein that binds to or specifically binds to CAIX may be conjugated with a radionuclide through a linker or chelator. The radionuclide may be any of: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 and radium-224 (²²³Ra, ²²⁴Ra), samarium-153 (¹⁵³Sm), scandium-47 (⁴⁷Sc), strontium-90 (⁹⁰Sr), and yttrium-90 (⁹⁰Y). In some embodiments, the radionuclide is lutetium-177.

In any embodiment of the invention, the therapeutic effect of any radioconjugated agent (such as an antigen binding protein) that binds to or specifically binds to CAIX and checkpoint inhibitor as described herein, may be significant compared to the effect of the radioconjugated agent or the checkpoint inhibitor alone (ie compared to monotherapy with the radioconjugated agent that binds to or specifically binds to CAIX, or monotherapy with the checkpoint inhibitor). In an aspect, the effect may be additive or synergistic. Preferably, the effect is synergistic.

In another aspect, when the radioconjugated agent (preferably radiolabelled antigen binding protein) that binds to or specifically binds to CAIX and checkpoint inhibitor are administered to a subject, the radioconjugated agent improves the effectiveness of the checkpoint inhibitor. Optionally, the radiolabelled agent (preferably antigen binding protein) improves the effectiveness of the checkpoint inhibitor to the extent that the overall dose of checkpoint inhibitor that is administered to the subject, may be a lower dose than required to achieve the same effect, when the checkpoint inhibitor is administered alone (ie monotherapy). Preferably, the improvement is such that the dose of checkpoint inhibitor may be reduced by at least 10%, at least 25%, at least 35%, at least 50%, at least 75% or more, compared to the dose required for monotherapy with the checkpoint inhibitor. More preferably, the improvement is such that the dose of checkpoint inhibitor may be at least a quarter, at least a third, at least a half, or at least two thirds of the dose required for monotherapy with the checkpoint inhibitor

In another aspect, when the radioconjugated agent (preferably antigen binding protein) that binds to or specifically binds to CAIX, and checkpoint inhibitor described herein are administered to a subject, the checkpoint inhibitor can improve the effectiveness of the radioconjugated agent (preferably antigen binding protein). Optionally, the checkpoint inhibitor improves the effectiveness of the agent (preferably antigen binding protein) to the extent that the overall dose of agent that is administered to the subject, may be a lower dose than the dose required to achieve the same effect, when the agent is administered alone. Preferably, the improvement is such that the dose of radioconjugated agent (preferably antigen binding protein) that specifically binds to CAIX, may be reduced by at least 10%, at least 25%, at least 35%, at least 50%, at least 75% or more compared to the dose required for monotherapy with the radioconjugated agent (preferably antigen binding protein). More preferably, the improvement is such that the dose of radioconjugated agent (preferably antigen binding protein) may be at least a quarter, at least a third, at least a half, or at least two thirds of the dose required for monotherapy with the radioconjugated agent (antigen binding protein).

In certain embodiments, the effect of the radioconjugated agent (preferably antigen binding protein) that binds to or specifically binds to CAIX and checkpoint inhibitor described herein on survival of the subject may be significantly greater than the effect of the agent and checkpoint inhibitor when administered alone. In a further embodiment, the effect of any radioconjugated agent that binds to or specifically binds to CAIX, and checkpoint inhibitor described herein on tumor growth in the subject may be significantly greater than the effect of the agent and checkpoint inhibitor when administered alone.

In any aspect of the invention, the radioconjugated agent (preferably antigen binding protein) that binds to or specifically binds to CAIX, and/or checkpoint inhibitor are administered once. In another embodiment, the agent (preferably antigen binding protein) and/or checkpoint inhibitor are administered two, three, four or more times to the subject.

In any aspect of the invention, the radioconjugated agent (antigen binding protein) that binds to or specifically binds to CAIX, and/or checkpoint inhibitor may be administered in the same composition or in separate compositions. In another aspect, the agent (antigen binding protein) and/or checkpoint inhibitor may therefore be administered together or sequentially. Alternatively, administration may be staggered. The agent (antigen binding protein) and/or checkpoint inhibitor may also be administered at the same frequency or at different frequencies.

Optionally, the radioconjugated agent (antigen binding protein) that binds to or specifically binds to CAIX, is administered to the subject prior to the administration of the checkpoint inhibitor. In some examples, the radioconjugated agent that binds to or specifically binds to CAIX is administered to the subject at least 24 hours, at least 2 days, at least 3 days, at least 4 days, at least 5 days, at least 6 days or at least 7 days prior to the administration of the checkpoint inhibitor. Preferably, the radioconjugated agent (antigen binding protein) that binds to or specifically binds to CAIX is administered at least 1 day before the administration of the checkpoint inhibitor.

In any aspect of the invention, the antigen binding protein that binds to or specifically binds to CAIX and the checkpoint inhibitor may be administered by any known administration routes in the art including intraperitoneally, intratumorally, topically, orally, to the respiratory tract via inhalation or intranasally, intravenously, subcutaneously or intramuscularly. Preferably, antigen binding protein that binds to or specifically binds to CAIX, conjugated with a radionuclide, and/or checkpoint inhibitor are administered intravenously.

In any aspect of the invention, the amount of antigen binding protein that binds to or specifically binds to CAIX, conjugated with a radionuclide administered may be in the range of about 250 nmoles/kg body weight/dose to 0.005 nmoles/kg body weight/dose.

In any aspect of the invention, the amount of antigen binding protein that binds to or specifically binds to CAIX, conjugated with a radionuclide administered may be in the range of about 0.1, 0.2, 0.3, 0.4, 0.5, 0.6, 0.7, 0.8, 0.9, 1.0, 5, 10, 15, 20, 25, 30, 35, 40, 45, 50, 55, 60, 65, 70, 75, 80, 85, 90, 95 or 100 μg/kg or more.

In any aspect of the invention, the amount of checkpoint inhibitor administered may be in the range of from about 0.001 to about 100 mg/kg, about 0.001 to about 75 mg/kg, about 0.001 to about 50 mg/kg, about 0.001 to about 25 mg/kg, about 0.001 to about 20 mg/kg, about 0.005 to about 20 mg/kg, about 0.01 to about 20 mg/kg, about 0.01 to about 20 mg/kg, about 0.1 to about 10 mg/kg, about 0.1 to about 5 mg/kg, about 1 to about 5 mg/kg, about 2 to about 5 mg/g, about 7.5 to about 12.5 mg/kg, or about 0.1 to about 30 mg/kg of the subject's body weight. For example, dosages can be about 0.01, about 0.1, about 0.3, about 1, about 2, about 3, about 5 or about 10 mg/kg body weight, or, about 0.3, about 1, about 2, about 3, about 5 mg/kg, or about 10 mg/kg body weight.

In any aspect of the invention, the cancer may be any cancer associated with or caused by expression of CAIX. In some embodiments, the cancer is selected from the group consisting of breast cancer, colorectal cancer, adenocarcinomas, mesothelioma, bladder cancer, prostate cancer, germ cell cancer, hepatoma/cholongio carcinoma, neuroendocrine cancer, pituitary neoplasm, small round cell tumor, squamous cell cancer, melanoma, atypical fibroxanthoma, seminomas, nonseminomas, stromal leydig cell tumors, Sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, fibrosarcoma, vaginal tumors, blood cancers or Wilm's tumor. In a preferred embodiment, the cancer is melanoma, breast cancer or colon cancer. In some embodiments, the cancer is a colorectal cancer or a renal cancer, such as renal cortical adenocarcinoma or clear cell renal cell carcinoma (ccRCC).

In any aspect of the invention, the checkpoint inhibitor may be a PD-1, PD-L1 or a CTLA-4 checkpoint inhibitor. In an aspect, the checkpoint inhibitor is an antibody. In some embodiments, the checkpoint inhibitor is an inhibitor of PD-1, PD-L1 or CTLA-4 in the form of an antibody. In some embodiments, the checkpoint inhibitor is an inhibitor of PD-1. In some embodiments, the checkpoint inhibitor is an inhibitor of CTLA-4. In some embodiments, immune checkpoint inhibitor therapy involves administering inhibitors of PD-1 and CTLA-4. Examples of such inhibitors are further described herein.

Antigen Binding Protein that Binds to or Specifically Binds to CAIX

The invention involves administering an antigen binding protein that binds to or specifically binds to CAIX, wherein the antigen binding protein is conjugated to a radionuclide.

In any embodiment, the radiolabelled antigen binding protein is an antibody, or antigen binding fragment thereof, for binding to CAIX. Any suitable antibody or antigen binding fragment thereof for binding to CAIX may be used. In some embodiments, the radiolabelled antigen binding protein that binds to or specifically binds to CAIX is radiolabelled girentuximab or a radiolabelled functional variant or fragment thereof that retains the ability to bind to CAIX. In some embodiments, the radiolabelled antigen binding protein that binds to or specifically binds to CAIX is radiolabelled G250. In some embodiments, the radiolabelled antigen binding protein that binds to or specifically binds to CAIX is a radiolabelled chimeric antibody or antigen binding fragment thereof. In some embodiments, the radiolabelled antigen binding protein that binds to or specifically binds to CAIX is a radiolabelled humanised antibody or radiolabelled antigen binding fragment thereof. Optionally, the radiolabelled antigen binding protein is radiolabelled humanised G250 (hG250).

In any embodiment, the antigen binding protein that binds to or specifically binds to CAIX is as described in any of WO 2002/062972 A2 (US 2004/0219633 A1), WO 2004/002526 A1 (U.S. Pat. No. 7,632,496 B2), WO 2006/002889 A2 (U.S. Pat. No. 7,691,375 B2), WO 2009/056342 A1 (US 2014/0017252 A1), WO 2011/032973 A1 (US 2012/0207672 A1), WO 2014/128258 A1 (U.S. Pat. No. 10,620,208 B2) or U.S. Pat. No. 11,629,199. One antibody for binding to CAIX that is described in these publications is referred to herein as TLX250, which is further described herein in the Examples. In any embodiment, the antibody or antigen binding fragment for binding to CAIX is as described in WO 2021/000017 A1. The entire contents of each of these publications is incorporated herein by reference.

In any embodiment, the antigen binding protein comprises:
(a) the three CDRs of an antigen binding domain having a heavy chain variable domain (VH) as set forth in SEQ ID NOs: 4, 20, 36, 52 or 68; and/or
(b) the three CDRs of an antigen binding domain having a light chain variable domain (VL) comprising the amino acid sequence as set forth in SEQ ID NOs: 84, 100, 116, 132, 148 or 164.

The skilled person will be familiar with various methods for determining the CDRs of any given heavy or light variable chain, including using methods conventionally used in the art and described elsewhere herein. Moreover the skilled person will appreciate that the definition of the CDR boundaries for any given variable chain will depend on the particular naming convention used (such as the IMGT, Chothia or Kabat numbering systems).

In any embodiment, the antigen binding protein comprises:
(a) a heavy chain variable domain (VH) comprising three complementarity determining regions (CDRs) of the amino acid sequence as set forth in SEQ ID NOs: 4, 20, 36, 52 or 68; and/or
(b) a light chain variable domain (VL) comprising three complementarity determining regions (CDRs) of the amino acid sequence as set forth in SEQ ID NOs: 84, 100, 116, 132, 148 or 164.

In preferred embodiments, the antigen binding protein comprises the amino acid sequences of a VH and a VL as set forth in:
SEQ ID NO: 4 and SEQ ID NO: 84
SEQ ID NO: 36 and SEQ ID NO: 132
SEQ ID NO: 52 and SEQ ID NO: 132
SEQ ID NO: 52 and SEQ ID NO: 148;
   or a variant thereof, wherein the VH and VL comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions or additions, compared to the amino acid sequences as set forth above; wherein the amino acid substitutions, deletions or additions are not in the CDRs and the antigen binding protein retains the ability to bind to CAIX.

In any embodiment, the antigen binding protein comprises an antigen binding domain that binds specifically to carbonic anhydrase IX (CAIX) and comprises:

FR1-CDR1-FR2-CDR2-FR3-CDR3-FR4-linker-FR1a-CDR1a-FR2a CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The CDR1, CDR2 and CDR3 may be sequences from the VH, CDR1a, CDR2a and CDR3a may be sequences from VL, or the CDR1, CDR2 and CDR3 may be sequences from the VL, CDR1a, CDR2a and CDR3a may be sequences from VH.

Reference herein to a protein or antibody that “binds to” carbonic anhydrase IX (CAIX) provides literal support for a protein or antibody that “binds specifically to” or “specifically binds to” CAIX.

In any embodiment, the antigen binding protein comprises:
(i) a VH comprising a complementarity determining region (CDR) 1 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 1, a CDR2 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set in SEQ ID NO: 2, and a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 3;
(ii) a VH comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in any of SEQ ID NO: 4, 20, 36, 52 or 68;
(iii) a VL comprising a CDR1 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 81, a CDR2 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 82 and a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 83;
(iv) a VL comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 84, 100, 116, 132, 148 or 164;
(v) a VH comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3;
(vi) a VH comprising a sequence set forth in any of SEQ ID NO: 4, 20, 36, 52 or 68;
(vii) a VL comprising a CDR1 comprising a sequence set forth in SEQ ID NO: 81, a CDR2 comprising a sequence set forth in SEQ ID NO: 82, and a CDR3 comprising a sequence set forth in SEQ ID NO: 83;
(viii) a VL comprising a sequence set forth in any of SEQ ID NO: 84, 100, 116, 132, 148 or 164;
(ix) a VH comprising a CDR1 comprising a sequence set forth in SEQ ID NO: 1, a CDR2 comprising a sequence set forth between in SEQ ID NO: 2, and a CDR3 comprising a sequence set forth in SEQ ID NO: 3; and a VL comprising a CDR1 comprising a sequence set SEQ ID NO: 81, a CDR2 comprising a sequence set forth in SEQ ID NO: 82, and a CDR3 comprising a sequence set forth in SEQ ID NO: 83; or
(x) a VH comprising a sequence set forth in any of SEQ ID NO: 4, 20, 36, 52 or 68 and a VL comprising a sequence set forth in any of SEQ ID NO: 84, 100, 116, 132, 148 or 164.

In a further embodiment, the antigen binding protein comprises:
(i) a VH comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID Nos: 9, 25, 41, 57 or 73; a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID Nos: 10, 26, 42, 58 or 74; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID Nos: 11, 27, 43, 59 or 75; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID Nos: 12, 28, 44, 60 or 76; and
(ii) a VL comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID Nos: 89, 105, 121, 137, 153 or 169; a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID Nos: 90, 106, 122, 138, 154 or 170; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID Nos: 91, 107, 123, 139, 155 or 171; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID Nos: 92, 108, 124, 140, 156 or 172.

In a further embodiment, the antigen binding protein comprises:
(i) a VH comprising a framework region (FR) 1 comprising or consisting of a sequence as set forth in SEQ ID Nos: 9, 25, 41, 57 or 73; a FR2 comprising or consisting of a sequence as set forth in SEQ ID Nos: 10, 26, 42, 58 or 74; a FR3 comprising or consisting of a sequence as set forth in SEQ ID Nos: 11, 27, 43, 59 or 75; a FR4 comprising or consisting of a sequence as set forth in SEQ ID Nos: 12, 28, 44, 60 or 76, and
(ii) a VL comprising a framework region (FR) 1 comprising or consisting of a sequence as set forth in SEQ ID Nos: 89, 105, 121, 137, 153 or 169; a FR2 comprising or consisting of a sequence as set forth in SEQ ID Nos: 90, 106, 122, 138, 154 or 170; a FR3 comprising or consisting of a sequence as set forth in SEQ ID Nos: 91, 107, 123, 139, 155 or 171; a FR4 comprising or consisting of a sequence as set forth in SEQ ID Nos: 92, 108, 124, 140, 156 or 172.

In any embodiment, the antibody that specifically binds to CAIX comprises an antigen binding protein that consists essentially of or consists of an amino acids sequence of (in order of N to C terminus or C to N terminus) any one of SEQ ID NO: 4, 20, 36, 52 or 68 and/or any one of SEQ ID NO: 84, 100, 116, 132, 148, 164.

In any embodiment, the antigen binding protein comprises:
(a) a heavy chain variable domain (VH) comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NOs: 4, 20, 36, 52 or 68; and/or
(b) a light chain variable domain (VL) comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NOs: 84, 100, 116, 132, 148 or 164. In some embodiments, the antigen binding protein is a modified IgG antibody, comprising a heavy chain constant region having one or more amino acid substitutions compared to a wild-type antibody of the class IgG, wherein the one or more amino acid substitutions reduce the affinity of the antibody for the neonatal Fc receptor (FcRn), thereby reducing the serum half-life of the modified antibody compared to a wild-type antibody of class IgG.

In one embodiment, the one or more amino acid substitutions are selected from substitutions in the heavy chain constant region 2 (CH2) of the IgG molecule, reducing the affinity of the IgG molecule for FcRn. Alternatively, the one or more amino acid substitutions may be in the heavy chain constant region 3 (CH3) of the IgG molecule, thereby reducing the affinity of the IgG molecule for FcRn. Still further, the amino acid substitutions may include at least one substitution in the CH2 region, and at least one substitution in the CH3 region of the IgG molecule, whereby the substitutions reduce the affinity of the IgG for FcRn.

In certain preferred embodiments, the one or more amino acid substitutions may be at one or more of residues His310, His433, His435, His436, or Ile253 of IgG. Preferably, the amino acid substitutions comprise a substitution in the heavy chain constant region at positions His310 or at His435. More preferably, the amino acid substitutions that reduce the affinity of the antibody for FcRn are at both His310 and His435.

In certain embodiments, the modified antibody retains the ability to bind to one or more Fc-gamma receptors and accordingly, in certain embodiments the modified antibody retains the ability to stimulate effector responses (including ADCC).

In alternative embodiments, the one or more amino acid modifications which reduce the affinity for the FcRn receptor also reduce the affinity for the Fc gamma receptors. The modified antibody may further comprise one or more amino acid substitutions compared a wild-type antibody of the class IgG, wherein the amino acid substitutions further reduce the affinity of the antibody for one or more Fc gamma receptors.

In a further embodiment, the modified antibody further comprises one or more amino acid substitutions compared a wild-type antibody of the class IgG, wherein the amino acid substitutions increase the stability of the CH1-CH2 hinge region in the modified antibody compared to a wild-type antibody of the class IgG.

In any embodiment, the antibody for binding to CAIX is conjugated to a therapeutic agent. The therapeutic agent may be conjugated to the antibody directly or indirectly, e.g. by halogenation of amino acid residues. Preferably, the therapeutic agent is indirectly conjugated to the antibody by way of a linker or chelator moiety. In one example, the antibody is conjugated to a chelating moiety, selected from the group consisting of: TMT (6,6″-bis[N,N″,N″′-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N″-tetraacetic acid), TCMC, DO3A, CB-DO2A, NOTA, Diamsar, DTPA, CHX-A″-DTPA, TETE, Te2A, HBED, DFO, DFOsq, DFO-NCS, HOPO or a chelator as described in WO 2022/133537 (incorporated herein by reference) or other chelating agent as described herein.

In another example, the antibody is conjugated to a bifunctional linker, for example, bromoacetyl, thiols, succinimide ester, TFP ester, a maleimide, or using any amine or thiol-modifying chemistry known in the art.

Preferably, the therapeutic agent is a radioisotope. Examples of suitable isotopes include: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 and radium-224 (²²³Ra, ²²⁴Ra), samarium-153 (¹⁵³Sm), scandium-47 (⁴⁷Sc), strontium-90 (⁹⁰Sr), and yttrium-90 (⁹⁰Y).

In any embodiment, the heavy chain constant region of the antibody comprises amino acid substitutions at both His310 and His435. The antibody may also comprise amino acid substitutions at residues equivalent to Ser228 and Leu235 of the constant heavy chain region.

Preferably, the antibody comprises a heavy chain constant region comprising the sequence as set forth in any one of SEQ ID NOs:177 to 180, preferably as set forth in SEQ ID NO: 178.

In a still further embodiment, the antibody preferably comprises a heavy chain comprising the sequence set forth in any one of SEQ ID NOs: 182 to 185, preferably as set forth in SEQ ID NO: 183.

In any embodiment, the antibody comprises a light chain constant region comprising the amino acid sequence as set forth in SEQ ID NO: 181. Preferably, the antibody comprises a light chain comprising the amino acid sequence as set forth in SEQ ID NO: 186.

In any embodiment, the antibody comprises the sequence set forth in SEQ ID NO: 183 and the sequence set forth in SEQ ID NO: 186.

The present invention also provides a use of molecule comprising an immunoglobulin moiety and a non-protein agent conjugated thereto,
wherein, the immunoglobulin moiety specifically binds to CAIX and comprises: an antigen binding protein that consists essentially of or consists of an amino acid sequence of (in order of N to C terminus or C to N terminus) any one of SEQ ID NOs: 4, 20, 36, 52 or 68 and any one of SEQ ID Nos 84, 100, 116, 132, 148, 164,
optionally, wherein the immunoglobulin moiety has reduced or abolished affinity for the FcRn receptor compared to a wild-type immunoglobulin; and
wherein the non-protein agent comprises a therapeutic moiety such as a radioactive element.

Preferably, the immunoglobulin moiety has amino acid substitutions at residues equivalent to His310 and/or His435 in the constant heavy chain region. The immunoglobulin moiety may also comprise amino acid substitutions at residues equivalent to Ser228 and Leu235 of the constant heavy chain region. Preferably, the non-protein agent comprises a radioactive element.

The present invention also provides a use of molecule comprising an immunoglobulin moiety and a non-protein agent conjugated thereto, optionally wherein, the immunoglobulin moiety has reduced or abolished affinity for the FcRn receptor compared to a wild-type immunoglobulin
wherein the immunoglobulin moiety specifically binds to CAIX and comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 1, 17, 33, 49 or 65, a CDR2 comprising a sequence at least about 80%, at least 85%, at least 90%, at least 92%, at least 95%, at least 97%, at least 99% identical to a sequence set in SEQ ID NO: 2, 18, 34, 50 or 66, and a CDR3 comprising a sequence at least about 80%, at least 85%, at least 90%, at least 92%, at least 95%, at least 97%, at least 99% identical to a sequence set forth in SEQ ID NO: 3, 19, 35, 51 or 67;
(ii) a VH comprising a sequence at least about 95% or 96% or 97% or 98% or 99% identical to a sequence set forth in SEQ ID NO: 4, 20, 36, 52 or 68;
(iii) a VL comprising a CDR1 comprising a sequence at least about 80%, at least 85%, at least 90%, at least 92%, at least 95%, at least 97%, at least 99% identical to a sequence set forth in SEQ ID NO: 81, 97, 113, 129, 145 or 161, a CDR2 comprising a sequence at least about 80%, at least 85%, at least 90%, at least 92%, at least 95%, at least 97%, at least 99% identical to a sequence set forth in SEQ ID NO: 82, 98, 114, 130, 146 or 162 and a CDR3 comprising a sequence at least about 80%, at least 85%, at least 90%, at least 92%, at least 95%, at least 97%, at least 99% identical to a sequence set forth in SEQ ID NO: 83, 99, 115, 131, 147 or 163;
(iv) a VL comprising a sequence at least about 95% identical to a sequence set forth in SEQ ID NO: 84, 100, 116, 132, 148 or 164;
(v) a VH comprising a CDR1 comprising a sequence set forth in SEQ ID NO: 1, 17, 33, 49 or 65, a CDR2 comprising a sequence set forth between in SEQ ID NO: 2, 18, 34, 50 or 66 and a CDR3 comprising a sequence set forth in SEQ ID NO: 3, 19, 35, 51 or 67;
(vi) a VH comprising a sequence set forth in SEQ ID NO: 4, 20, 36, 52 or 68;
(vii) a VL comprising a CDR1 comprising a sequence set SEQ ID NO: 81, 97, 113, 129, 145 or 161, a CDR2 comprising a sequence set forth in SEQ ID NO: 82, 98, 114, 130, 146 or 162 and a CDR3 comprising a sequence set forth in SEQ ID NO: 83, 99, 115, 131, 147 or 163;
(viii) a VL comprising a sequence set forth in SEQ ID NO: 84, 100, 116, 132, 148 or 164;
(ix) a VH comprising a CDR1 comprising a sequence set forth in SEQ ID NO: 1, 17, 33, 49 or 65, a CDR2 comprising a sequence set forth between in SEQ ID NO: 2, 18, 34, 50 or 66 and a CDR3 comprising a sequence set forth in SEQ ID NO: 3, 19, 35, 51 or 67; and a VL comprising a CDR1 comprising a sequence set SEQ ID NO: 81, 97, 113, 129, 145 or 161, a CDR2 comprising a sequence set forth in SEQ ID NO: 82, 98, 114, 130, 146 or 162 and a CDR3 comprising a sequence set forth in SEQ ID NO: 83, 99, 115, 131, 147 or 163; or
(x) a VH comprising a sequence set forth in SEQ ID NO: 4, 20, 36, 52 or 68 and a VL comprising a sequence set forth in SEQ ID NO: 84, 100, 116, 132, 148 or 164.

Preferably, the non-protein agent comprises a radioactive element. Preferably, the immunoglobulin moiety has amino acid substitutions at residues equivalent to His310 and/or His435 in the constant heavy chain region. The immunoglobulin moiety may also comprise amino acid substitutions at residues equivalent to Ser228 and Leu235 of the constant heavy chain region. Preferably, the non-protein agent comprises a radioactive element.

In any embodiment, the immunoglobulin comprises a heavy chain constant region comprising the sequence as set forth in any one of SEQ ID NOs:177 to 180, preferably as set forth in SEQ ID NO: 178.

In a still further embodiment, the immunoglobulin comprises a heavy chain comprising the sequence set forth in any one of SEQ ID NOs:182 to 185, preferably as set forth in SEQ ID NO: 183.

In any embodiment, the immunoglobulin comprises a light chain constant region comprising a sequence as set forth in SEQ ID NO: 181. In an embodiment, the light chain comprises the sequence of SEQ ID NO: 186.

In a particular preferred embodiment, the immunoglobulin comprises the amino acid sequences a set forth in SEQ ID NOs:183 and 186.

In certain embodiments, the antibody is an antibody as described herein, including an antibody having any of the complementarity determining regions, framework regions, variable light or variable heavy regions as described in Table 1 below. Preferably, the modified antibody also comprises amino acid substitutions at residues equivalent to Ser228 and Leu235 of the constant heavy chain region. Preferably, the radioactive element is conjugated to the modified antibody using a chelating agent, for example DOTA.

In any embodiment, the modified antibody for a use as described herein, has reduced toxicity compared to an antibody that is not modified. The reduced toxicity includes reducing a number of toxic effects which would otherwise result from longer-term residence of radioisotope in the circulation (including haematological toxicity, absorption into bone and bone marrow irradiation).

In any embodiment, the toxicity of a radio-labelled antibody or radioimmunoconjugate herein described is assessed by determining the tumor:blood ratio of the antibody or immunoconjugate following administration to an individual.

In any embodiment of the invention, the tumor:blood ratio of the modified antibodies of the present invention is at least 2 times greater, at least 3 times greater, at least 4 times greater, at least 6 times greater, at least 8 times greater or at least 10 times greater, or more, than for unmodified antibodies that do not have the modifications to the heavy chain constant region as herein described, when the ratio is determined at least 8 hours following administration of the antibody. Alternatively, the ratio is determined at least 24, 48, 72 or 120 hours following administration of the antibody to an individual. In certain embodiments, the tumor:blood ratio of the modified antibodies of the present invention is at least 50 times greater, at least 100 times greater, at least 200 times greater or at least 300 times greater than for unmodified antibodies that do not have the modifications to the heavy chain constant region as herein described, when the ratio is determined at least 120 hours following administration of the antibody.

In any embodiment of the invention, the modified antibodies herein described having reduced or altered serum half-life compared to unmodified antibodies, have a serum clearance rate that is at least two times faster, at least three times faster or more, than the unmodified antibodies.

In certain embodiments of the invention, the CAIX binding antibodies described herein are suitable for use in a theranostic pair, wherein the theranostic pair comprises 1) the antibody coupled to an imaging agent and 2) the antibody coupled to an agent for therapy. For example, the antibody may firstly be used as a diagnostic when coupled to a radioisotope suitable for use in radioimaging, Secondly, the antibody may be used as a therapeutic when coupled to a radioisotope or cytotoxic agent suitable for use in therapy.

In further embodiments, the methods of the invention may also include a step of diagnosing, monitoring or prognosing a disease, disorder or infection in a subject comprising:
(a) administering to a subject an antibody as herein described, said antibody specifically binding to an antigen associated with a disease, disorder or infection;
(b) allowing the antibody to concentrate at sites in said subject where said antigen is found; and
(c) detecting said antibody;
whereby detection of said antibody above a background or standard level indicates that the subject has said disease disorder or infection.

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO: 9, 25, 41, 57 or 76, a FR2 comprising a sequence at least about 80%, at least 85%, at least 90%, at least 92%, at least 95%, at least 97%, at least 99% identical to a sequence set in SEQ ID NO: 10, 26, 42, 58 or 74, a FR3 comprising a sequence at least about 80%, at least 85%, at least 90%, at least 92%, at least 95%, at least 97%, at least 99% identical to a sequence set forth in SEQ ID NO: 11, 27, 43, 59 or 75, and a FR4 comprising a sequence at least about 80%, at least 85%, at least 90%, at least 92%, at least 95%, at least 97%, at least 99% identical to a sequence set forth in SEQ ID NO: 12, 28, 44, 60 or 76;
(ii) a VL comprising a FR1 comprising a sequence at least about 80%, at least 85%, at least 90%, at least 92%, at least 95%, at least 97%, at least 99% identical to a sequence set forth in SEQ ID NO: 89, 105, 121, 137, 153, or 169, a FR2 comprising a sequence at least about 80%, at least 85%, at least 90%, at least 92%, at least 95%, at least 97%, at least 99% identical to a sequence set forth in SEQ ID NO: 90, 106, 122, 138, 154, or 170, a FR3 comprising a sequence at least about 80%, at least 85%, at least 90%, at least 92%, at least 95%, at least 97%, at least 99% identical to a sequence set forth in SEQ ID NO: 91, 107, 123, 139, 155, or 171, and a FR4 comprising a sequence at least about 80%, at least 85%, at least 90%, at least 92%, at least 95%, at least 97%, at least 99% identical to a sequence set forth in SEQ ID NO: 92, 108, 124, 140, 156, or 172;
(iii) a VH comprising a FR1 comprising a sequence set forth in SEQ ID NO: 9, 25, 41, 57 or 76, a FR2 comprising a sequence set forth between in SEQ ID NO: 10, 26, 42, 58 or 74, a FR3 comprising a sequence set forth in SEQ ID NO: 11, 27, 43, 59 or 75, and a FR4 comprising a sequence set forth in SEQ ID NO: 12, 28, 44, 60 or 76;
(iv) a VL comprising a FR1 comprising a sequence set forth in SEQ ID NO: 89, 105, 121, 137, 153, or 169, a FR2 comprising a sequence set forth between in SEQ ID NO: 90, 106, 122, 138, 154, or 170, a FR3 comprising a sequence set forth in SEQ ID NO: 91, 107, 123, 139, 155, or 171, and a FR4 comprising a sequence set forth in SEQ ID NO: 92, 108, 124, 140, 156, or 172; or
(v) a VH comprising a FR1 comprising a sequence set forth in SEQ ID NO: 9, 25, 41, 57 or 76, a FR2 comprising a sequence set forth between in SEQ ID NO: 10, 26, 42, 58 or 74, a FR3 comprising a sequence set forth in SEQ ID NO: 11, 27, 43, 59 or 75, and a FR4 comprising a sequence set forth in SEQ ID NO: 12, 28, 44, 60 or 76; and a VL comprising a FR1 comprising a sequence set forth in SEQ ID NO: 89, 105, 121, 137, 153, or 169, a FR2 comprising a sequence set forth between in SEQ ID NO: 90, 106, 122, 138, 154, or 170, a FR3 comprising a sequence set forth in SEQ ID NO: 91, 107, 123, 139, 155, or 171, and a FR4 comprising a sequence set forth in SEQ ID NO: 92, 108, 124, 140, 156, or 172.

In one embodiment, the VH comprises a sequence at least about 95% or 96% or 97% or 98% or 99% identical to, or comprises a sequence set forth in SEQ ID NO: 36 or 52 and the VL comprises a sequence at least about 95% or 96% or 97% or 98% or 99% identical to, or comprises a sequence set forth in SEQ ID NO: 116, 132 or 148. Preferably, the VH comprises a sequence at least about 95% or 96% or 97% or 98% or 99% identical to, or comprises a sequence set forth in SEQ ID NO: 36 or 52 and the VL comprises a sequence at least about 95% or 96% or 97% or 98% or 99% identical to, or comprises a sequence set forth in SEQ ID NO: 132 or 148. More preferably, the VH comprises a sequence at least about 95% or 96% or 97% or 98% or 99% identical to, or comprises a sequence set forth in SEQ ID NO: 36 and the VL comprises a sequence at least about 95% or 96% or 97% or 98% or 99% identical to, or comprises a sequence set forth in SEQ ID NO: 148. Alternatively, the VH comprises a sequence at least about 95% or 96% or 97% or 98% or 99% identical to, or comprises a sequence set forth in SEQ ID NO: 52 and the VL comprises a sequence at least about 95% or 96% or 97% or 98% or 99% identical to, or comprises a sequence set forth in SEQ ID NO: 132 or 148, preferably the sequence set forth in SEQ ID NO: 148.

In any embodiment therein, an antigen binding protein can also be referred to as antigen binding domains of antibodies.

Preferably, an antigen binding protein as described herein is an antibody or antigen binding fragment thereof. Typically, the antigen binding protein is an antibody or antigen binding fragment thereof, for example, a monoclonal antibody.

As described herein, the antigen binding protein may be in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3; or
(iv) one of (i) or (ii) linked to a protein that binds to an immune effector cell.

Further, as described herein, the antigen binding protein may be in the form of:
(i) a diabody;
(ii) a triabody;
(iii) a tetrabody;
(iv) a Fab;
(v) a F(ab′)2;
(vi) a Fv;
(vii) one of (i) to (vi) linked to a constant region of an antibody, Fc or a heavy chain constant domain (CH) 2 and/or CH3; or
(viii) one of (i) to (vi) linked to a protein that binds to an immune effector cell.

In any aspect or embodiment, the antibody is a naked antibody. Specifically, the antibody is in a non-conjugated form and is not adapted to form a conjugate.

In any embodiment, the antigen binding protein for use according to the invention may be a fusion protein comprising an antigen binding protein, immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody as described herein.

In any embodiment, the antigen binding protein for use according to the invention may be a conjugate in the form of an antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody or fusion protein as described herein conjugated to a label or a cytotoxic agent In a preferred embodiment, the antigen binding protein is an IgG immunoglobulin comprising one or more amino acid substitutions in the antibody constant domain, CH2-CH3 region, which modify the binding of the antibody to the neonatal Fc receptor (FcRn) relative to a wild-type antibody Fc region. The one or more amino acid modifications change the affinity of the antibody constant domain, Fc region, or FcRn binding fragment thereof, for the FcRn and thereby alter the serum half-life of the antigen binding protein.

Preferably the substitution alters the binding affinity for FcRn and/or the serum half-life of said modified antibody relative to the unmodified wild-type antibody. The present invention further provides for a modified antibody having a reduced binding affinity for FcRn and/or a reduced serum half-life as compared with the unmodified antibody, wherein any one or more amino acid residues at positions Ile253 or His310 from the CH2 domain and/or residue His435 from the CH3 domain, is substituted with another amino acid which is different from that present in an unmodified antibody or to an unmodified IgG.

In one example, the one or more amino acid modifications is selected from an amino acid substitution at residue equivalent to H310 and H435. In a further example, the antibody comprises amino acid substitutions at both His310 and His435 residues.

In a further embodiment, the antibody may comprise an amino acid substitution at residue Lys322. Preferably the substitution is K322A. In particularly preferred embodiments, the antibody comprises substitutions K322A, H310A and H435Q.

The amino acid substitutions may include substitution from a histidine residue to: alanine, glutamine, glutamic acid or aspartic acid. Preferably, the amino acid substitution at His310 is to alanine. Preferably the amino acid substitution at His435 is to glutamine. Preferably, the amino acid substitution at Ile253 is alanine.

In a further embodiment, the antigen binding protein is an antibody or antigen binding fragment thereof that comprises one or more amino acid substitutions which modify the binding of the antibody to activating Fc gamma receptors. The one or more amino acid modifications change the affinity of the antibody constant domain, Fc region, or Fc gamma receptor binding fragment, for any one or more Fc gamma receptors. Preferably, the amino acid modification is at a residue equivalent to Leu235. More preferably, the amino acid modification is from Leu235 to glutamic acid.

In one embodiment, the amino acid modification is a hinge stabilising mutation at Ser228. Preferably the amino acid modification at Ser228 is to proline.

In one embodiment of the invention, the antibody comprises mutations at Ser228, Leu235, His310 and His435. Preferably, the amino acid modifications are Ser228Pro, Leu235Glu, His310Ala and His435Gln.

The amino acid modifications are preferably made in an antibody having an IgG1 isotype, or on an IgG4 isotype.

In a preferred embodiment, the antibody comprises a heavy chain constant region as set forth in any one of SEQ ID NOs: 177 to 180.

An antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein may be obtained by expressing a nucleic acid encoding the same.

In one example, such a nucleic acid is included in an expression construct in which the nucleic acid is operably linked to a promoter. Such an expression construct can be in a vector, e.g., a plasmid. In examples directed to single polypeptide chain antigen binding protein, the expression construct may comprise a promoter linked to a nucleic acid encoding that polypeptide chain. In examples directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates use of separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

An antigen binding protein or antibody or antigen binding fragment thereof as described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

In one example a protein or antibody as described herein or a composition of a protein or antibody as described herein, comprises a heavy chain constant region, comprising a stabilized heavy chain constant region, comprising a mixture of sequences fully or partially with or without the C-terminal lysine residue.

In one example, an antibody for use according to the invention comprises a VH disclosed herein linked or fused to an IgG4 constant region or stabilized IgG4 constant region (e.g., as discussed above) and the VL is linked to or fused to a kappa light chain constant region.

The functional characteristics of an antigen binding protein for use according to the invention will be taken to apply mutatis mutandis to an antibody of the invention.

An antigen binding protein as described herein may be purified, substantially purified, isolated and/or recombinant.

The present invention also provides a method for treating or preventing cancer in a subject, the method comprising administering an antigen binding protein as described herein to the subject. In this regard, an antigen binding protein can be used to prevent a relapse of a condition, and this is considered preventing the condition.

Other exemplary cancers include renal cancer. It will be understood that the antibodies of the invention having affinity for CAIX will be useful for this purpose.

The present invention also includes steps for an in vivo method of diagnosing, monitoring or prognosing a disease, disorder or infection in a subject prior to or in conjunction with administering a treatment as described herein.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Plot of area under tumor growth curves corrected for lifetime in Renca-hCAIX tumour-bearing mice following treatment described in Example 3 for the following treatment groups: (1) control, administration of targeted radionuclide therapy (TRT)-¹⁷⁷Lu-hG250 at (2) 12MBq, (3) 18MBq and (4) 24MBq, (5) administration of immune checkpoint inhibitor (ICI) anti-PD-1 Ab (200 μg) and (6) the combination of anti-PD-1 Ab (200 μg) with 18MBq ¹⁷⁷Lu-hG250. Analysis: Kruskal-Wallis test with Dunn's multiple comparison test *p<0.05, ***p<0.001. Pilot groups were excluded from analysis. N=10 mice per group for control and TRT monotherapy groups, and N=7 for ICI and combination groups.

FIG. 2: Plots of change of body weight of Renca-hCAIX tumour-bearing mice in each treatment group as described in Example 2. N=10 mice per group for control and TRT monotherapy groups, and N=7 for ICI and combination groups.

FIG. 3: Chart showing the reason for euthanisation of each Renca-hCAIX tumour-bearing mouse by treatment group as described in Example 2. N=10 mice per group to start for control and TRT monotherapy groups, and N=7 mice to start for ICI and combination groups.

FIG. 4: (A). Survival curves for Renca-hCAIX tumour-bearing mice by treatment group as described in Example 2. (B). Number of animals with complete tumour regression, and of those animals with complete tumour regressions, the number which rejected a subsequent implantation with either Renca cells or Renca-hCAIX cells. N=10 mice per group for control and TRT monotherapy groups, and N=7 for ICI and combination groups.

FIG. 5: Plot of tumor volume by treatment group as described in Example 2. N=10 mice per group for control and TRT monotherapy groups, and N=7 for ICI and combination groups.

FIG. 6: Plot of tumor volume for individual Renca-hCAIX tumour-bearing mice. Individual mice are shown in gray and mean is shown in black. (A) control; (B) 12 MBq 177Lu-hG250; (C) 18 MBq 177Lu-hG250; (D) 24 MBq 177Lu-hG250; (E) immune checkpoint inhibitor, aPD1 and aCTLA4; (E) ICI+TRT, 18 MBq 177Lu-hG250+aPD1+aCTLA4—as described in Example 2. N=10 mice per group for control and TRT monotherapy groups, and N=7 for ICI and combination groups.

FIG. 7: Experimental design for low dose combination TRT+ICI therapy (as described in Example 3).

FIG. 8: Plot of tumor volume by treatment group as described in Example 3. In (A) individual mice are shown in gray and mean of the group is shown in black. In (B) the means of tumour volumes are plotted. (C) Plots area under tumor growth curves corrected for lifetime in Renca-hCAIX tumour-bearing mice following treatment described in the legend. N=10 mice per group.

FIG. 9: (A) Survival curves for Renca-hCAIX tumour-bearing mice by treatment group as described in Example 3. (B) shows reason for euthanization of mice in each treatment group. (C) shows how many animals had a complete tumour regression, and of those animals with complete tumour regressions, how many rejected a subsequent implantation with either Renca cells or Renca-hCAIX cells. N=10 mice per group.

FIG. 10: Experimental design for low dose combination TRT+ICI therapy in the CT26-huCAIX model.

FIG. 11: (A) Plot of tumor volume by treatment group in CT26-hCAIX tumour-bearing mice. (A) individual mice are shown in gray and mean of the group is shown in black. (B) Plots area under tumor growth curves corrected for lifetime in CT26-hCAIX tumour-bearing mice following treatment described in the legend. N=10 mice per group.

FIG. 12: (A) Survival curves for CT26-hCAIX tumour-bearing mice by treatment group. (B) shows percentage of animals that had a complete tumour regression, and of those animals with complete tumour regressions, how many rejected a subsequent implantation with either CT26 cells or CT26 hCAIX cells. N=10 mice per group to start the experiment. N=10 mice per group.

FIG. 13: 177-Lu-hG250 treatment induces expression of several genes involved in anti-tumour immunity and that may facilitate checkpoint inhibition. Tumour gene expression profiling of Renca-hCAIX tumour bearing mice was performed using the nanostring platform. TRT groups were treated with 177-Lu-hG250, 12MBq of radiation. Tumours were harvested 7 days later for gene expression analysis. Graphs are derived from a list of genes passing a filter of at least 1.5 fold differential expression and p value<0.05. N=3 mice per group. Expression values represent the mean subtracted normalized log 2 values.

FIG. 14: Schematic of experimental design for determination of tumour gene expression profiling following low dose TRT+ICI therapy (as described in Example 5).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present invention relates to the combination of immune checkpoint inhibitor (ICI) therapy with targeted radionuclide therapy (TRT). Surprisingly, the inventors have found that the combination of these separate therapeutic approaches results in improved therapeutic outcomes relative to either immune checkpoint inhibitor therapy or targeted radionuclide therapy alone.

The present invention is directed in part to the identification of a new approach for reducing the toxicity of radioimmunoconjugates for use in radioimmunotherapy. In particular, the method of the present invention enables the use of relatively lower doses of radioimmunoconjugates to achieve comparable, and in some cases superior, therapeutic outcomes.

In addition, the invention is also in part directed to improved immune checkpoint inhibitor therapies.

As previously outlined above, radiation damage to healthy tissues and cells is a major problem associated with radioimmunotherapy. The toxicity severely limits the radiation dosage of RAIT and reduces the effectiveness of tumor treatment. However, the present inventors have realised that lower doses of radionuclide can be used to achieve at least comparable (and in preferred situations, improved) outcomes.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

### CAIX

As used herein, carbonic anhydrase is also known as: CA-IX, CA9, CAIX, Carbonate dehydratase IX, Carbonic anhydrase 9, Carbonic anhydrase IX, carbonic dehydratase, G250, Membrane antigen MN, P54/58N, pMW1, RCC-associated antigen G250, RCC-associated protein G250, and Renal cell carcinoma-associated antigen G250.

Cancer cells primarily express the plasma-membrane-associated CA isoforms CAIX and CAXII, as well as intracellular CAs such as CAI and CAII. Amongst the cancer-related CAs, CAIX has gained most attention, since expression of this isoform in healthy tissue is restricted to epithelial cells in the stomach and gut, but is strongly upregulated in renal cancers.

CAIX, the expression of which is under control of the hypoxia-inducible factor 1 (HIF-1), is predominantly located in chronically hypoxic tumour regions. However, CAIX can also be found in mild hypoxic or even normoxic regions, since the expression of CAIX can be activated by components of the mitogen-activated protein kinase (MAPK) pathway.

### Agents for Binding to CAIX

The agent for binding to CAIX and for use in accordance with the methods of the invention, may be any compound that specifically recognises or binds to CAIX, mediates its activity by binding to CAIX or a fragment or splice variant thereof; irreversibly binds at the entrance to the active site, and/or inhibits CAIX by coordinating to the zinc ion at the active site of CAIX.

Preferably the agent for binding to CAIX specifically interacts with a CAIX polypeptide. Specifically interacting with (e.g. recognising or binding to) means that the agent e.g. antibody, has a greater affinity for CAIX compared to other polypeptides. In one embodiment the agent interacts with (i.e. binds to or recognises) or modulates the activity of CAIX polypeptide and/or mediates an antibody dependent cell cytotoxicity (ADCC) and/or complement mediated cytotoxicity (CDC). Thus, according to one embodiment, the agent is a CAIX inhibitor. Said CAIX inhibitor may act on the protein level or the nucleic acid level. Examples for CAIX inhibitors e.g. acting on the protein level include but are not limited to peptides and anti-CAIX antibodies as well as functional fragments of those antibodies or small organic molecules, preferably having a molecular weight below 500 g/mol.

Examples of anti-CAIX antibodies or antibodies for binding to CAIX are described in EP 637 336, WO 93/18152, WO 95/34650, WO 00/24913, WO 02/063010, WO 04/025302, WO 05/037083, WO 2011/139375, U.S. Pat. No. 11,629,199, Murri-Plesko et al., Eur J Pharmacol 201 1, 657: 173-183.

Examples of small organic molecules for binding to CAIX include but are not limited to sulphonamides, heteroaromatic sulphonamides, sulfamates, coumarins and thiocoumarins and BAY-79-4620. Examples for inhibitors acting on the nucleic acid level are siRNA molecules, ribozymes and/or antisense molecules.

As used herein, the term “specifically binds” or “binds specifically” shall be taken to mean that an agent for use according to the invention, reacts or associates more frequently, more rapidly, with greater duration and/or with greater affinity with a CAIX or cell expressing same than it does with alternative antigens or cells. For example, an antigen binding protein that binds to CAIX with materially greater affinity (e.g., 1.5 fold or 2 fold or 5 fold or 10 fold or 20 fold or 40 fold or 60 fold or 80 fold to 100 fold or 150 fold or 200 fold) than it does to other antigens.

Methods for assessing binding to a protein (eg CAIX) are known in the art, e.g., as described in Scopes (In: Protein purification: principles and practice, Third Edition, Springer Verlag, 1994). Such a method generally involves immobilizing the agent (eg antibody) and contacting it with labeled target (in the case of an antibody, the antigen). Following washing to remove non-specific bound protein, the amount of label and, as a consequence, bound antigen is detected. Of course, the antigen binding site can be labeled and the antigen immobilized. Panning-type assays can also be used. Alternatively, or additionally, surface plasmon resonance assays can be used.

Other standard methods for assessing binding of to a target, such as CAIX, are also known in the art.

In any embodiment, the agent for binding to CAIX is the small molecule SLC-0111 (CAS 178606-66-1), SLC-149 (as described in EP 3317255 B1, incorporated herein by reference), SLC-0121 or SLC-101.

In any embodiment, the agent for binding to CAIX is the small molecule/contrast dye PMI-05 (as described in US2019/0192699A1, incorporated herein by reference).

In any embodiment, the agent for binding to CAIX is the small molecule sulfamide-nitroimidazole (as described in Rami et al., (2013), J. Med. Chem, 56: 8512-8520, incorporated herein by reference).

In any embodiment, the agent for binding to CAIX is the small molecule JS-403 (as described in WO 2010/089752 A1, incorporated herein by reference).

In any embodiment, the agent for binding to CAIX is the small molecule UB-TT220 (as described in WO 2022/015955 A1, incorporated herein by reference).

In any embodiment, the agent for binding to CAIX is the small molecule ^{99m}Tc-HEHEHE-Z09781 (Kim et al., (2017) Advanced Science, 4:1600471; Gebauer and Skerra (2009) Current Opin in Chem Biol, 13(3):245-55; Schardt et al., (2017) Mol Pharmaceutics, 14(4):1047-56; Tolmachev et al., (2008) Bioconjugate Chem, 19(8):1579-87; Liu et al., (2022) Analytical and Bioanalytical Chemistry, 414:1095-1104; Grindel et al., (2022) ACS Chem Biol, 17(6): 1543-55, incorporated herein by reference), ^{99m}Tc-MIP-1486, ^{99m}Tc-MIP-1490 (4-(2-bis((1-(2-((1,5-dicarboxy-3-(2-carboxyethyl)pentan-3-yl)amino)-2-oxoethyl)-1H-imidazol-2-yl)methyl)amino-X)benzenesulfonamide where X=ethyl) or ^{99m}Tc-MIP-1504 (4-(2-bis((1-(2-((1,5-dicarboxy-3-(2-carboxyethyl)pentan-3-yl)amino)-2-oxoethyl)-1H-imidazol-2-yl)methyl)amino-X)benzenesulfonamide where X=n-butyloxy) (Hillier et al., (2012) Journal of Nuclear Medicine, 53(s1):217, incorporated herein by reference)

In any embodiment, the agent for binding to CAIX is the small molecule PHC-102 (as described in WO 2015/114171 A1; WO 2018154517 A1; US 2014/0357650 A1; WO 2015/114171 A1, incorporated herein by reference)

In any embodiment, the agent for binding to CAIX is a peptide. As used herein, a peptide will be understood to comprise a chain of more than 1 amino acid residues. Typically, a peptide may comprise from about 2 to 30 amino acids, e.g. from about 5 to 30, from about 10 to 30, from about 2 to 25, from about 5 to 25, from about 10 to 25, or from about 10 to 20 amino acids. A peptide may have a length of at least 11, 12, 13, 14, 15, 16, 17, 18, 19, 20, 21, 22, 23, 24, 25 or 30 amino acids. Typically a peptide is no longer than about 40 amino acids, e.g. no longer than about 35, 30, 25, 20, 17, 15, 14, 13, 12, 11 or 10 amino acids.

In any embodiment, the agent for binding to CAIX is the peptide 3B-301 (also known as Debio 0228; Queen et al., (2018) Int J of Biol Macromol, 106:840-850; Eldehna et al., (2019) Bioorganic Chem, 90:103102; Lavecchia et al., (2011) Carbohydrate Res, 346(3):442-48; Krymov et al., (2022) Eur J of Medicinal Chem, 228:113997; Supuran (2008) BJI Int, 101(s4):39-40; Koyuncu et al., (2019) J of Enzyme Inhibition and Medicinal Chem, 34(1):703-11; Kumar et al., (2017) Eur J of Medicinal Chem, 136:52-62, incorporated herein by reference) or 3B-302.

In any embodiment, the agent for binding to CAIX is the peptide CAIX-P1, having the amino acid sequence YNTNHVPLSPKY (as described in Askoxylakis et al., (2010), PLoS One, 5(12): e15962), optionally wherein the peptide is labelled with 125I or 131I, for enabling detection thereof (although it will be appreciated that any suitable radiolabel or other detectable moiety may be used). As used herein, the term ‘theranostic’ refers to the ability of compounds/materials to be used for diagnosis as well as for therapy. The term “theranostics reagent” relates to any reagent which is both suitable for detection, diagnostic and/or the treatment of a disease or condition of a patient. The aim of theranostic compounds/materials is to overcome undesirable differences in biodistribution and selectivity, which can exist between distinct diagnostic and therapeutic agents. With a theranostic pair, the theranostic compound containing the imaging radionuclide is first administered to the patient in order to identify the disease or to locate the affected area in the body. Once identified/located, the disease can be treated by administering the theranostic compound containing the therapeutic radionuclide in a target specific way as the biodistribution of the imaging and therapy radionuclides are the same.

### Antibodies for Binding to CAIX

According to especially preferred embodiments, the agent for binding to CAIX is preferably an anti-CAIX antibody and/or a functional fragment of such an antibody. The fragment of the anti-CAIX antibody may have essentially the same CAIX-binding and/or inhibiting activity as the full-length anti-CAIX antibody and/or is an epitope-binding fragment of the anti-CAIX antibody.

Reference herein to an antibody or antigen binding fragment thereof that “binds to” carbonic anhydrase IX (CAIX) provides literal support for an antibody or fragment thereof that “binds specifically to” or “specifically binds to” CAIX.

The antibody and/or the antibody fragment thereof may be selected from the group consisting of polyclonal antibodies, monoclonal antibodies, antigen-binding fragments thereof such as F(ab′)2, Fab′, scFv, dsFv and chimerized, humanized and fully human variants thereof. The antibody may be multivalent, or multivalent and multispecific. The antibody may include human constant regions of IgG1, IgG2a, IgG3, or IgG4.

According to a further preferred embodiment, an anti-CAIX antibody or epitope-binding fragment thereof for use according to the invention, binds to the amino acid sequence LSTAFARV and/or ALGPGREYRAL.

In any embodiment, the agent for binding to CAIX is in the form of the antibody BAY-794620, or an antigen binding fragment thereof (as described in WO 2003/100029 A2; WO 2003/033674 A2; Theiner et al., (2021) Tierarztl Prax Ausg G Grosstiere Nutztiere, 49(6): 392-402; Kimani et al., (2011) Photochemistry and Photobiology, 88(1):175-87; NCT01065623 (v24, 30 Sep. 2014); NCT01028755 (v30, 19 Jan. 2015), incorporated herein by reference).

In any embodiment, the agent for binding to CAIX is in the form of the antibody SLC-0131, or an antigen binding fragment thereof.

In any embodiment, the agent for binding to CAIX is in the form of an antibody or antigen binding fragment thereof, as described in U.S. Pat. No. 11,629,199, incorporated herein by reference.

According to further particularly preferred embodiments, the agent for binding to CAIX is the antibody anti-G250 and/or an antigen-binding fragment thereof. Anti-G250 antibodies are, e.g., described in EP-B-0 637 336. The antibody or fragment thereof may be chimeric or humanised G250 antibody. In some embodiments, the antigen binding protein that binds to or specifically binds to CAIX is as described in any of WO 2002/062972 A2 (US 2004/0219633 A1), WO 2004/002526 A1 (U.S. Pat. No. 7,632,496 B2), WO 2006/002889 A2 (U.S. Pat. No. 7,691,375 B2), WO 2009/056342 A1 (US 2014/0017252 A1), WO 2011/032973 A1 (US 2012/0207672 A1), and WO 2014/128258 A1 (U.S. Pat. No. 10,620,208 B2), or WO 2021/000017 A1, the entire contents of each of these publications is incorporated herein by reference.

The antibodies for use in the present invention may be produced by any suitable method known in the art including but not limited by methods as described in PCT/EPO2/01282 and PCT/EPO2/01283, which are incorporated herein by reference.

An especially preferred antibody is cG250, preferably girentuximab (INN). Another especially preferred embodiment is the monoclonal antibody G250 produced by the hybridoma cell line DSM ACC 2526. The antibody cG250 is an IgG1 kappa light chain chimeric version of an originally murine monoclonal antibody mG250.

Variants of the original chimeric G250 (cG250) antibody are known, including WX-G250 and WX-G250RIT (131 iodine) (Janssen Global Services LLC).

In a particularly preferred embodiment, the antibody is ⁸⁹Zr-girentuximab (ie ⁸⁹Zr-cG250), ¹²³I-, ¹²⁴I-, or ¹³¹I-girentuximab, or ¹⁷⁷Lu-girentuximab.

In the context of the present invention, the antibodies for use according to the invention are particularly useful for inclusion in theranostic pairs, for example, where the antibody is conjugated to a radioisotope for imaging or diagnostic purposes, and the same antibody is conjugated with a different radioisotope or a cytotoxic agent which is suitable for therapy. The antigen binding domain of the antibody directs or targets the diagnostic radioisotope to the site of the tumor to facilitate diagnosis (including tumor distribution, tumor size, tumor density), while the same antigen binding domain of the antibody directs the radioisotope to the tumor for therapy.

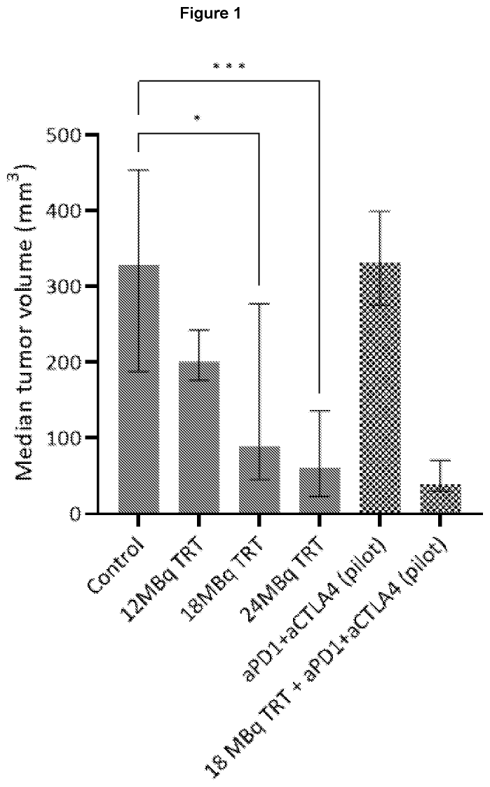

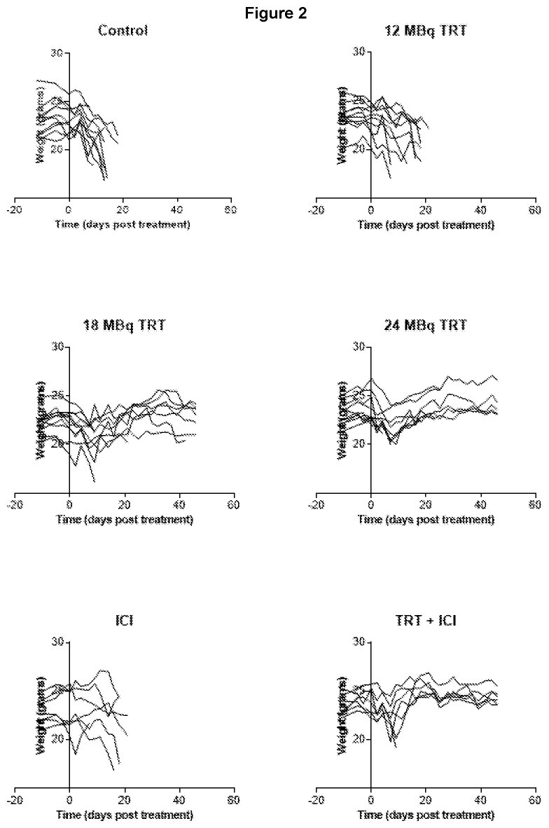

The term “Fc region”, sometimes referred to as “Fc” or “Fc domain”, as used herein refers the portion of an IgG molecule that correlates to a crystallizable fragment obtained by papain digestion of an IgG molecule. The Fc region consists of the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc region contains the entire second constant domain CH2 (residues 231-340 of human IgG1, according to the EU Index numbering system, also defined as residues 244 to 360 in the Kabat system) and the third constant domain CH3 (residues 341-447 EU Index/361-478 Kabat) (e.g., see SEQ ID NO 1 of WO2015175874 or FIG. 1C for the sequence of CH2 and SEQ ID NO: 2; FIG. 1D for the sequence of CH3, incorporated herein by reference; see also http://www.imgt.org/IMGTScientificChart/Numbering/Hu_lGHGnber.html #refs for a comparison of the numbering conventions used for various residues in the Fc region of immunoglobulins).

As used herein, the “EU index” or “EU numbering scheme” refers to the numbering of the EU antibody (Edelman et al., 1969, Proc Natl Acad Sci USA 63:78-85, hereby entirely incorporated by reference.) As used herein, the “Kabat system” refers to the Kabat Sequences of Proteins of Immunological Interest, National Institutes of Health, Bethesda, Md., 1987 and 1991. The skilled person will be able to readily determine whether a given amino acid sequence is numbered according to either EU or Kabat systems.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding site” may be used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (~50 to 70 kD) covalently linked and two light chains (~23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ~110 amino acids in length) and one or more constant domains at the C-terminus. The constant domain of the light chain (CL which is ~110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding protein for use according to the invention reacts or associates more frequently, more rapidly, with greater duration and/or with greater affinity with a particular antigen or cell expressing same than it does with alternative antigens or cells. For example, an antigen binding protein binds to CAIX with materially greater affinity (e.g., 1.5 fold or 2 fold or 5 fold or 10 fold or 20 fold or 40 fold or 60 fold or 80 fold to 100 fold or 150 fold or 200 fold) than it does to other antigens.

As used herein, the term “epitope” (syn. “antigenic determinant”) shall be understood to mean a region of a cell surface protein (such as CAIX) to which an antigen binding protein comprising an antigen binding domain of an antibody binds.

As used herein, the term “condition” refers to a disruption of or interference with normal function, and is not to be limited to any specific condition, and will include diseases or disorders.

As used herein, the terms “preventing”, “prevent” or “prevention” include administering an active species such as an antigen binding protein described herein or a checkpoint inhibitor to thereby stop or hinder the development of at least one symptom of a condition. This term also encompasses treatment of a subject in remission to prevent or hinder relapse.

As used herein, the terms “treating”, “treat” or “treatment” include administering an active species such as an antigen binding protein described herein or a checkpoint inhibitor to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

### Modified Antibodies

The present invention relates in part to the use of antibodies comprising modifications to IgG antibodies, which include one or more amino acid substitutions to a region of the antibody which reduces or abolishes the affinity of the antibody for FcRn, thereby reducing the serum half-life of the antibodies.

It will be understood that in accordance with the present invention, any antibody for which reduced serum half-life is desired can be modified according to the methods described herein.

In certain embodiments, the antibodies suitable for modification according the present invention, to reduce affinity for FcRn, are antibodies having one or more of the sequences as shown in Table 1.

The present invention also provides the use of an antigen binding protein having at least 80% identity to a sequence disclosed herein. In one example, an antigen binding protein for use according to the invention comprises sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% identical to a sequence disclosed herein.

Alternatively, or additionally, the antigen binding protein comprises a CDR (e.g., three CDRs) 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% identical to CDR(s) of a V_{H} or V_{L} as described herein according to any example.

The present invention also contemplates mutant forms of an antigen binding protein for use according to the present invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), #-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophilic indices are described in, e.g., U.S. Pat. No. 4,554,101.

In some embodiments the antigen binding protein comprises non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding protein. In another example, the mutation(s) occur within a CDR of an antigen binding protein.

Exemplary methods for producing mutant forms of an antigen binding protein include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1 Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the antigen binding protein for use according to the invention will be apparent to the skilled artisan and/or described herein, e.g., antigen binding. For example, methods for determining antigen binding, competitive inhibition of binding, affinity, association, dissociation and therapeutic efficacy are described herein.

### Constant Regions

The present invention also provides the use of antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

### Preferred Modifications

The present invention also contemplates modifications to an antibody or antigen binding protein, for use according to the present invention, comprising an Fc region or constant region.

The neonatal Fc-receptor (FcRn) is important for the metabolic fate of antibodies of the IgG class in vivo. The FcRn functions to salvage IgG from the lysosomal degradation pathway, resulting in reduced clearance and increased half-life. It is a heterodimeric protein consisting of two polypeptides: a 50 kDa class I major histocompatibility complex-like protein (a-FcRn) and a 15 kDa p2-microglobulin (β2ηι). FcRn binds with high affinity to the CH2-CH3 portion of the Fc-region of an antibody of the class IgG. The interaction between an antibody of the class IgG and the FcRn is pH dependent and occurs in a 1:2 stoichiometry, i.e. one IgG antibody molecule can interact with two FcRn molecules via its two heavy chain Fc-region polypeptides (see e.g. Huber, A. H., et al, J. Mol. Biol. 230 (1993) 1077-1083).

Thus, an IgG's in vitro FcRn binding properties/characteristics are indicative of its in vivo pharmacokinetic properties in the blood circulation. In the interaction between the FcRn and the Fc-region of an antibody of the IgG class different amino acid residues of the heavy chain CH2- and CH3-domain are participating.

Different mutations that influence the FcRn binding and therewith the half-live in the blood circulation are known. Fc-region residues critical to the mouse Fc-region-mouse FcRn interaction have been identified by site-directed mutagenesis (see e.g. Dall'Acqua, W. F., et al. J. Immunol 169 (2002) 5171-5180). Residues Ile253, His310, His433, Asn434 and His435 (numbering according to EU index numbering system) are involved in the interaction (Medesan, C, et al., Eur. J. Immunol. 26 (1996) 2533-2536; Firan, M., et al, Int. Immunol. 13 (2001) 993-1002; Kim, J. K., et al, Eur. J. Immunol. 24 (1994) 542-548). (Using the Kabat system, the relevant residues are Ile266, His329, His464, Asn465 and His466). Residues Ile253, His310, and His435 were found to be critical for the interaction of human Fc-region with murine FcRn (Kim, J. K., et al, Eur. J. Immunol. 29 (1999) 2819-2885).

More specifically, the antibody may comprise one or more amino acid substitutions that decrease the half-life of the protein. For example, the antibody comprises a Fc region comprising one or more amino acid substitutions that decrease the affinity of the Fc region for the neonatal Fc region (FcRn).

The present invention also provides use of an antibody having a constant region substantially identical to a naturally occurring class IgG antibody constant region wherein at least one amino acid residue selected from the group consisting of residues His310, His435, and Ile253 is different from that present in the naturally occurring class IgG antibody, thereby altering FcRn binding affinity and/or serum half-life of said antibody relative to the naturally occurring antibody. In preferred embodiments, the naturally occurring class IgG antibody comprises a heavy chain constant region of a human IgG1, IgG2, IgG2M3, IgG3 or IgG4 molecule.

Also in preferred embodiments, amino acid residue 310 or residue 435 from the heavy chain constant region of the antibody having a constant region substantially identical to the naturally occurring class IgG antibody is any amino acid that is not histidine and which reduces the affinity of the constant region for FcRn. For example, the amino acid at residue 310 or 435 may be alanine, glutamic acid, aspartic acid, leucine, isoleucine, arginine, proline, glutamine, methionine, serine, threonine, lysine, asparagine, phenylalanine, tyrosine, tryptophan, cysteine, valine or glycine.

Preferably, the residue at position 310 is selected from alanine, or glutamic acid or glutamine; or amino acid residue 435 from the heavy chain constant region is selected from arginine, glutamine or alanine. In other preferred embodiments, the antibody having a constant region substantially identical to a naturally occurring class IgG antibody has an alanine residue at position 310 and glutamine residue at position 435.

In a preferred embodiment of the present invention, the binding affinity for FcRn and/or the serum half-life of the modified antibody is decreased by at least about 30%, 50%, 80%, 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, or 100-fold. In a preferred embodiment of the present invention, the binding affinity for FcRn and/or the serum half-life of said modified antibody is reduced by at least about 20%, 30%, 40%, 50%, 60%, 70%, 80%, 85%, 90%, 95%, 97%, 98%, or 99%.

In addition, the antibodies for use according to the present invention may comprise one or more mutations which modify the affinity of the antibodies for any one or more Fc gamma receptors.

In some embodiments, the Fc region of the constant region retains the ability to induce effector functions. In one example, the Fc region of the constant region contains one or more amino acid substitutions that modulate effector function, including increasing effector function compared to a wild-type IgG.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the amino acid substitution that modifies that ability of the antibody to induce effector function is an amino acid substitution at residue Ile253 from the heavy chain constant region. In one example, the substitution is to any amino acid selected from be alanine, glutamic acid, aspartic acid, leucine, isoleucine, arginine, proline, glutamine, methionine, serine, threonine, lysine, asparagine, phenylalanine, tyrosine, tryptophan, cysteine, valine or glycine, wherein the substitution reduces the ability of the antibody to induce effector function. In preferred embodiments, the substitution from Ile at residue 253 is to arginine, proline, or aspartate, more preferably alanine.

In one example, the Fc region is an IgG4 Fc region (i.e., from an IgG4 constant region), e.g., a human IgG4 Fc region. Sequences of suitable IgG4 Fc regions will be apparent to the skilled person and/or available in publicly available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system. In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat (or Glu216 to Pro230 using the EU index). Hinge regions of other IgG isotypes may be aligned with the IgG1 sequence by placing the first and last cysteine residues forming inter-heavy chain disulphide (S-S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Antibody Production

Preferably, an antigen binding protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the not promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FIO (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where an antigen binding protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Linking of Radioisotopes to Antibodies

In any embodiment of the invention, the antibodies herein described may be directly or indirectly linked to a therapeutic agent, Preferably, the therapeutic agent is a radioisotope.

As used herein, the term “radioisotope” is used interchangeably with “radionuclide”.

Examples of suitable isotopes include: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), iodine-123, -124, -125 or -131 (¹²³I, ¹²⁴I, ¹²⁵I, ¹³¹I) (123 I), lead-212 (²¹²Pb), lutetium-177 (¹⁷⁷Lu), radium-223 and radium-224 (²²³Ra, ²²⁴Ra), rhenium-186 and rhenium-188 (¹¹⁶Re and ¹⁸⁸Re), samarium-153 (¹⁵³Sm), scandium-47 (⁴⁷Sc), strontium-90 (⁹⁰Sr), terbium-149 and terbium-161 (¹⁴⁹Tb and ¹⁶¹Tb) and yttrium-90 (⁹⁰Y).

In some embodiments, the antigen binding protein is conjugated with a radioisotope capable of therapy and diagnosis, which may be useful to treat cancer in a subject and also assess spread of the cancer by binding CAIX, or size of a tumor expressing CAIX being treated. The skilled person will be familiar with which of the therapeutic radioisotopes may also be used for imaging and which imaging techniques may be used.

It will be understood that the isotopes may be conjugated to the antibodies described herein directly (via a chelating agent or prosthetic group or linker) or indirectly via binding to single or multiple amino acid residues in the antibody (e.g. halogenation of tyrosine residues).

In alternative embodiments, chelating agents or linkers may be used in order to conjugate the radioisotope to the antibody. In one example, the antibodies can be conjugated to a chelating moiety, selected from the group consisting of: TMT (6,6″-bis[N,N″,N″′-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N″-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl)acetamide), CB-DO2A (4,10-bis(carboxymethyl)-1,4,7,10-tetraazabicyclo[5.5.2]tetradecan), NOTA (1,4,7-triazacyclononane-triacetic acid) Diamsar (3,6,10,13,16,19-hexaazabicyclo [6,6,6]eicosane-1,8-diamine), DTPA (Pentetic acid or diethylenetriaminepentaacetic acid), CHX-A″-DTPA ([(R)-2-Amino-3-(4-isothiocyanatophenyl)propyl]-trans-(S,S)-cyclohexane-1,2-diamine-pentaacetic acid), TETA (1,4,8,11-tetraazacyclotetradecane-1,4,8), 11-tetraacetic acid, Te2A (4,11-bis(carboxymethyl)-1,4,8,11-tetraazabicyclo[6.6.2]hexadecane), HBED, DFO (Desferrioxamine), DFOsq (DFO-squaramide) and HOPO (3,4,3-(LI-1,2-HOPO), a chelator as described in WO 2022/133537 (incorporated herein by reference) or other chelating agent as described herein.

Chelators with radiometals and other halogenated radioisotopes may be bound to the antibodies via one or more amino acid residues or reactive moieties in the antibody, including but not limited to one or more lysine residues, tyrosine residues or thiol moieties.

In another example, the modified antibody is conjugated to a bifunctional linker, for example, bromoacetyl, thiols, succinimide ester, TFP ester, a maleimide, or using any amine or thiol-modifying chemistry known in the art.

The skilled person will be familiar with standard methods for conjugating chelating agents to antibodies and derivatives or fragments thereof. In addition, the skilled person will be familiar with approaches for selecting a relevant chelating agent for pairing with a radiometal, for example as described in Chem. Soc. Rev., 2014, 43, 260, incorporated herein by reference.

### Assaying Activity of an Antigen Binding Protein

### Binding to CAIX

It will be apparent to the skilled artisan from the disclosure herein that the preferred antigen binding proteins for use according to the present invention bind to CAIX. Methods for assessing binding to a protein are known in the art, e.g., as described in Scopes (In: Protein purification: principles and practice, Third Edition, Springer Verlag, 1994). Such a method generally involves immobilizing the antigen binding protein and contacting it with labeled antigen. Following washing to remove non-specific bound protein, the amount of label and, as a consequence, bound antigen is detected. Of course, the antigen binding protein can be labeled and the antigen immobilized. Panning-type assays can also be used. Alternatively, or additionally, surface plasmon resonance assays can be used.

### Therapeutic Methods

The antibodies for use according to the present invention are useful for treating a number of conditions requiring treatment by radioimmunotherapy. Typically, such conditions include cancer.

Exemplary cancers include cystic and solid tumors, bone and soft tissue tumors, including tumors in anal tissue, bile duct, bladder, blood cells, bowel, brain, breast, carcinoid, cervix, eye, esophagus, head and neck, kidney, larynx, leukemia, liver, lung, lymph nodes, lymphoma, melanoma, mesothelioma, myeloma, ovary, pancreas, penis, prostate, skin (e.g. squamous cell carcinoma), sarcomas, stomach, testes, thyroid, vagina, vulva. Soft tissue tumors include Benign schwannoma Monosomy, Desmoid tumor, lipo-blastoma, lipoma, uterine leiomyoma, clear cell sarcoma, dermatofibrosarcoma, Ewing sarcoma, extraskeletal myxoid chondrosarcoma, liposarcooma myxoid, Alveolar rhabdomyosarcoma and synovial sarcoma. Specific bone tumors include nonossifying fibroma, unicameral bone cyst, enchon-droma, aneurismal bone cyst, osteoblastoma, chondroblastoma, chondromyxofibroma, ossifying fibroma and adamantinoma, Giant cell tumor, fibrous dysplasia, Ewing's sarcoma eosinophilic granuloma, osteosarcoma, chondroma, chondrosarcoma, malignant fibrous histiocytoma and metastatic carcinoma. Leukemias include acute lymphoblastic, acute myeloblastic, chronic lymphocytic and chronic myeloid.

Other examples include breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small round cell tumor, squamous cell cancer, melanoma, atypical fibroxanthoma, seminomas, nonseminomas, stromal leydig cell tumors, Sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor.

In some embodiments, the cancer is metastatic cancer. The primary source for the metastatic cancer may be any cancer type known in the art, including those described herein.

Preferably, the antigen binding proteins for use according to the present invention are useful for treating cancer that are characterised by the presence of CAIX. For example, the antibodies that bind to CAIX are useful for treating cancers characterised by increased expression of CAIX, including renal cell carcinoma.

### Antibody Binding Domain Containing Proteins

### Single-Domain Antibodies

In some examples, an antigen binding protein for use according to the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, MA; see, e.g., U.S. Pat. No. 6,248,516).

### Diabodies, Triabodies, Tetrabodies

In some examples, a protein for use according to the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}—X—V_{H} or V_{H}—X—V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding domain, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present invention also contemplates use of a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present invention encompasses use of a dimeric scFv, i.e., a protein comprising two scFV molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “V_{HH} domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Proteins Comprising Antigen Binding Domains Thereof

The present invention also contemplates use of other antibodies and proteins comprising antigen-binding domains thereof, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### Administration Routes

In some examples, agent as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an agent as described herein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The agents for use according to the invention are particularly useful for parenteral administration, such as intravenous administration or administration into a body cavity or lumen of an organ or joint. In especially preferred embodiments, an antigen binding protein for use according to the invention is preferably formulated for intravenous, intraperitoneal, subcutaneous, intramuscular or intra-tumoral administration.

The compositions for administration will commonly comprise a solution of an antigen binding protein dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding protein of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Non-aqueous vehicles such as mixed oils and ethyl oleate may also be used. Liposomes may also be used as carriers. The vehicles may contain minor amounts of additives that enhance isotonicity and chemical stability, e.g., buffers and preservatives.

### Immune Checkpoint Inhibitors

### Checkpoint Inhibitors

A “checkpoint inhibitor” inactivates a protein in an inhibitory checkpoint pathway of an immune response.

In the context of cancer, checkpoint inhibitors regulate the immune system by blocking proteins that stop the immune system from attacking cancer cells. In particular, they control how detection-evading cancer cells and T-cells interact so that T-cells can recognize tumor cells and mount an appropriate immune response against them. Non-limiting examples of checkpoint inhibitors that may be used in accordance with the methods described herein include inhibitors that target PD-1 (programmed cell death protein 1), CTLA-4 (cytotoxic T lymphocyte associated protein 4) and PD-L1 (programmed death ligand 1). A skilled person will understand that CTLA-4 and PD-1 are found on T cells and that PD-L1 is expressed on cancer cells and/or suppressive immune cells. Other non-limiting examples include PD-L2, TIM3, LAG3, CEACAM (e.g., CEACAM-1, CEACAM-3 and/or CEACAM-5), VISTA, BTLA, TIGIT, LAIR1, CD160, 2B4, CD80, CD86, B7-H3 (CD276), B7-H4 (VTCN1), HVEM (TNFRSF14 or CD107), KIR, A2aR, MHC class I, MHC class II, GAL9, adenosine, and TGF-beta.

Other immune checkpoints include Indoleamine 2,3-dioxygenase (IDO) and CSF-R1. Inhibitors of those proteins are also contemplated as immune checkpoint inhibitors for use in the invention.

“Programmed Death-1 (PD-1)” refers to an immunoinhibitory receptor belonging to the CD28 family. PD-1 is expressed predominantly on activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

Upon binding of PD-1 to programmed cell death ligand 1 (PD-L1), an immune reaction is turned off so as to prevent T-cells from damaging or killing the cell. In the context of cancer, cancer cells can be covered with PD-L1 proteins to camouflage themselves as healthy cells thus avoiding an immune response. Programmed Death Ligand-1 (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (HPD-L1), variants, isoforms, and species homologs of hPD-L1, and analogs having at least one common epitope with hPD-L1. The complete hPD-L1 sequence can be found under GenBank Accession No. Q9NZQ7.

Cytotoxic T-Lymphocyte Antigen-4 (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

Any of the checkpoint inhibitors described herein may be administered in the form of an antibody. An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs can mediate the binding of the immunoglobulin to host tissues or factors, including various cells of the immune system (e.g., effector cells) and the first component (Clq) of the classical complement system.

The term “antibody” includes, by way of example monoclonal and polyclonal Abs; chimeric and humanized Abs; human or nonhuman Abs; wholly synthetic Abs; and single chain Abs. A nonhuman Ab can be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “isolated antibody” refers to an Ab that is substantially free of other Abs having different antigenic specificities (e.g., an isolated Ab that binds specifically to PD-1 is substantially free of Abs that bind specifically to antigens other than PD-1). An isolated Ab that binds specifically to PD-1 can, however, have cross-reactivity to other antigens, such as PD-1 molecules from different species. Moreover, an isolated Ab can be substantially free of other cellular material and/or chemicals. The term “monoclonal antibody” (mAb) refers to a non-naturally occurring preparation of Ab molecules of single molecular composition, i.e., Ab molecules whose primary sequences are essentially identical, and which exhibits a single binding specificity and affinity for a particular epitope. A mAb is an example of an isolated Ab. mAbs can be produced by hybridoma, recombinant, transgenic or other techniques known to those skilled in the art.

A “human” antibody (huMAb) refers to an Ab having variable regions in which both the framework and CDR regions are derived from human germline immune globulin sequences. Furthermore, if the Ab contains a constant region, the constant region also is derived from human germline immunoglobulin sequences. The human Abs described herein can include amino acid residues not encoded by human germline immunoglobulin sequences (e.g., mutations introduced by random or site-specific mutagenesis in vitro or by somatic mutation in vivo). However, the term “human antibody,” as used herein, is not intended to include Abs in which CDR sequences derived from the germline of another mammalian species, such as a mouse, have been grafted onto human framework sequences. The terms “human” Abs and “fully human Abs and are used synonymously.

A “humanized antibody” refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible (including variations as a result of codon degeneracy leading to synonymous codons) as long as they do not abrogate the ability of the Ab to bind to a particular antigen. A “humanized” Ab retains an antigenic specificity similar to that of the original Ab.

A “chimeric antibody” refers to an Ab in which the variable regions are derived from one species and the constant regions are derived from another species, such as an Ab in which the variable regions are derived from a mouse Ab and the constant regions are derived from a human Ab.

An “anti-antigen” Ab refers to an Ab that binds specifically to the antigen. For example, an anti-PD-1 Ab binds specifically to PD-1 and an anti-CTLA-4 Ab binds specifically to CTLA-4.

An “antigen-binding portion” of an Ab (also called an “antigen-binding fragment”) refers to one or more fragments of an Ab that retain the ability to bind specifically to the antigen bound by the whole Ab.

Examples of immune checkpoints and antibody inhibitors that target those checkpoints include anti-CTLA-4 (e.g., Ipilimumab, Tremelimumab, KAHR-102), anti-TIM3 (e.g., F38-2E2. ENUM005), anti-LAG3 (e.g., BMS-986016, IMP701. IMP321, C9B7W), anti-KIR (e.g., Lirilumab, IPH2101, IPH4102), anti-PD-1 (e.g., Nivolumab, Pidilizumab, Pembrolizumab, BMS-936559, atezolizumab, Lambrolizumab, MK-3475. AMP-224, AMP-514, STI-A1110, TSR-042), anti-PD-L1 (e.g., KY-1003 (EP20120194977), MCLA-145, atezolizumab. BMS-936559, MEDI-4736, MSB0010718C, AUR-012, STI-A1010, MPDL3280A, AMP-224, Dapirolizumab pegol (CDP-7657), MEDI-4920), anti-CD73 (e.g., AR-42 (OSU-HDAC42, HDAC-42, AR42, AR 42, OSU-HDAC 42, OSU-HDAC-42, NSC D736012, HDAC-42, HDAC 42, HDAC42, NSCD736012, NSC-D736012), MEDI-9447), anti-B7-H3 (e.g., MGA271, DS-5573a, 8H9), anti-CD47 (e.g., CC-90002, TTI-621, VLST-007), anti-BTLA, anti-VISTA, anti-A2aR, anti-B7-1, anti-B7-H4, anti-CD52 (such as alemtuzumab), anti-IL-10, anti-IL-35, anti-TGF-β (such as Fresolumimab), anti-CSF1R (e.g., FPA008), anti-NKG2A (e.g., monalizumab), anti-MICA (e.g., IPH43), and anti-CD39.

### Anti-PD-1 Antibodies and Anti-PD-L1 Antibodies

Examples of suitable PD-1 inhibitors that may be used in accordance with the invention include Keytruda (pembrolizumab), Opdivo (nivolumab), AGEN 2034, BGB-A317, BI-754091, CBT-501 (genolimzumab), MED10680, MGA012, PDR001, PF-06801591, REGN2810 (SAR439684), and TSR-042 or those that are disclosed in U.S. Pat. No. 8,008,449. Other anti-PD-1 mAbs have been described in, for example, U.S. Pat. Nos. 6,808,710, 7,488,802, 8,168,757 and 8,354,509, and PCT Publication No. WO 2012/145493 (incorporated herein by reference).

Nivolumab (also known as “Opdivo®”; formerly designated 5C4, BMS-936558, MDX-1106, or ONO 538) is a fully human IgG4 (S228P) PD-1 immune check point inhibitor Ab that selectively prevents interaction with PD-1 ligands (PD-L1 and PD-L2), thereby blocking the down-regulation of antitumor T-cell functions (U.S. Pat. No. 8,008,449, incorporated herein by reference).

Pembrolizumab (also known as “Keytruda®”, lambrolizumab, and MK-3475) is a humanized monoclonal IgG4 antibody for binding to human cell surface receptor PD-1 (programmed death-1 or programmed cell death-1). Pembrolizumab is described for example, in U.S. Pat. Nos. 8,354,509 and 8,900,587, incorporated herein by reference). Pembrolizumab has been approved by the FDA for the treatment of relapsed or refractory melanoma.

Other suitable PD-1 inhibitors include Libtayo (cemiplimab), Blincyto (blinatumomab), Dostarlimab, Spartalizumab, Cetrelimab, Pidilizumab and BI-754091.

Anti-PD-1 Abs suitable for use in the disclosed methods or uses of the invention are Abs that bind to PD-1 with high specificity and affinity, block the binding of PD-L1 and or PD-L2, and inhibit the immunosuppressive effect of the PD-1 signalling pathway. In any of the compositions or methods disclosed herein, an anti-PD-1 antibody includes an antigen-binding portion or fragment that binds to the PD-1 receptor and exhibits the functional properties similar to those of whole Abs in inhibiting ligand binding and upregulating the immune system.

In certain embodiments, an anti-PD-1 antibody that may be used in accordance with the invention can be replaced with another PD-1 or anti-PD-L1 antagonist. For example, because an anti-PD-L1 antibody prevents interaction between PD-1 and PD-L1, thereby exerting similar effects to the signalling pathway of PD-1, an anti-PD-L1 antibody can replace the use of an anti-PD-1 antibody in the methods disclosed herein. In any embodiment, suitable PD-L1 inhibitors include Imfinzi (durvalumab or MED14736), Tecentriq (atezolizumab or MPDL3280A), Bavencio (avelumab; MSB0010718C), MS-936559 (12A4 or MDX-1105) and CX-072.

### Anti-CTLA-4 Antibodies

Anti-CTLA-4 antibodies that may be used in accordance with the instant invention bind to human CTLA-4 so as to disrupt the interaction of CTLA-4 with a human B7 receptor. It will be understood that because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Suitable CTLA-4 inhibitors that may be used in accordance with the invention include Yervoy (ipilimumab), Tremelimumab and AGEN 1884 or those disclosed in U.S. Pat. Nos. 6,984,720 and 7,605,238, incorporated herein by reference. Ipilimumab is a fully human, IgG1 monoclonal Ab that blocks the binding of CTLA-4 to its B7 ligands, thereby stimulating T cell activation. Tremelimumab is human IgG2 monoclonal anti-CTLA-4 antibody.

Immune checkpoint inhibitors may be administered in the form of a pharmaceutical composition, including in combination with any pharmaceutically acceptable excipient, carrier and/or diluent described herein. Typically, the immune checkpoint inhibitor is administered in a formulation as is known in the art.

### Dosages and Timing of Administration

In any aspect or embodiment of the present invention, therapeutically effective amounts of an antigen binding protein for binding to CAIX and an immune checkpoint inhibitor may be administered to a subject in need thereof.

The administration may be simultaneous, separate or sequential.

Administering refers to the physical introduction of a composition comprising a therapeutic agent to a subject, using any of the various methods and delivery systems known to those skilled in the art including those described herein. Pharmaceutical compositions may be formulated from active agents as described herein for any appropriate route of administration. Typically, in addition to the therapeutic agent (eg an antibody for binding to CAIX and/or checkpoint inhibitor), a pharmaceutical composition comprises a pharmaceutically acceptable excipient, carrier and/or diluent. Examples of suitable components for inclusion in a pharmaceutical composition are described in Martindale—The Extra Pharmacopoeia (Pharmaceutical Press, London 1993) and Martin (ed.), Remington's Pharmaceutical Sciences.

The phrase ‘therapeutically effective amount’ or ‘effective amount’ generally refers to an amount of an antibody against CAIX and/or checkpoint inhibitor of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”. In some embodiments, the antigen binding protein that binds to or specifically binds to carbonic anhydrase IX (CAIX) conjugated with a radionuclide and the immune checkpoint inhibitor are together administered to the subject in need thereof in a “therapeutically effective amount”. This therapeutically effective amount may comprise amounts of either or both the antigen binding protein or the immune checkpoint inhibitor that would by itself be less than a therapeutically effective amount. In some embodiments, the antigen binding protein that binds to or specifically binds to carbonic anhydrase IX (CAIX) conjugated with a radionuclide is administered in an amount that is less than a therapeutically effective amount absent the immune checkpoint inhibitor. In some embodiments, the immune checkpoint inhibitor is administered in an amount that is less than a therapeutically effective amount absent the antigen binding protein that binds to or specifically binds to carbonic anhydrase IX (CAIX) conjugated with a radionuclide. In some embodiments, the “therapeutically effective amount” for the combination of antigen binding protein that binds to or specifically binds to carbonic anhydrase IX (CAIX) conjugated with a radionuclide and the immune checkpoint inhibitor may be a synergistic amount. The synergistic amount may be synergistic relative to mono-therapy with either antigen binding protein that binds to or specifically binds to carbonic anhydrase IX (CAIX) conjugated with a radionuclide or the immune checkpoint inhibitor.

For instance, for the treatment of tumors, a therapeutically effective amount of the compounds or compositions described herein can inhibit tumor growth by at least about 10%, by at least about 20%, by at least about 30%, by at least about 40%, by at least about 50%, by at least about 60%, by at least about 70%, by at least about 80%, or by at least about 90% or more, relative to untreated subjects. Alternatively, the treatments described herein may cause complete regression of the tumor mass. In other embodiments of the invention, tumor regression can be observed and continue for a period of at least about 10 days, at least about 20 days, at least about 30 days, at least about 40 days, at least about 50 days or at least about 60 days, at least about 70 days, at least about 80 days, at least about 90 days, at least about 100 days or longer.

A therapeutically effective amount of a drug may also include a “preventative” or “prophylactically effective amount,” which is any amount of the antibody for binding to CAIX and/or checkpoint inhibitor is/are administered to a subject at risk of developing a cancer (eg a subject having a pre-malignant condition) or of suffering a recurrence of cancer, that inhibits the development or recurrence of the cancer. In certain embodiments, the prophylactically effective amount prevents the development or recurrence of the cancer entirely. “Inhibiting” or “preventing” the development or recurrence of a cancer means either lessening the likelihood of the cancer's development or recurrence, or preventing the development or recurrence of the cancer entirely.

Suitable dosages of an antigen binding protein for use according to the present invention and the immune checkpoint inhibitor will vary depending on the specific active agents selected, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

For administration of a checkpoint inhibitor including PD-1, PD-L1 or CTLA-4 inhibitors, the dosage can range from about 0.01 to about 20 mg/kg, about 0.1 to about 10 mg/kg, about 0.1 to about 5 mg/kg, about 1 to about 5 mg/kg, about 2 to about 5 mg/g, about 7.5 to about 12.5 mg/kg, or about 0.1 to about 30 mg/kg of the subject's body weight. For example, dosages can be about 0.1, about 0.3, about 1, about 2, about 3, about 5 or about 10 mg/kg body weight, or, about 0.3, about 1, about 2, about 3, or about 5 mg/kg body weight. The dosing schedule is typically designed to achieve exposures that result in sustained receptor occupancy (RO) based on typical pharmacokinetic properties of an Ab. An exemplary treatment regime entails administration about once per week, about once every 2 weeks, about once every 3 weeks, about once every 4 weeks, about once a month, about once every 3-6 months or longer. In certain embodiments, a checkpoint inhibitor is administered to the subject about once every 2 weeks. In other embodiments, the Ab is administered about once every 3 weeks. The dosage and scheduling can change during a course of treatment. For example, a dosing schedule for anti-PD-1 therapy can comprise administering the Ab: (i) about every 2 weeks in about 6-week cycles; (ii) about every 4 weeks for about six dosages, then about every three months; (iii) about every 3 weeks; (iv) about 3-about 10 mg/kg once followed by about 1 mg/kg every about 2-3 weeks. Considering that an IgG4 Ab typically has a half-life of 2-3 weeks, a dosage regimen for an anti-PD-1 Ab for use according to the invention comprises about 0.3-1 about 0 mg/kg body weight, 1-5 mg/kg body weight, or about 1-about 3 mg/kg body weight via intravenous administration, with the Ab being given every about 14-21 days in up to about 6-week or about 12-week cycles until complete response or confirmed progressive disease.

In some embodiments, the checkpoint inhibitor and/or antibody for binding to CAIX treatment disclosed herein, is continued for a minimum duration of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 18 months, at least about 24 months, at least about 3 years, at least about 5 years, or at least about 10 years. In some embodiments, the checkpoint inhibitor and/or antibody for binding to CAIX treatment disclosed herein, is continued for a maximum duration of up to about 18 months, 16 months, 14 months, 12 months, 10 months, 8 months, or 6 months. The treatment duration may be from any of these minimum durations to any of these maximum durations, for example from 1 to 18 months.

It will be understood, that the specific dose level for any particular patient will depend upon a variety of factors including the activity of the specific compound employed, the age, body weight, general health, sex, diet, time of administration, route of administration, and rate of excretion, drug combination i.e. other drugs being used to treat the patient), and the severity of the particular disorder undergoing therapy.

The terms “treatment” or “treating” of a subject includes the application or administration of a compound described herein to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, minimising or preventing the progression of cancer means treating the subject so as to prevent or delay the recurrence or metastasis of a tumor, or to prevent growth of an existing tumor. Minimising or preventing the progression of cancer includes preventing or delaying the recurrence of cancer, or preventing growth of an existing tumor, following treatment of cancer. The recurrence that is being prevented includes a recurrence for example, in the tumor bed, following surgical excision. Alternatively, recurrence includes metastasis of the cancer in another part of the body. The terms “preventing recurrence” and “preventing relapse” as used herein, are interchangeable.

The present invention also includes methods of preventing the development of cancer in an individual. For example, the individual for whom prevention of cancer is required may be considered to be at risk of developing cancer, but does not yet have detectable cancer. An individual at risk of the development of cancer may be an individual with a family history of cancer, and/or an individual for whom genetic testing or other testing indicates a high risk or high likelihood of the development of cancer. The individual may have cancer stem cells but does not yet have any detectable tumors. It will be understood that methods of preventing the development of cancer include methods of delaying the onset of cancer in a subject.

The terms “subject” and “patient” will be understood to be interchangeable. Although the invention finds application in humans, the invention is also useful for therapeutic veterinary purposes. The invention is useful for domestic or farm animals such as cattle, sheep, horses and poultry; for companion animals such as cats and dogs; and for zoo animals.

### Kits

There is also provided a kit for use according to the present invention comprising one or more of the following:
(i) an agent for use according to the invention or expression construct(s) encoding same.

In some embodiments, the kit may further comprise the immune checkpoint inhibitor optionally formulated into a pharmaceutical composition with one or more pharmaceutically acceptable excipients, carriers and/or diluents.

In the case of a kit for detecting cancer, the kit can additionally comprise a detection means, e.g., linked to an antigen binding protein for use according to the invention.

In the case of a kit for therapeutic/prophylactic use, the kit can additionally comprise a pharmaceutically acceptable carrier.

Optionally a kit of the invention is packaged with instructions for use in a method described herein according to any example.

TABLE 1
Summary of amino acid and nucleotide sequences for CAIX-binding
antibodies

| | | SEQ ID | |
|---|---|---|---|
| Antibody ID | Region | NO: | Amino acid or nucleotide sequence |
| Girentuximab | HCDR1 (protein) | 1 | NYYMS |
| chimeric VHO | HCDR2 (protein) | 2 | AINSDGGITYYLDTVKG |
| Variable Heavy | HCDR3 (protein) | 3 | HRSGYFSMDY |
| chain | VH (protein) | 4 | DVKLVESGGGLVKLGGSLKLSCAASGFTFSNYYMSWV |
| | | | RQTPEKRLELVAAINSDGGITYYLDTVKGRFTISRDNAK |
| | | | NTLYLQMSSLKSEDTALFYCARHRSGYFSMDYWGQG |
| | | | TSVTVSS |
| | HCDR1 (DNA) | 5 | AACTATTACATGTCT |
| | HCDR2 (DNA) | 6 | GCCATTAATAGTGATGGTGGTATCACCTACTATCTAG |
| | | | ACACTGTGAAGGGC |
| | HCDR3 (DNA) | 7 | CACCGCTCGGGCTACTTTTCTATGGACTAC |
| | VH (DNA) | 8 | GACGTGAAGCTCGTGGAGTCTGGGGGAGGCTTAGT |
| | | | GAAGCTTGGAGGATCCCTGAAACTCTCCTGTGCAGC |
| | | | CTCTGGATTCACTTTCAGTAACTATTACATGTCTTGG |
| | | | GTTCGCCAGACTCCAGAGAAGAGGCTGGAGTTGGT |
| | | | CGCAGCCATTAATAGTGATGGTGGTATCACCTACTA |
| | | | TCTAGACACTGTGAAGGGCCGATTCACCATTTCAAG |
| | | | AGACAATGCCAAGAACACCCTGTACCTGCAAATGAG |
| | | | CAGTCTGAAGTCTGAGGACACAGCCTTGTTTTACTG |
| | | | TGCAAGACACCGCTCGGGCTACTTTTCTATGGACTA |
| | | | CTGGGGTCAAGGAACCTCAGTCACCGTCTCCTCA |
| | HFR1 (protein) | 9 | DVKLVESGGGLVKLGGSLKLSCAASGFTFS |
| | HFR2 (protein) | 10 | WRQTPEKRLELVA |
| | HFR3 (protein) | 11 | RFTISRDNAKNTLYLQMSSLKSEDTALFYCAR |
| | HFR4 (protein) | 12 | WGQGTSVTVSS |
| | HFR1 (DNA) | 13 | GACGTGAAGCTCGTGGAGTCTGGGGGAGGCTTAGT |
| | | | GAAGCTTGGAGGATCCCTGAAACTCTCCTGTGCAGC |
| | | | CTCTGGATTCACTTTCAGT |
| | HFR2 (DNA) | 14 | TGGGTTCGCCAGACTCCAGAGAAGAGGCTGGAGTT |
| | | | GGTCGCA |
| | HFR3 (DNA) | 15 | CGATTCACCATTTCAAGAGACAATGCCAAGAACACC |
| | | | CTGTACCTGCAAATGAGCAGTCTGAAGTCTGAGGAC |
| | | | ACAGCCTTGTTTTACTGTGCAAGA |
| | HFR4 (DNA) | 16 | TGGGGTCAAGGAACCTCAGTCACCGTCTCCTCA |
| Girentuximab | HCDR1 (protein) | 17 | NYYMS |
| humanised VH1 | HCDR2 (protein) | 18 | AINSDGGITYYLDTVKG |
| Variable Heavy | HCDR3 (protein) | 19 | HRSGYFSMDY |
| chain | VH (protein) | 20 | DVKLVESGGGLVKPGGSLRLSCAASGFTFSNYYMSW |
| | | | VRQAPGKGLELVAAINSDGGITYYLDTVKGRFTISRDN |
| | | | AKNTLYLQMSSLKSEDTALYYCARHRSGYFSMDYWG |
| | | | QGTSVTVSS |
| | HCDR1 (DNA) | 21 | AACTACTACATGAGC |
| | HCDR2 (DNA) | 22 | GCCATTAACAGTGACGGTGGCATCACCTACTACCTG |
| | | | GACACCGTGAAGGGC |
| | HCDR3 (DNA) | 23 | CACAGGAGCGGCTACTTCTCTATGGACTAC |
| | VH (DNA) | 24 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAGCAGCCTGAAGAGCGAGGACACGGCCCTGTATT |
| | | | ACTGTGCGAGACACAGGAGCGGCTACTTCTCTATGG |
| | | | ACTACTGGGGCCAGGGCACCAGCGTCACTGTCTCC |
| | | | TCA |
| | HFR1 (protein) | 25 | DVKLVESGGGLVKPGGSLRLSCAASGFTFS |
| | HFR2 (protein) | 26 | WVRQAPGKGLELVA |
| | HFR3 (protein) | 27 | RFTISRDNAKNTLYLQMSSLKSEDTALYYCAR |
| | HFR4 (protein) | 28 | WGQGTSVTVSS |
| | HFR1 (DNA) | 29 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 30 | TGGGTGCGCCAGGCTCCAGGGAAGGGGCTGGAGC |
| | | | TGGTTGCC |
| | HFR3 (DNA) | 31 | CGATTCACCATCTCCAGGGACAACGCCAAGAACACC |
| | | | CTGTATCTGCAAATGAGCAGCCTGAAGAGCGAGGA |
| | | | CACGGCCCTGTATTACTGTGCGAGA |
| | HFR4 (DNA) | 32 | TGGGGCCAGGGCACCAGCGTCACTGTCTCCTCA |
| Girentuximab | HCDR1 (protein) | 33 | NYYMS |
| humanised VH3 | HCDR2 (protein) | 34 | AINSDGGITYYLDTVKG |
| Variable Heavy | HCDR3 (protein) | 35 | HRSGYFSMDY |
| chain | VH (protein) | 36 | DVKLVESGGGLVKPGGSLRLSCAASGFTFSNYYMSW |
| | | | VRQAPGKGLELVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 37 | AACTACTACATGAGC |
| | HCDR2 (DNA) | 38 | CCATTAACAGTGACGGTGGCATCACCTACTACCTGG |
| | | | ACACCGTGAAGGGC |
| | HCDR3 (DNA) | 39 | CACAGGAGCGGCTACTTCTCTATGGACTAC |
| | VH (DNA) | 40 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 41 | DVKLVESGGGLVKPGGSLRLSCAASGFTFS |
| | HFR2 (protein) | 42 | WVRQAPGKGLELVA |
| | HFR3 (protein) | 43 | RFTISRDNAKNTLYLQMNSLRAEDTALYYCAR |
| | HFR4 (protein) | 44 | WGQGTLVTVSS |
| | HFR1 (DNA) | 45 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 46 | TGGGTGCGCCAGGCTCCAGGGAAGGGGCTGGAGC |
| | | | TGGTTGCC |
| | HFR3 (DNA) | 47 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 48 | TGGGGCCAGGGCACCCTGGTCACTGTCTCCTCA |
| Girentuximab | HCDR1 (protein) | 49 | NYYMS |
| humanised VH4 | HCDR2 (protein) | 50 | AINSDGGITYYLDTVKG |
| Variable Heavy | HCDR3 (protein) | 51 | HRSGYFSMDY |
| chain | VH (protein) | 52 | EVQLVESGGGLVKPGGSLRLSCAASGFTFSNYYMSW |
| | | | VRQAPGKGLELVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 53 | AACTACTACATGAGC |
| | HCDR2 (DNA) | 54 | GCCATTAACAGTGACGGTGGCATCACCTACTACCTG |
| | | | GACACCGTGAAGGGC |
| | HCDR3 (DNA) | 55 | CACAGGAGCGGCTACTTCTCTATGGACTAC |
| | VH (DNA) | 56 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 57 | EVQLVESGGGLVKPGGSLRLSCAASGFTFS |
| | HFR2 (protein) | 58 | WVRQAPGKGLELVA |
| | HFR3 (protein) | 59 | RFTISRDNAKNTLYLQMNSLRAEDTALYYCAR |
| | HFR4 (protein) | 60 | WGQGTLVTVSS |
| | HFR1 (DNA) | 61 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 62 | TGGGTGCGCCAGGCTCCAGGGAAGGGGCTGGAGC |
| | | | TGGTTGCC |
| | HFR3 (DNA) | 63 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 64 | TGGGGCCAGGGCACCCTGGTCACTGTCTCCTCA |
| Girentuximab | HCDR1 (protein) | 65 | NYYMS |
| humanised VH5 | HCDR2 (protein) | 66 | AINSDGGITYYLDTVKG |
| Variable Heavy | HCDR3 (protein) | 67 | HRSGYFSMDY |
| chain | VH (protein) | 68 | EVQLVESGGGLVKPGGSLRLSCAASGFTFSNYYMSW |
| | | | VRQAPGKGLEWVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 69 | AACTACTACATGAGC |
| | HCDR2 (DNA) | 70 | GCCATTAACAGTGACGGTGGCATCACCTACTACCTG |
| | | | GACACCGTGAAGGGC |
| | HCDR3 (DNA) | 71 | CACAGGAGCGGCTACTTCTCTATGGACTAC |
| | VH (DNA) | 72 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGTG |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 73 | EVQLVESGGGLVKPGGSLRLSCAASGFTFS |
| | HFR2 (protein) | 74 | WVRQAPGKGLEWVA |
| | HFR3 (protein) | 75 | RFTISRDNAKNTLYLQMNSLRAEDTALYYCAR |
| | HFR4 (protein) | 76 | WGQGTLVTVSS |
| | HFR1 (DNA) | 77 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 78 | TGGGTGCGCCAGGCTCCAGGGAAGGGGCTGGAGT |
| | | | GGGTTGCC |
| | HFR3 (DNA) | 79 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 80 | TGGGGCCAGGGCACCCTGGTCACTGTCTCCTCA |
| Girentuximab | LCDR1 (protein) | 81 | KASQNVVSAVA |
| Chimeric Vk0 | | | |
| Variable light | LCDR2 | 82 | SASNRYT |
| chain | (protein) | | |
| | LCDR3 (protein) | 83 | QQYSNYPWT |
| | VL (protein) | 84 | DIVMTQSQRFMSTTVGDRVSITCKASQNVVSAVAWYQ |
| | | | QKPGQSPKLLIYSASNRYTGVPDRFTGSGSGTDFTLTI |
| | | | SNMQSEDLADFFCQQYSNYPWTFGGGTKLEIK |
| | LCDR1 (DNA) | 85 | AAGGCCAGTCAGAATGTGGTTTCTGCTGTTGCC |
| | LCDR2 (DNA) | 86 | TCAGCATCCAATCGGTACACT |
| | LCDR3 (DNA) | 87 | CAACAATATAGCAACTATCCGTGGACG |
| | VL (DNA) | 88 | GACATTGTGATGACCCAGTCTCAAAGATTCATGTCC |
| | | | ACAACAGTAGGAGACAGGGTCAGCATCACCTGCAA |
| | | | GGCCAGTCAGAATGTGGTTTCTGCTGTTGCCTGGTA |
| | | | TCAACAGAAACCAGGACAATCTCCTAAACTACTGATT |
| | | | TACTCAGCATCCAATCGGTACACTGGAGTCCCTGAT |
| | | | CGCTTCACAGGCAGTGGATCTGGGACAGATTTCACT |
| | | | CTCACCATTAGCAATATGCAGTCTGAAGACCTGGCT |
| | | | GATTTTTTCTGTCAACAATATAGCAACTATCCGTGGA |
| | | | CGTTCGGTGGAGGCACCAAGCTGGAAATCAAA |
| | LFR1 | 89 | DIVMTQSQRFMSTTVGDRVSITC |
| | (protein) | | |
| | LFR2 (protein) | 90 | WYQQKPGQSPKLLIY |
| | LFR3 (protein) | 91 | GVPDRFTGSGSGTDFTLTISNMQSEDLADFFC |
| | LFR4 | 92 | FGGGTKLEIK |
| | (protein) | | |
| | LFR1 | 93 | GACATTGTGATGACCCAGTCTCAAAGATTCATGTCC |
| | (DNA) | | ACAACAGTAGGAGACAGGGTCAGCATCACCTGC |
| | LFR2 (DNA) | 94 | TGGTATCAACAGAAACCAGGACAATCTCCTAAACTA |
| | | | CTGATTTAC |
| | LFR3 (DNA) | 95 | GGAGTCCCTGATCGCTTCACAGGCAGTGGATCTGG |
| | | | GACAGATTTCACTCTCACCATTAGCAATATGCAGTCT |
| | | | GAAGACCTGGCTGATTTTTTCTGT |
| | LFR4 | 96 | TTCGGTGGAGGCACCAAGCTGGAAATCAAA |
| | (DNA) | | |
| Girentuximab | LCDR1 (protein) | 97 | KASQNVVSAVA |
| Humanised Vk1 | LCDR2 | 98 | SASNRYT |
| Variable light | (protein) | | |
| chain | LCDR3 (protein) | 99 | QQYSNYPWT |
| | VL (protein) | 100 | DIVMTQSPSFLSASVGDRVTITCKASQNVVSAVAWYQ |
| | | | QKPGQAPKLLIYSASNRYTGVPDRFTGSGSGTDFTLTI |
| | | | SSLQSEDLADYFCQQYSNYPWTFGGGTKVEIK |
| | LCDR1 (DNA) | 101 | AAGGCAAGTCAGAACGTGGTGAGTGCTGTGGCC |
| | LCDR2 (DNA) | 102 | AGCGCCTCCAACAGGTACACC |
| | LCDR3 (DNA) | 103 | CAACAGTACAGCAATTACCCTTGGACG |
| | VL (DNA) | 104 | GACATCGTGATGACCCAGTCTCCATCCTTCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAAGCTCCTGAT |
| | | | CTATAGCGCCTCCAACAGGTACACCGGGGTCCCAG |
| | | | ACAGGTTCACCGGCAGTGGATCTGGGACAGATTTCA |
| | | | CTCTCACCATCAGCAGCCTGCAGAGCGAAGATCTG |
| | | | GCAGACTATTTCTGTCAACAGTACAGCAATTACCCTT |
| | | | GGACGTTCGGCGGCGGGACCAAGGTGGAAATCAAA |
| | LFR1 | 105 | DIVMTQSPSFLSASVGDRVTITC |
| | (protein) | | |
| | LFR2 (protein) | 106 | WYQQKPGQAPKLLIY |
| | LFR3 (protein) | 107 | GVPDRFTGSGSGTDFTLTISSLQSEDLADYFC |
| | LFR4 | 108 | FGGGTKVEIK |
| | (protein) | | |
| | LFR1 | 109 | GACATCGTGATGACCCAGTCTCCATCCTTCCTGTCT |
| | (DNA) | | GCATCTGTAGGAGACAGAGTCACCATCACTTGC |
| | LFR2 (DNA) | 110 | TGGTATCAGCAGAAACCAGGGCAGGCTCCTAAGCT |
| | | | CCTGATCTAT |
| | LFR3 (DNA) | 111 | GGGGTCCCAGACAGGTTCACCGGCAGTGGATCTGG |
| | | | GACAGATTTCACTCTCACCATCAGCAGCCTGCAGAG |
| | | | CGAAGATCTGGCAGACTATTTCTGT |
| | LFR4 | 112 | TTCGGCGGCGGGACCAAGGTGGAAATCAAA |
| | (DNA) | | |
| Girentuximab | LCDR1 (protein) | 113 | KASQNVVSAVA |
| Humanised Vk2 | LCDR2 | 114 | SASNRYT |
| Variable light | (protein) | | |
| chain | LCDR3 (protein) | 115 | QQYSNYPWT |
| | VL (protein) | 116 | DIVMTQSPSSLSASVGDRVTITCKASQNVVSAVAWYQ |
| | | | QKPGQAPRLLIYSASNRYTGVPDRFTGSGSGTDFTLTI |
| | | | SSLQAEDLADYFCQQYSNYPWTFGGGTKVEIK |
| | LCDR1 (DNA) | 117 | AAGGCAAGTCAGAACGTGGTGAGTGCTGTGGCC |
| | LCDR2 (DNA) | 118 | AGCGCCTCCAACAGGTACACC |
| | LCDR3 (DNA) | 119 | CAACAGTACAGCAATTACCCTTGGACG |
| | VL (DNA) | 120 | GACATCGTGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCACCGGCAGTGGATCTGGGACAGATTT |
| | | | CACTCTCACCATCAGCAGCCTGCAGGCCGAAGATCT |
| | | | GGCAGACTATTTCTGTCAACAGTACAGCAATTACCC |
| | | | TTGGACGTTCGGCGGCGGGACCAAGGTGGAAATCA |
| | | | AA |
| | LFR1 | 121 | DIVMTQSPSSLSASVGDRVTITC |
| | (protein) | | |
| | LFR2 (protein) | 122 | WYQQKPGQAPRLLIY |
| | LFR3 (protein) | 123 | GVPDRFTGSGSGTDFTLTISSLQAEDLADYFC |
| | LFR4 | 124 | FGGGTKVEIK |
| | (protein) | | |
| | LFR1 | 125 | GACATCGTGATGACCCAGTCTCCATCCAGCCTGTCT |
| | (DNA) | | GCATCTGTAGGAGACAGAGTCACCATCACTTGC |
| | LFR2 (DNA) | 126 | TGGTATCAGCAGAAACCAGGGCAGGCTCCTAGGCT |
| | | | CCTGATCTAT |
| | LFR3 (DNA) | 127 | GGGGTCCCAGACAGGTTCACCGGCAGTGGATCTGG |
| | | | GACAGATTTCACTCTCACCATCAGCAGCCTGCAGGC |
| | | | CGAAGATCTGGCAGACTATTTCTGT |
| | LFR4 | 128 | TTCGGCGGCGGGACCAAGGTGGAAATCAAA |
| | (DNA) | | |
| Girentuximab | LCDR1 (protein) | 129 | KASQNVVSAVA |
| Humanised Vk3 | LCDR2 | 130 | SASNRYT |
| Variable light | (protein) | | |
| chain | LCDR3 (protein) | 131 | QQYSNYPWT |
| | VL (protein) | 132 | DIQMTQSPSSLSASVGDRVTITCKASQNVVSAVAWYQ |
| | | | QKPGQAPRLLIYSASNRYTGVPDRFSGSGSGTDFTLTI |
| | | | SSLQAEDLADYFCQQYSNYPWTFGGGTKVEIK |
| | LCDR1 (DNA) | 133 | AAGGCAAGTCAGAACGTGGTGAGTGCTGTGGCC |
| | LCDR2 (DNA) | 13 | AGCGCCTCCAACAGGTACACC |
| | LCDR3 (DNA) | 135 | CAACAGTACAGCAATTACCCTTGGACG |
| | VL (DNA) | 136 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTTCTGTCAACAGTACAGCAATTACCC |
| | | | TTGGACGTTCGGCGGCGGGACCAAGGTGGAAATCA |
| | | | AA |
| | LFR1 | 137 | DIQMTQSPSSLSASVGDRVTITC |
| | (protein) | | |
| | LFR2 (protein) | 138 | WYQQKPGQAPRLLIY |
| | LFR3 (protein) | 139 | GVPDRFSGSGSGTDFTLTISSLQAEDLADYFC |
| | LFR4 | 140 | FGGGTKVEIK |
| | (protein) | | |
| | LFR1 | 141 | GACATCCAGATGACCCAGTCTCCATCCAGCCTGTCT |
| | (DNA) | | GCATCTGTAGGAGACAGAGTCACCATCACTTGC |
| | LFR2 (DNA) | 142 | TGGTATCAGCAGAAACCAGGGCAGGCTCCTAGGCT |
| | | | CCTGATCTAT |
| | LFR3 (DNA) | 143 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTTCTGT |
| | LFR4 | 144 | TTCGGCGGCGGGACCAAGGTGGAAATCAAA |
| | (DNA) | | |
| Girentuximab | LCDR1 (protein) | 145 | KASQNVVSAVA |
| Humanised Vk4 | LCDR2 | 146 | SASNRYT |
| Variable light | (protein) | | |
| chain | LCDR3 (protein) | 147 | QQYSNYPWT |
| | VL (protein) | 148 | DIQMTQSPSSLSASVGDRVTITCKASQNVVSAVAWYQ |
| | | | QKPGQAPRLLIYSASNRYTGVPDRFSGSGSGTDFTLTI |
| | | | SSLQAEDLADYYCQQYSNYPWTFGGGTKVEIK |
| | LCDR1 (DNA) | 149 | AAGGCAAGTCAGAACGTGGTGAGTGCTGTGGCC |
| | LCDR2 (DNA) | 150 | AGCGCCTCCAACAGGTACACC |
| | LCDR3 (DNA) | 151 | CAACAGTACAGCAATTACCCTTGGACG |
| | VL (DNA) | 152 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTACTGTCAACAGTACAGCAATTACCC |
| | | | TTGGACGTTCGGCGGCGGGACCAAGGTGGAAATCA |
| | | | AA |
| | LFR1 | 153 | DIQMTQSPSSLSASVGDRVTITC |
| | (protein) | | |
| | LFR2 (protein) | 154 | WYQQKPGQAPRLLIY |
| | LFR3 (protein) | 155 | GVPDRFSGSGSGTDFTLTISSLQAEDLADYYC |
| | LFR4 | 156 | FGGGTKVEIK |
| | (protein) | | |
| | LFR1 | 157 | GACATCCAGATGACCCAGTCTCCATCCAGCCTGTCT |
| | (DNA) | | GCATCTGTAGGAGACAGAGTCACCATCACTTGC |
| | LFR2 (DNA) | 158 | TGGTATCAGCAGAAACCAGGGCAGGCTCCTAGGCT |
| | | | CCTGATCTAT |
| | LFR3 (DNA) | 159 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTACTGT |
| | LFR4 | 160 | TTCGGCGGCGGGACCAAGGTGGAAATCAAA |
| | (DNA) | | |
| Girentuximab | LCDR1 (protein) | 161 | KASQNVVSAVA |
| Humanised Vk5 | LCDR2 | 162 | SASNRYT |
| Variable light | (protein) | | |
| chain | LCDR3 (protein) | 163 | QQYSNYPWT |
| | VL (protein) | 164 | DIQMTQSPSSLSASVGDRVTITCKASQNVVSAVAWYQ |
| | | | QKPGQAPRRLIYSASNRYTGVPDRFSGSGSGTDFTLTI |
| | | | SSLQAEDLADYYCQQYSNYPWTFGGGTKVEIK |
| | LCDR1 (DNA) | 165 | AAGGCAAGTCAGAACGTGGTGAGTGCTGTGGCC |
| | LCDR2 (DNA) | 166 | AGCGCCTCCAACAGGTACACC |
| | LCDR3 (DNA) | 167 | CAACAGTACAGCAATTACCCTTGGACG |
| | VL (DNA) | 168 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGAGGCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTACTGTCAACAGTACAGCAATTACCC |
| | | | TTGGACGTTCGGCGGCGGGACCAAGGTGGAAATCA |
| | | | AA |
| | LFR1 | 169 | DIQMTQSPSSLSASVGDRVTITC |
| | (protein) | | |
| | LFR2 (protein) | 170 | WYQQKPGQAPRRLIY |
| | LFR3 (protein) | 171 | GVPDRFSGSGSGTDFTLTISSLQAEDLADYYC |
| | LFR4 | 172 | FGGGTKVEIK |
| | (protein) | | |
| | LFR1 | 173 | GACATCCAGATGACCCAGTCTCCATCCAGCCTGTCT |
| | (DNA) | | GCATCTGTAGGAGACAGAGTCACCATCACTTGC |
| | LFR2 (DNA) | 174 | TGGTATCAGCAGAAACCAGGGCAGGCTCCTAGGAG |
| | | | GCTGATCTAT |
| | LFR3 (DNA) | 175 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTACTGT |
| | LFR4 | 176 | TTCGGCGGCGGGACCAAGGTGGAAATCAAA |
| | (DNA) | | |
| Girentuximab | IgG1 HC | 177 | ASTKGPSVFPLAPSSKSTSGGTAALGCLVKDYFPEPVT |
| unmodified | (protein) | | VSWNSGALTSGVHTFPAVLQSSGLYSLSSVVTVPSSS |
| human IgG1 | | | LGTQTYICNVNHKPSNTKVDKKVEPKSCDKTHTCPPC |
| constant chain | | | PAPELLGGPSVFLFPPKPKDTLMISRTPEVTCVVVDVS |
| region | | | HEDPEVKFNWYVDGVEVHNAKTKPREEQYNSTYRVV |
| | | | SVLTVLHQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSR |
| | | | WQQGNVFSCSVMHEALHNHYTQKSLSLSPGK |
| Girentuximab | IgG1 H310A | 178 | ASTKGPSVFPLAPSSKSTSGGTAALGCLVKDYFPEPVT |
| modified human | H435Q HC | | VSWNSGALTSGVHTFPAVLQSSGLYSLSSVVTVPSSS |
| IgG1 constant | (protein) | | LGTQTYICNVNHKPSNTKVDKKVEPKSCDKTHTCPPC |
| chain region | | | PAPELLGGPSVFLFPPKPKDTLMISRTPEVTCVVVDVS |
| | | | HEDPEVKFNWYVDGVEVHNAKTKPREEQYNSTYRVV |
| | | | SVLTVLAQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSR |
| | | | WQQGNVFSCSVMHEALHNQYTQKSLSLSPGK |
| Girentuximab | IgG4 S228P | 179 | ASTKGPSVFPLAPCSRSTSESTAALGCLVKDYFPEPVT |
| modified human | L235E HC | | VSWNSGALTSGVHTFPAVLQSSGLYSLSSVVTVPSSS |
| IgG4 constant | (protein) | | LGTKTYTCNVDHKPSNTKVDKRVESKYGPPCPPCPAP |
| chain region | | | EFEGGPSVFLFPPKPKDTLMISRTPEVTCVVVDVSQED |
| | | | PEVQFNWYVDGVEVHNAKTKPREEQFNSTYRVVSVLT |
| | | | VLHQDWLNGKEYKCKVSNKGLPSSIEKTISKAKGQPR |
| | | | EPQVYTLPPSQEEMTKNQVSLTCLVKGFYPSDIAVEW |
| | | | ESNGQPENNYKTTPPVLDSDGSFFLYSRLTVDKSRWQ |
| | | | EGNVFSCSVMHEALHNHYTQKSLSLSLGK |
| Girentuximab | IgG4 S228P | 180 | ASTKGPSVFPLAPCSRSTSESTAALGCLVKDYFPEPVT |
| modified human | L235E H310A | | VSWNSGALTSGVHTFPAVLQSSGLYSLSSVVTVPSSS |
| IgG4 constant | H435Q HC | | LGTKTYTCNVDHKPSNTKVDKRVESKYGPPCPPCPAP |
| chain region | (protein) | | EFEGGPSVFLFPPKPKDTLMISRTPEVTCVVVDVSQED |
| | | | PEVQFNWYVDGVEVHNAKTKPREEQFNSTYRVVSVLT |
| | | | VLAQDWLNGKEYKCKVSNKGLPSSIEKTISKAKGQPRE |
| | | | PQVYTLPPSQEEMTKNQVSLTCLVKGFYPSDIAVEWE |
| | | | SNGQPENNYKTTPPVLDSDGSFFLYSRLTVDKSRWQE |
| | | | GNVFSCSVMHEALHNQYTQKSLSLSLGK |
| Girentuximab | Human κ LC | 181 | RTVAAPSVFIFPPSDEQLKSGTASVVCLLNNFYPREAK |
| kappa light chain | constant region | | VQWKVDNALQSGNSQESVTEQDSKDSTYSLSSTLTLS |
| constant region | | | KADYEKHKVYACEVTHQGLSSPVTKSFNRGEC |
| Girentuximab | IgG1 VH4 heavy | 182 | EVQLVESGGGLVKPGGSLRLSCAASGFTFSNYYMSW |
| native IgG1 | chain | | VRQAPGKGLELVAAINSDGGITYYLDTVKGRFTISRDN |
| allotype Gm | | | AKNTLYLQMNSLRAEDTALYYCARHRSGYFSMDYWG |
| (1,17) VH4 | | | QGTLVTVSSASTKGPSVFPLAPSSKSTSGGTAALGCLV |
| heavy chain | | | KDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLHQDWLNGKEYKCKVSNKALPAPIE |
| | | | KTISKAKGQPREPQVYTLPPSRDELTKNQVSLTCLVKG |
| | | | FYPSDIAVEWESNGQPENNYKTTPPVLDSDGSFFLYS |
| | | | KLTVDKSRWQQGNVFSCSVMHEALHNHYTQKSLSLS |
| | | | PGK |
| hGirentuximab | FcRn-null IgG1 | 183 | EVQLVESGGGLVKPGGSLRLSCAASGFTFSNYYMSW |
| IgG1 allotype | allotype H130A | | VRQAPGKGLELVAAINSDGGITYYLDTVKGRFTISRDN |
| Gm (1,17) | H435Q | | AKNTLYLQMNSLRAEDTALYYCARHRSGYFSMDYWG |
| RADmAb VH4 | | | QGTLVTVSSASTKGPSVFPLAPSSKSTSGGTAALGCLV |
| heavy chain | | | KDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLAQDWLNGKEYKCKVSNKALPAPIE |
| | | | KTISKAKGQPREPQVYTLPPSRDELTKNQVSLTCLVKG |
| | | | FYPSDIAVEWESNGQPENNYKTTPPVLDSDGSFFLYS |
| | | | KLTVDKSRWQQGNVFSCSVMHEALHNQYTQKSLSLS |
| | | | PGK |
| hGirentuximab | FcRγ-null IgG4 | 184 | EVQLVESGGGLVKPGGSLRLSCAASGFTFSNYYMSW |
| IgG4 RADmAb | S228P L235E | | VRQAPGKGLELVAAINSDGGITYYLDTVKGRFTISRDN |
| VH4 heavy chain | | | AKNTLYLQMNSLRAEDTALYYCARHRSGYFSMDYWG |
| | | | QGTLVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFEGGPSVFLFPPKPKDTLMISRTPEVTC |
| | | | VVVDVSQEDPEVQFNWYVDGVEVHNAKTKPREEQFN |
| | | | STYRVVSVLTVLHQDWLNGKEYKCKVSNKGLPSSIEKT |
| | | | ISKAKGQPREPQVYTLPPSQEEMTKNQVSLTCLVKGF |
| | | | YPSDIAVEWESNGQPENNYKTTPPVLDSDGSFFLYSR |
| | | | LTVDKSRWQEGNVFSCSVMHEALHNHYTQKSLSLSLG |
| | | | K |
| hGirentuximab | FcRn + FcRγ-null | 185 | EVQLVESGGGLVKPGGSLRLSCAASGFTFSNYYMSW |
| IgG4 RADmAb | IgG4 S228P | | VRQAPGKGLELVAAINSDGGITYYLDTVKGRFTISRDN |
| VH4 heavy chain | L235E H310A | | AKNTLYLQMNSLRAEDTALYYCARHRSGYFSMDYWG |
| | H435Q | | QGTLVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFEGGPSVFLFPPKPKDTLMISRTPEVTC |
| | | | VVVDVSQEDPEVQFNWYVDGVEVHNAKTKPREEQFN |
| | | | STYRVVSVLTVLAQDWLNGKEYKCKVSNKGLPSSIEKT |
| | | | ISKAKGQPREPQVYTLPPSQEEMTKNQVSLTCLVKGF |
| | | | YPSDIAVEWESNGQPENNYKTTPPVLDSDGSFFLYSR |
| | | | LTVDKSRWQEGNVFSCSVMHEALHNQYTQKSLSLSL |
| | | | GK |
| hGirentuximab | Vκ Light Chain | 186 | DIQMTQSPSSLSASVGDRVTITCKASQNVVSAVAWYQ |
| Vκ Light Chain | | | QKPGQAPRLLIYSASNRYTGVPDRFSGSGSGTDFTLTI |
| | | | SSLQAEDLADYYCQQYSNYPWTFGGGTKVEIKRTVAA |
| | | | PSVFIFPPSDEQLKSGTASVVCLLNNFYPREAKVQWKV |
| | | | DNALQSGNSQESVTEQDSKDSTYSLSSTLTLSKADYE |
| | | | KHKVYACEVTHQGLSSPVTKSFNRGEC |

### EXAMPLES

### Example 1: Antibodies for Binding to CAIX

### Summary

Humanised antibody variable region genes were cloned into vectors encoding an unmodified human IgG1 heavy chain constant domain and human kappa light chain constant domain. The chimeric antibody was additionally cloned into vectors encoding either a modified human IgG1 heavy chain constant domain containing the mutations H310A and H435Q that abolish FcRn and Protein A binding (Andersen et al, 2012) or a modified human IgG4 heavy chain construct containing the mutations S228P (to stabilise the hinge (Angal et al, 1993)) and L235E (to remove effector function (Reddy et al, 2000)) as well as the FcRn abolishing mutations described above. Chimeric and humanised antibodies were transiently expressed in HEK EBNA cells and tested for binding to human carbonic anhydrase IX (CAIX) using Biacore single cycle kinetic analysis. Selected lead antibodies were purified by Protein A or Protein G and analysed by analytical SEC, competition ELISA and Biacore multicycle kinetic analysis.

Three lead candidate humanised antibodies with similar binding to the chimeric antibody were identified. These were subsequently cloned into vectors encoding the human IgG1 heavy chain constant domain (H310A, H435Q) and IgG4 heavy chain constant domain (S241P, L235E, H310A, H435Q) as described above. Chimeric and lead humanised antibodies were transiently expressed in CHO cells and tested for binding to human carbonic anhydrase IX (CAIX) using Biacore single cycle kinetic analysis. Antibodies were purified by either Protein A or Protein G and analysed by analytical SEC and Biacore multicycle kinetic analysis.

### Methods

### Design of Composite Human Antibody™ Variable Region Sequences

Structural models of the Girentuximab antibody V regions were produced using Swiss PDB and analysed in order to identify important “constraining” amino acids that were likely to be essential for the binding properties of the antibody. Most residues contained within the CDRs (using both Kabat and Chothia definitions) together with a number of framework residues were considered to be important. The VH and Vκ sequences of Girentuximab contain typical framework residues and CDR 1, 2 and 3 motifs that are comparable to many murine antibodies.

From the above analysis, it was considered that Composite Human sequences of Girentuximab could be created with a wide latitude for alternative residues outside of the CDRs but with only a narrow menu of possible residues within the CDR sequences. Preliminary analysis indicated that corresponding sequence segments from several human antibodies could be combined to create CDRs similar or identical to those in the murine sequences. For regions outside of, and flanking the CDRs, a wide selection of human sequence segments were identified as possible components of the novel humanised V regions.

### CD4+ T Cell Epitope Avoidance

Based upon the structural analysis, a large preliminary set of sequence segments were identified that could be used to create Girentuximab humanised variants. These segments were selected and analysed using iTope™ technology for in silico analysis of peptide binding to human MHC class II alleles (Perry et al, 2008), and using the TCED™ of known antibody sequence-related T cell epitopes (Bryson et al, 2010). Sequence segments that were identified as significant non-human germline binders to human MHC class II or that scored significant hits against the TCED™ were discarded. This resulted in a reduced set of segments, and combinations of these were again analysed, as above, to ensure that the junctions between segments did not contain potential T cell epitopes. Selected sequence segments were assembled into complete V region sequences that were devoid of significant T cell epitopes. Five heavy chain (VH0 (Chimeric), VH1, VH3, VH4 and VH5) and six light chain (Vκ0 (Chimeric), Vκ1 to Vκ5) sequences were then chosen for gene synthesis and expression in mammalian cells (see Table 1).

### Construction of Chimeric Antibodies and Humanised Variants

The chimeric VH0 and Vκ0 Girentuximab sequences and its humanised variants were synthesised with flanking restriction enzyme sites for cloning into Abzena's pANT expression vector system for unmodified human IgG1 heavy chain (allotype G1m 1,17; equivalent to the Girentuximab allotype) (pANT68) and kappa light chain (pANT13.2). The VH regions were cloned between the Mlu I and Hind Ill restriction sites, and the Vκ regions were cloned between the BssH II and BamH I restriction sites. All constructs were confirmed by sequencing.

The FcRn abolishing mutations, H310A and H435Q, were incorporated by site directed mutagenesis into expression vectors for either human IgG1 heavy chain (equivalent allotype to Girentuximab) (pANT69) or for human IgG4 (S228P, L235E) heavy chain (pANT70). The VH0 was cloned into these two vectors in addition to pANT36.2 which encodes IgG4 (S228P, L235E).

### Expression of Antibodies in HEK293 EBNA Cells

Chimeric Girentuximab (VH0/Vκ0), two control antibodies (VH0/Vκ1, VH1/Vκ0) and combinations of VH and Vκ chains (a total of 20 pairings) were transiently expressed as IgG1 in HEK EBNA cells (LGC Standards, Teddington, UK) using a PEI transfection method. Cells were incubated for seven days post-transfection. Additionally, HEK EBNA cells were similarly transfected with chimeric Girentuximab VH0 and Vκ0 in an IgG1 (H310A, H435Q) heavy chain vector, an IgG4 (S228P, L235E) heavy chain vector and an IgG4 (S228P, L235E H310A, H435Q) heavy chain vector. Supernatant antibody titres were determined by ELISA.

### Kinetic Analysis of Chimeric and Humanised Variant Binding to Carbonic Anhydrase IX

In order to assess the binding of all Girentuximab Composite Human Antibody™ variants, single cycle kinetic analysis was performed on supernatants from transiently transfected HEK EBNA cell cultures. Kinetic experiments were performed on a Biacore T200 (serial no. 1909913) running Biacore T200 Control software V2.0.1 and Evaluation software V3.0 (GE Healthcare, Uppsala, Sweden). All single cycle kinetic experiments were run at 25 °C with HBS-P+ running buffer (pH 7.4) (GE Healthcare, Little Chalfont, UK). For direct comparison, all antibodies were captured on a Protein G chip (GE Healthcare, Uppsala, Sweden).

Antibodies were diluted in running buffer to a final concentration of 0.5 μg/ml, based on concentrations assessed by ELISA titre. At the start of each cycle, antibodies were loaded onto Fc2, Fc3 and Fc4 of the Protein G chip (GE Healthcare, Little Chalfont, UK). IgGs were captured at a flow rate of 10 μl/min to give an immobilisation level (RL) of ~79 RU, the theoretical value to obtain an Rmax of ~50 RU. The surface was then allowed to stabilise. Single cycle kinetic data was obtained with Carbonic Anhydrase IX (CAIX, Stratech, Newmarket, UK) as the analyte at a flow rate of 30 μl/min to minimise any potential mass transport limitations. Multiple repeats with the reference chimeric antibody (VH0/Vκ0 IgG1) were performed to check the stability of the surface and analyte over the kinetic cycles. The signal from the reference channel Fc1 (no antibody) was subtracted from that of Fc2, Fc3 and Fc4 to correct for differences in non-specific binding to a reference surface. A four point, two-fold dilution range from 3.125 nM to 25 nM CAIX without regeneration between each concentration was used. The association phase for the four injections of increasing concentrations of CAIX was monitored for 200 seconds each time and a single dissociation phase was measured for 300 seconds following the last injection of CAIX. Regeneration of the Protein G surface was conducted using an injection of 10 mM glycine-HCL pH 1.5 followed by an injection of 10 mM glycine-HCL pH 1.5 containing 0.5% P20.

Variants were analysed with the reference chimeric (VH0/Vκ0 IgG1) used for the calculation of the relative KD. The signal from each antibody blank run was subtracted to correct for differences in surface stability. Single cycle kinetics data demonstrated that all humanised variants bound to CAIX within two-fold of the reference chimeric antibody, except those containing a VH5 and/or a Vκ5, as these variants displayed lower affinities to CAIX.

### Purification of Chimeric and Lead Humanised Antibodies

Six IgG1 lead antibodies, VH3/Vκ2, VH3/Vκ3, VH3/Vκ4, VH4/Vκ2, VH4/Vκ3 and VH4/Vκ4 were purified based on humanness and single cycle kinetics data. The chimeric IgG1 antibody, lead humanised IgG1 antibodies and chimeric IgG4 (S228P, L235E) antibody were purified from cell culture supernatants using Protein A sepharose columns (GE Healthcare, Little Chalfont, UK). The chimeric IgG1 (H310A, H435Q) and IgG4 (S228P, L325E, H310A, H435Q) antibodies were purified from cell culture supernatant using HiTrap™ Protein G HP columns (GE Healthcare, Little Chalfont, UK) as the H310A H435Q double mutation has been shown to adversely affect binding to some Protein A resins. All antibodies were buffer exchanged into 1×PBS pH 7.2 and quantified by OD280 nm using an extinction coefficient (Ec(0.1%)) based on the predicted amino acid sequence. Antibodies were analysed by reducing SDS-PAGE of 2 μg loaded antibody and bands corresponding to the profile of a typical antibody were observed.

### Multicycle Kinetics Analysis of Chimeric and Lead Antibodies

Multicycle kinetics analysis was performed on the purified chimeric antibodies and six lead IgG1 antibodies using a Biacore T200 (serial no. 1909913) instrument running Biacore T200 Evaluation Software V3.0.1 (Uppsala, Sweden) to establish an accurate affinity for carbonic anhydrase IX. The purified antibodies were diluted to a concentration of 1 μg/ml in HBS-P+. At the start of each cycle, each antibody was captured on the Protein G surface to give an RL of ~79 RU. Following capture, the surface was allowed to stabilise. Kinetic data was obtained using a flow rate of 30 μl/min to minimise any potential mass transfer effects. For the kinetics analysis, carbonic anhydrase IX (CAIX) was used. Multiple repeats of the blank (CAIX) and a repeat of a single concentration of the analyte were programmed into the kinetic run in order to check the stability of both the surface and analyte over the kinetic cycles. For kinetic analysis, a two-fold dilution range was selected from 50 nM to 0.078 nM CAIX. The association phase of CAIX was monitored for 280 seconds and the dissociation phase was monitored for 300 seconds. Regeneration of the Protein G surface was conducted using an injection of 10 mM glycine-HCL pH 1.5 followed by an injection of 10 mM glycine-HCL pH 1.5 containing 0.5% P20. The signal from the reference channel Fc1 was subtracted from that of Fc2, Fc3 and Fc4 to correct for differences in non-specific binding to a reference surface, and a global Rmax parameter was used in the 1-to-1 binding model. The relative KD was calculated by dividing the KD of the humanised variants by that of the chimeric antibody on the same chip. All the lead humanised IgG1 variants and the chimeric antibodies on different IgG backbones showed relative KDs within 2-fold of the chimeric IgG1 antibody.

### Carbonic Anhydrase IX Competition ELISA

Lead purified IgG1 variants and chimeric antibodies expressed with different IgG constant domains (IgG1, IgG1 (H310A, H435Q), IgG4 (S228P, L235E) and IgG4 (S228P, L235E, H310A, H435Q) were tested for their binding to CAIX using competition against biotinylated (Biotinylation kit from Innova Biosciences, Cambridge, UK) chimeric Girentuximab IgG1. An irrelevant human IgG1 antibody was tested as a negative control for binding to CAIX.

CAIX was diluted in 1×PBS to 0.5 μg/ml and 100 μl/well was coated overnight at 4 °C on a 96-well ELISA plate. The following day, the plate was washed 3× with 1×PBS/0.05% Tween (PBST) and blocked with 200 μl of 2% BSA/PBS for one hour at room temperature. In a 96-well dilution plate a fixed concentration of biotinylated Girentuximab chimeric IgG1 antibody (0.3 μg/ml final concentration) was added in equal volume to a three-fold titration series of test antibody (starting from 20 μg/ml (10 μg/ml final concentration) diluted in blocking buffer).

After washing the plate 3× with PBS-T, 100 μl of chimeric/test antibody mix was added to the ELISA plate.

After one hour incubation at room temperature, the plate was washed 3× with PBS-T and 100 μl of streptavidin-peroxidase conjugated secondary antibody (Sigma, Dorset, UK) diluted 1:1000 in PBS-T was applied for one hour at room temperature to detect bound biotinylated Girentuximab chimeric IgG1 antibody. For colour development, the plate was washed 3× with PBS-T following which 100 μl of TMB substrate was added and incubated for approximately five minutes at room temperature. The reaction was stopped with 50 μl of 3.0 M hydrochloric acid and absorbance was read immediately using a Dynex plate reader at 450 nm.

IC₅₀ values were calculated for each variant and relative IC50 values were calculated by dividing the IC50 of the humanised variant by that of the Girentuximab chimeric IgG1 antibody assayed on the same plate. All lead humanised variants and chimeric antibodies on different backbones demonstrated IC50 values within two-fold of the Girentuximab chimeric IgG1 antibody.

From analysis of all the data generated (including iTope™ analysis, percentage humanness, multicycle kinetics data and competition ELISA data), the three antibodies, VH3/Vκ4, VH4/Vκ3 and VH4V/κ4 (with sequences a provided in Table 1), were chosen as leads for further expression and analysis as IgG1, IgG1 (H310A, H435Q) and IgG4 (S228P, L235E, H310A, H435Q).

### Expression of Lead Humanised Girentuximab Antibodies in CHO Cells

Heavy chain variable domains were cloned into the Fc null vectors pANT69 (IgG1 (H310A, H435Q)) and pANT70 (IgG4 (S228P, L235E, H310A, H435Q)) as outlined above.

Chimeric (VH0/Vκ0), VH3/Vκ4, VH4/Vκ3 and VH4/Vκ4 were expressed as IgG1, IgG1 (H310A, H435Q), and IgG4 (S228, L235E, H310A, H435Q) (12 combinations in total) following transient transfection of FreeStyle™ CHO-S cells (ThermoFisher, Loughborough, UK) with corresponding plasmids and using a MaxCyte STX® electroporation system (MaxCyte Inc., Gaithersburg, USA. Following cell recovery, cells were diluted to 3×106 cells/ml in CD Opti-CHO medium (ThermoFisher, Loughborough, UK) containing 8 mM L-Glutamine (ThermoFisher, Loughborough, UK) and 1×Hypoxanthine-Thymidine (ThermoFisher, Loughborough, UK). 24 hours post-transfection, the culture temperature was reduced to 32 °C and 1 mM sodium butyrate (Sigma, Dorset, UK) was added. Cultures were fed on day 1 with 30% CD Efficient Feed B (ThermoFisher, Loughborough, UK) and 3.3% FunctionMAX™ TiterEnhancer (ThermoFisher, Loughborough, UK) and again on day 7 with 15% CD Efficient Feed B (ThermoFisher, Loughborough, UK) and 1.65% FunctionMAX™ TiterEnhancer (ThermoFisher, Loughborough, UK) (Percentages based on starting culture volumes). IgG supernatant titres were monitored by IgG ELISA and transfected cells were cultured for up to 14 days prior to harvesting supernatants.

Purification of Chimeric and Lead Humanised Antibodies Expressed as Different lgGs

The chimeric (VH0/Vκ0), VH3/Vκ4, VH4/Vκ3 and VH4/Vκ4 IgG1 antibodies and VH4/Vκ4 IgG4 (S228P, L235E) antibody were purified from cell culture supernatants using Protein A sepharose columns. Chimeric (VH0/Vκ0), VH3/Vκ4, VH4/Vκ3 and VH4/Vκ4 expressed as IgG1 (H310A, H435Q) and IgG4 (S228P, L325E, H310A, H435Q) were purified using Protein G columns. The purified antibodies were processed as outlined above. Antibodies were analysed by reducing SDS-PAGE of 2 μg loaded antibody and bands corresponding to the profile of a typical IgG were observed.

Multicycle Kinetics Analysis of Chimeric Antibodies and Lead Humanised Antibodies Expressed as Different lgGs

Multi-cycle kinetics analysis was performed on the chimeric (VH0/Vκ0), VH3/Vκ4, VH4/Vκ3 and VH4/Vκ4 antibodies on the different IgG backbones using a Biacore T200 (serial no. 1909913) instrument running Biacore T200 Evaluation Software V3.0.1 (Uppsala, Sweden) to establish an accurate affinity for carbonic anhydrase IX. The multicycle kinetics was performed as outlined in Section 2.7. VH0/Vκ0 IgG1 was run as a reference on Fc2 of each run. The relative KD was calculated by dividing the KD of the humanised variants by that of the chimeric antibody with the same IgG constant domain. The lead variants, in all three IgG backbones, all bound within two-fold of VH0/Vκ0 with the same IgG backbone.

### Conclusion and Discussion

V region genes from the Girentuximab antibody together with four humanised VH regions and five humanised Vκ regions (designed using Composite Human Antibody™ technology) were cloned into IgG1 (same allotype as Girentuximab) heavy chain and kappa light chain vectors. Chimeric (VH0/Vκ0) was additionally cloned into VH vectors IgG1 (H310A, H435Q) and IgG4 (S228P, L235E, H310A, H435Q) where mutations H310A and H435Q abolish FcRn binding.

Antibodies transiently transfected into HEK293 EBNA cells were tested for binding to CAIX by single cycle Biacore analysis. Six IgG1 lead antibodies (VH3/Vκ2, VH3/Vκ3, VH3/Vκ4, VH4/Vκ2, VH4/Vκ3 and VH4/Vκ4) were identified based on expression levels and single cycle kinetics data. Purified leads, tested by Biacore multicycle analysis and competition ELISA, were shown to bind within two-fold of the IgG1 chimeric antibody.

Based on iTope™ analysis, percentage humanness, multicycle kinetics data and competition ELISA data, the six leads were narrowed down to three; VH3/Vκ4 (SEQ ID NO: 36/SEQ ID NO: 132), VH4/Vκ3 (SEQ ID NO: 52/SEQ ID NO: 132) and VH4/Vκ4 (SEQ ID NO: 52/SEQ ID NO: 148). These three lead variants were re-cloned and antibodies were transiently expressed in CHO-S cells on three different back bones (IgG1, IgG1 (H310A, H435Q) and IgG4 (S228P, L235E, H310A, H435Q). All antibodies were purified and then tested by Biacore multicycle kinetic analysis. The lead humanised variants, in all three IgG backbones, all showed binding within two-fold of VH0/Vκ0 with the same IgG backbone.

### Example 2—Combined Targeted Radionuclide Therapy and Immune Checkpoint Inhibition

Immune checkpoint inhibition (ICI) has substantially changed cancer treatment, but (long-lasting) responses remain absent in the majority of patients. Critical determinants for successful ICI treatment are high tumor mutational burden and pre-existing tumor infiltrating lymphocytes (TIL). Ionizing radiation can trigger inflammatory signaling that increases TIL and can give rise to mutations that can be recognized by T cells. We are employing mouse models to evaluate the therapeutic action of targeted radionuclide therapy (TRT) and combined TRT+ICI therapy.

### Preparation of Radiolabelled Girentuximab Antibody Conjugates

TABLE 2
Reagents used in protocols 1 and 2

| Reagents | Source | Comment |
|---|---|---|
| Girentuximab (GmAb) antibody | Telix | GmAb supplied is TLX250 |
| | | (sequence as provided in |
| | | Table 1 eg: having VH/VL as |
| | | set forth in SEQ ID NOs 52 |
| | | and 148, respectively). |
| NHS-DOTA | Macrocylics Inc., B-280 | General grade |
| 0.25M Ammonium Acetate pH 6.0 | Sigma-Aldrich 431311 | >99.99% trace metals basis |
| 1.25M Sodium Bicarbonate pH | Sigma-Aldrich 13433 | Puriss. 99.0-100.5% |
| 8.2 | | |
| Glacial acetic acid | Fisher A507-P500 | TraceMetal grade |
| (for pH adjustment) | | |
| 0.2M NaOH | JT Baker JT-3719-03 | CMOS grade, trace impurity |
| (for pH adjustment) | | 200-10 ppb |
| Water for Injection (WFI) | Hyclone SH30221 | or equivalent grade |
| 50 kDa MWCO membrane filter | Repligen PP050LP2L membrane | TFF or Amicon spin |
| | or Amicon equivalent | concentrators |

Conjugate preparation Protocol 1: 334 μL of 1.25 M NaHCO₃, pH 8.2 was added dropwise per 20 mg of humanised girentuximab (GmAb) antibody. Next 1.25 mg NHS-DOTA was added per 20 mg of GmAb while gently swirling the mixture until NHS-DOTA was fully dissolved. The reaction mixture was incubated at 37 °C for one hour then cooled to room temperature. The GmAb-DOTA reaction solution was exchanged to 0.25M ammonium acetate pH 6.0 using a 50 kDa MWCO membrane or sufficient PD10s, up to >15× volume to ensure the removal of free NHS-DOTA in the solution. GmAb-DOTA conjugate concentration was determined by UV-vis, corrected to 5 mg/mL using 0.25 M ammonium acetate, pH 6.0.

Conjugate preparation Protocol 2: Alternatively, the conjugate may be prepared according the following “protocol 2” steps:
1. Buffer exchange GmAb into reaction buffer: 100 mM NaPO₄+20 mM EDTA pH 7.5 using a 50 kDa MWCO membrane and approximate 10× volume changes and to an approximate concentration of 5 mg/mL. Ensure pH has changed to pH 7.5.
2. Prepare an NHS-DOTA stock solution of 5 mg/ml by dissolving NHS-DOTA in COLD-Phosphate Buffered Saline, pH 7.5 no more than 30 minute prior to use i.e. use immediately. Add 12.1 molar equivalents of NHS-DOTA stock solution to the buffer-exchanged GmAb solution.
   [Alternatively, 1.25 mg solid NHS-DO TA per 20 mg of GmAb, while swirling gently mixing until solid NHS-DOTA is fully dissolved.]
3. Incubate bioconjugation reaction at 22 °C (between 21-24 °C) for two hours to allow the DOTA-conjugation reaction to run to completion.
4. Using a 50 kDa MWCO membrane; buffer exchange the GmAb-DOTA reaction solution into 0.25M ammonium acetate pH 6.0, using >15× diafiltration volumes to ensure the removal of free NHS-DOTA in the solution
5. Check final pH and protein concentration of GmAb-DOTA then concentrate to 5 mg/ml using 0.25M ammonium acetate, pH 6.0

Following either protocol 1 or 2, the produced GmAb-DOTA is exposed to a source of 177-Lu for a time sufficient for the radioisotope to chelate with the DOTA moiety. The source of 177-Lu may be any suitable source as is known in the art.

Methods: CT26 (murine colorectal carcinoma cell line) and Renca (murine renal epithelial) cells, transfected with human CAIX, were injected subcutaneously in the flank of Balb/C mice. When tumor volume reached 50-100 mm³, mice received lutetium-177-labeled CAIX-specific antibody ¹⁷⁷Lu-hG250 (intravenously) followed by ex vivo biodistribution study 24 h p.i. The antibody was prepared as described in Example 1 and 2.

The anto-PD-L1 and anti-CTLA4 antibodies used in this study were: InVivoMab anti-mouse CTLA-4 (CD152) (BioXCell, BE0032) and InVivoMab anti-mouse PD-1 (CD279) (BioXCell, BE0146).

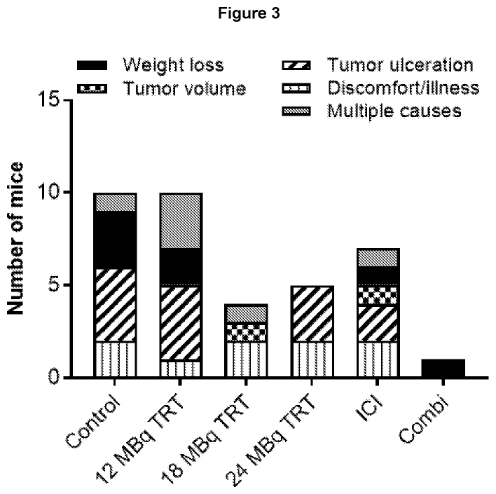

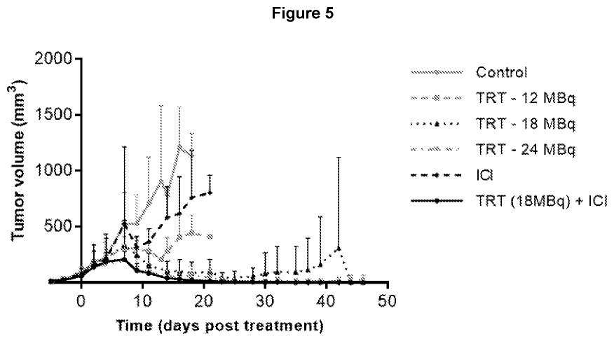

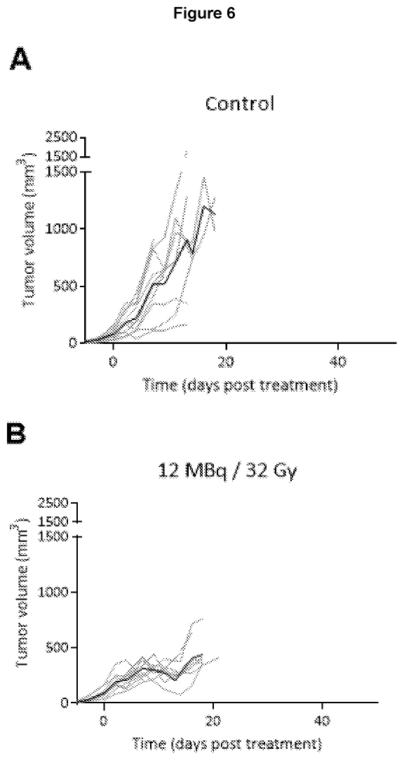

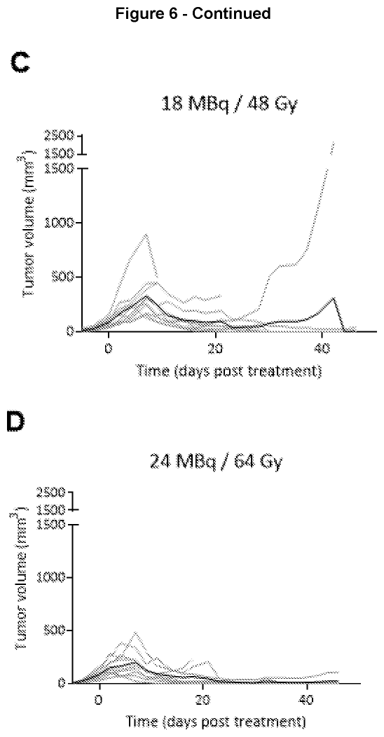

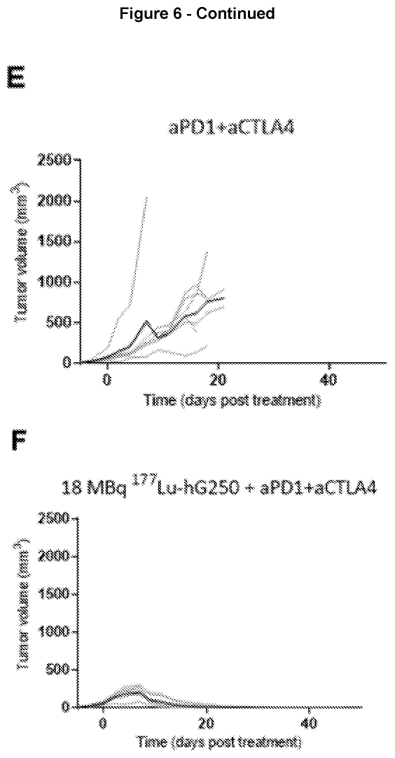

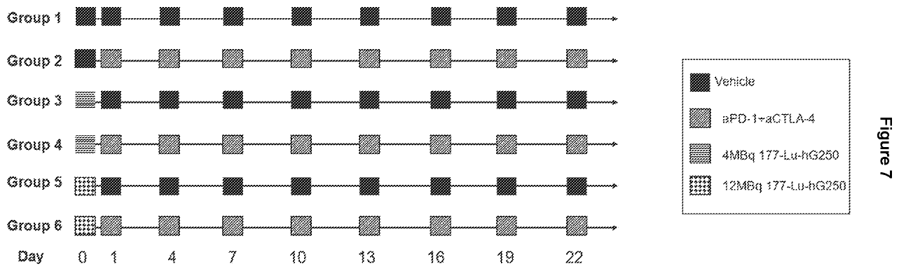

Additionally, in Renca-CAIX tumor-bearing mice, tumor growth inhibition and survival were determined upon injection of (1) 12, 18, or 24 MBq ¹⁷⁷Lu-hG250 monotherapy, (2) ICI consisting of anti-PD-1 Ab (200 μg)+anti-CTLA-4 Ab (200 μg), (3) a combination of 18 MBq ¹⁷⁷Lu-hG250 and ICI, (4) vehicle. Body mass of mice subjects were monitored (FIG. 2). Mice were euthanised according to ethical principles (FIG. 3). Tumor growth curves were quantified using the area under the curve (AUC) corrected for animal lifetime (FIGS. 1, 5 and 6). Mice that had complete regressions were re-challenged with tumor cells.

Results Tumor uptake of ¹⁷⁷Lu-hG250 was 17±2% and 32±9% ID/g in CT26-CAIX and Renca-CAIX, respectively. Immunohistochemistry (IHC) confirmed CAIX expression in both tumors. Furthermore, Renca-CAIX tumors showed high expression of PD-L1 and very few CD8′ TIL. CT26-CAIX also had prominent, but heterogeneous PD-L1 expression while tumors were heavily infiltrated by CD8+TIL.

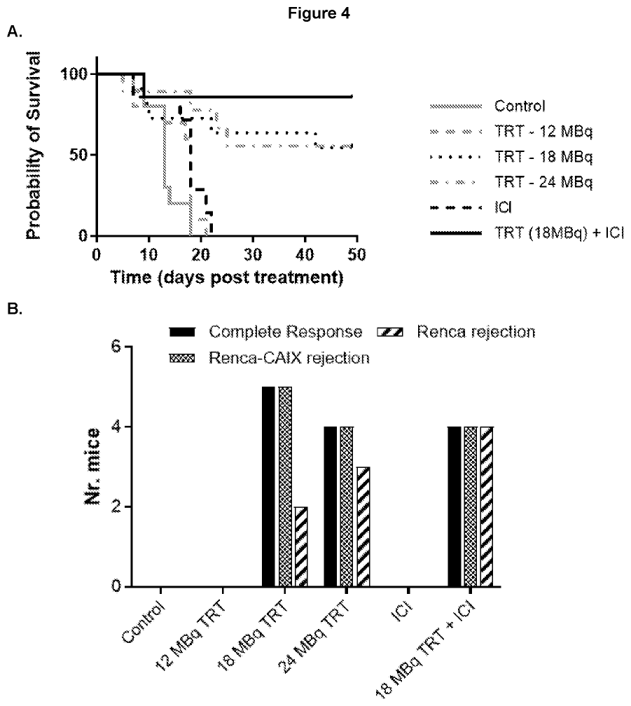

Compared to non-treated animals (AUC=449±204 mm³/day), 18 and 24 MBq ¹⁷⁷Lu-hG250 monotherapy and TRT+ICI combination therapy significantly inhibited Renca-CAIX tumor growth (AUC=202±201 mm³/day, p<0.001, AUC=95±78 mm³/day, p<0.0001 and AUC=66±64 mm³/day, p<0.0001 respectively). Additionally, these treatments significantly prolonged survival compared to non-treated animals (p<0.01, p<0.001, and p<0.005 respectively) (FIG. 4). Complete tumor regression was observed in 50%, 40%, and 57% of the mice, respectively. Re-challenge of these animals with tumor cells resulted in 100% rejection of Renca-CAIX in all groups. In the TRT+ICI combination therapy group, 100% of parental Renca tumors were also rejected, whereas 40% and 75% were rejected in 18 and 24 MBq TRT monotherapy groups, respectively.

Conclusion: TRT with ¹⁷⁷Lu-hG250 as well as its combination with ICI was therapeutically efficacious. Combination of TRT with ICI enables comparable anti-tumoral efficacy with lower doses of TRT compared to TRT alone. Tumor re-challenge experiments suggest the induction of tumor-specific T cell responses following TRT.

### Example 3—Combined Targeted Radionuclide Therapy and Immune Checkpoint Inhibition

A study similar to that described in Example 2 was conducted using a lower dose of targeted radiotherapy (TRT), alone or in combination with ICI treatment. Briefly, Renca cells, transfected with human CAIX, were injected subcutaneously in the flank of Balb/C mice. When tumor volume reached 50-100 mm³, mice received lutetium-177-labeled CAIX-specific antibody ¹⁷⁷Lu-hG250 (intravenously) followed by anti-PD-1 and anti-CTLA4 antibody.

The experimental design is summarised in FIG. 7. Briefly, mice were split into the following treatment groups:
1. Control (vehicle+vehicle)
2. ICI monotherapy (vehicle+PD-1 Ab+anti-CTLA-4 Ab);
3. TRT low dose monotherapy (4 MBq ¹⁷⁷Lu-hG250+vehicle);
4. TRT+ICI low dose therapy (4 MBq ¹⁷⁷Lu-hG250+PD-1 Ab+anti-CTLA-4 Ab)
5. TRT mid dose monotherapy (12 MBq ¹⁷⁷Lu-hG250+vehicle);
6. TRT+ICI mid dose therapy (12 MBq ¹⁷⁷Lu-hG250+PD-1 Ab+anti-CTLA-4 Ab)

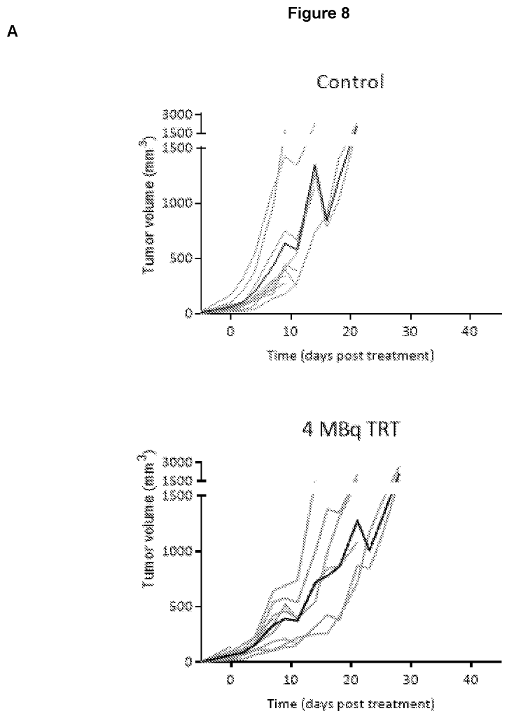

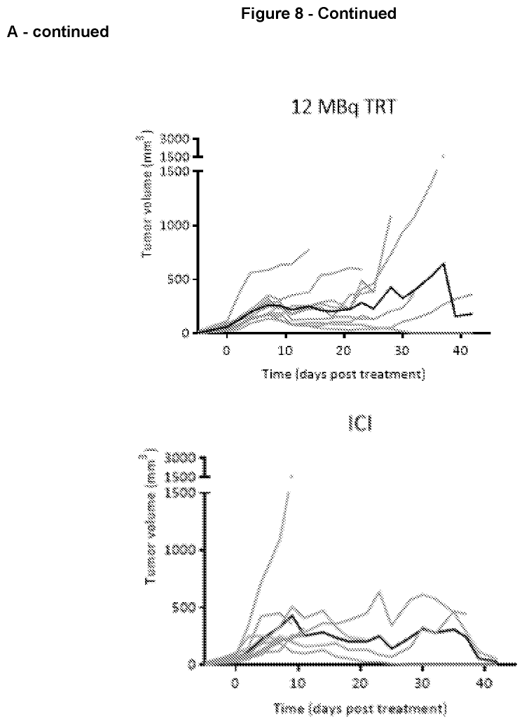

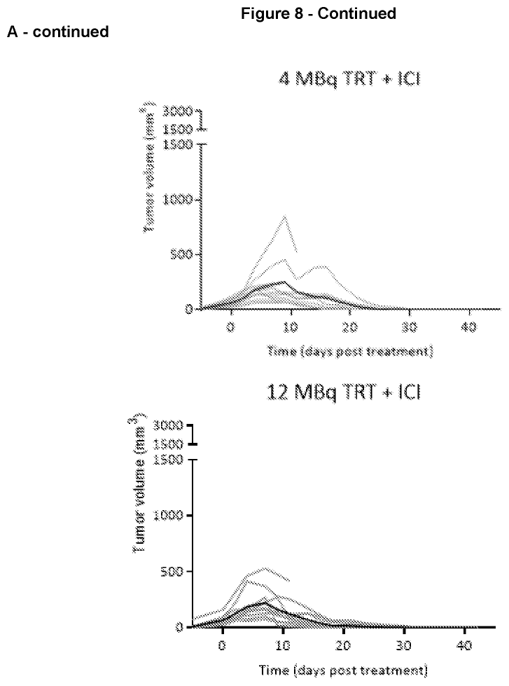

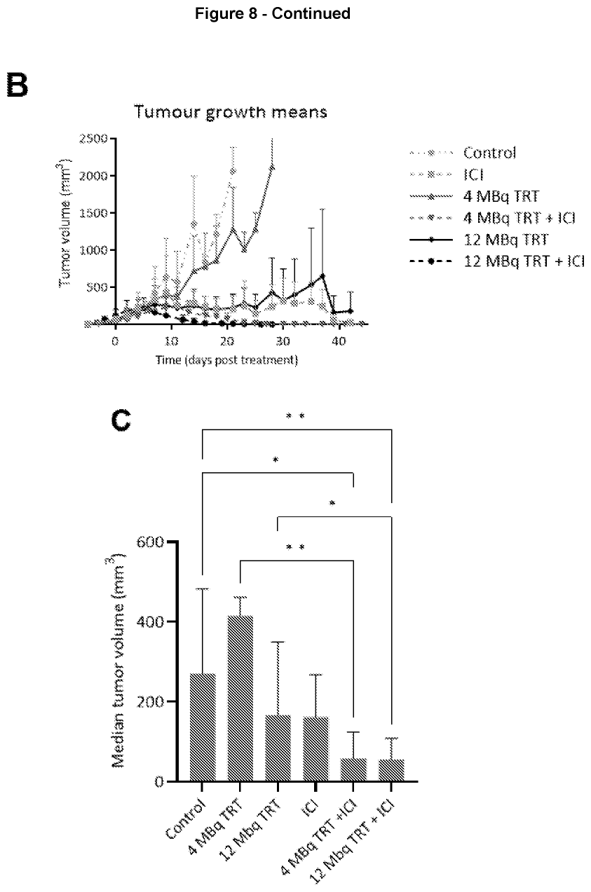

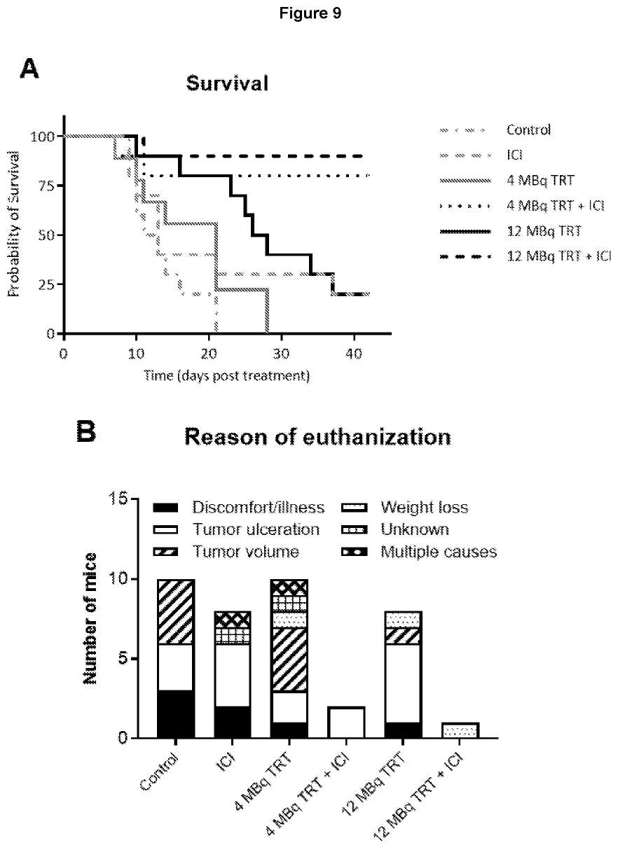

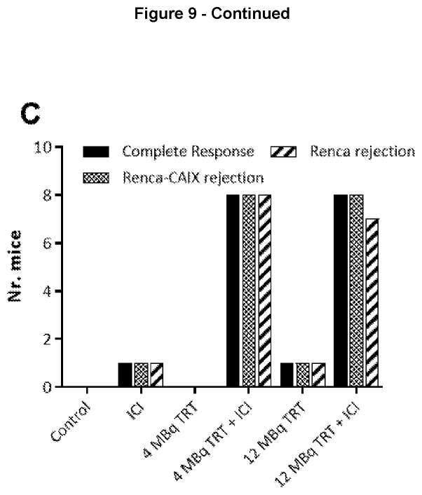

Inhibition of tumor growth and survival were determined and the results are shown in FIGS. 8 and 9. Briefly, the results are consistent with the observations made in Example 2, and show that combination TRT and ICI, including at lower doses of radiation compared to Example 2, show significant reduction in tumor growth compared to control or monotherapy (either TRT or ICI alone).

| Treatment | % alive at end of study | % complete response |
|---|---|---|
| Control | 0 | 0 |
| ICI | 20 | 10 |
| 4 MBq TRT | 0 | 0 |
| 4 MBq TRT + ICI | 80 | 80 |
| 12 MBq TRT | 20 | 10 |
| 12 MBq TRT + ICI | 90 | 80 |

Mice that had complete regressions were re-challenged with tumor cells—either with the “parent” Renca cell line on one flank, and on the other flank, Renca-CAIX which they had received in the primary experiment.

The below table shows that a high proportion of animals treated with TRT+ICI combination therapy had complete tumor regression, and almost all these animals rejected a tumor-rechallenge. These results indicate that the animals had induced tumor-specific memory T cell responses.

| | # rechallenged | Renca-CAIX | Renca tumour |
|---|---|---|---|
| Treatment | (out of 10) | tumour rejection | rejection |
| Control | 0 | | |
| ICI | 1 | 1 (100%) | 1 (100%) |
| 4 MBq TRT | 0 | | |
| 4 MBq TRT + ICI | 8 | 8 (100%) | 8 (100%) |
| 12 MBq TRT | 1 | 1 (100%) | 1 (100%) |
| 12 MBq TRT | 8 | 8 (100%) | 7 (87.5%) |

### Example 4—Combined Targeted Radionuclide Therapy and Immune Checkpoint Inhibition (CT26 Model)

A study similar to that described in Example 3 was conducted using the CT26-huCAIX model. CT26 cells, transfected with human CAIX, were injected subcutaneously in the flank of Balb/C mice. When tumor volume reached 50-100 mm³, mice received lutetium-177-labeled CAIX-specific antibody ¹⁷⁷Lu-hG250 (intravenously) followed by anti-PD-1 antibody.

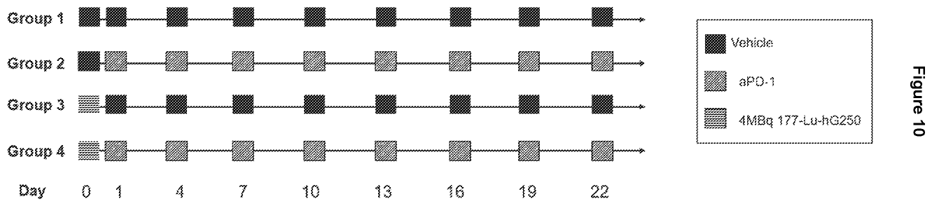

The experimental design is summarised in FIG. 10. Briefly, mice were split into the following treatment groups:
1. Control (vehicle+vehicle)
2. ICI monotherapy (vehicle+anti-PD-1 Ab)
3. TRT low dose monotherapy (4 MBq ¹⁷⁷Lu-hG250+vehicle)
4. TRT+ICI low dose therapy (4 MBq ¹⁷⁷Lu-hG250+anti-PD-1 Ab)

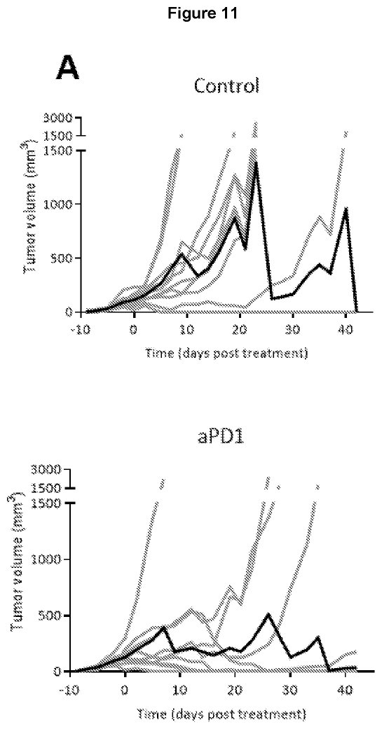

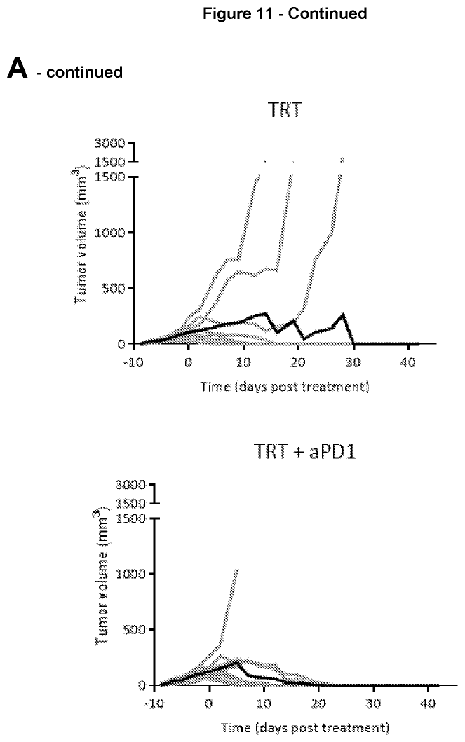

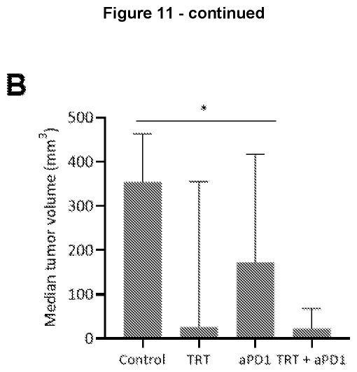

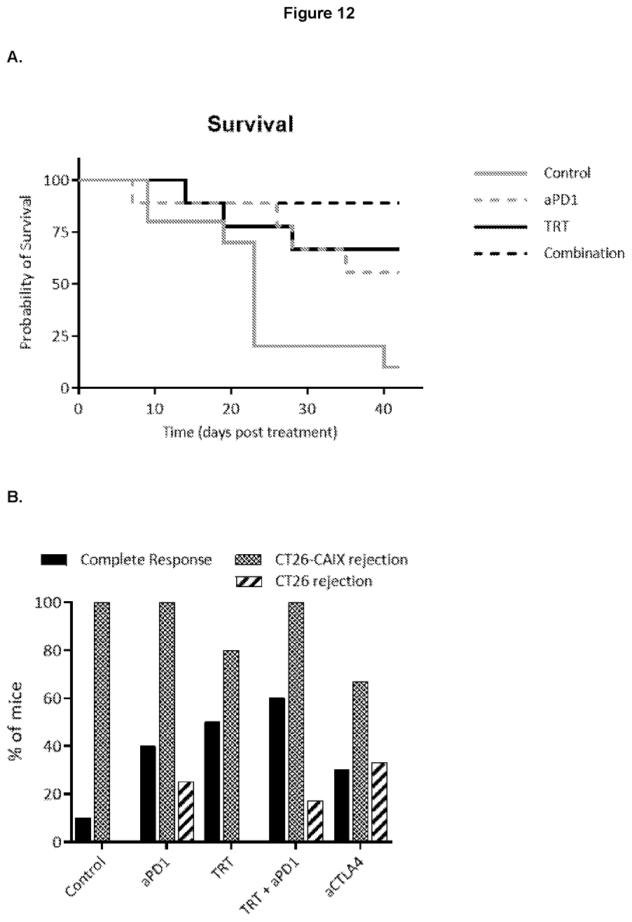

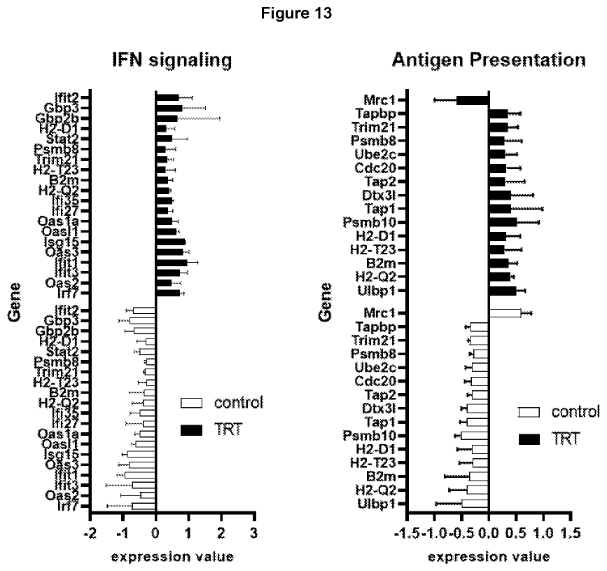

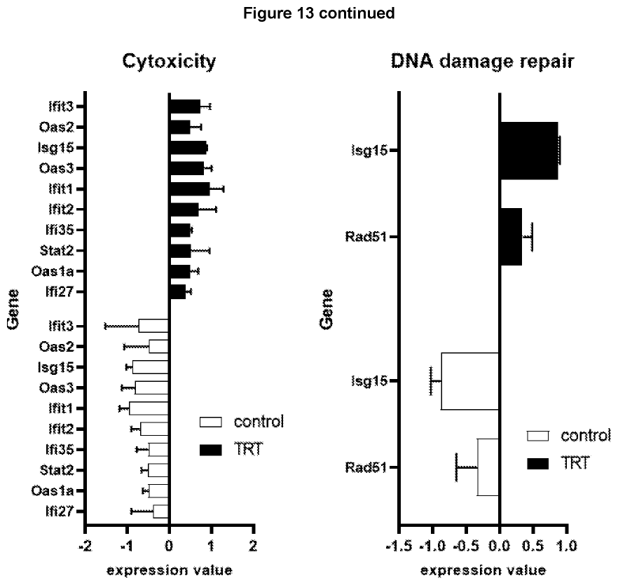

Inhibition of tumor growth and survival were determined and the results are shown in FIGS. 11 and 12. Briefly, the results are consistent with the observations made in Example 2, and show that combination TRT and ICI, including at lower doses of radiation compared to Example 2, show significant reduction in tumor growth compared to control or monotherapy (either TRT or ICI alone).

### Example 5—Assessment of Changes in Gene Expression Following 177-Lu-hG250 Treatment

In order to better understand the mechanism of action by which 177-Lu-hG250 treatment modulates the tumour microenvironment to increase the efficacy of ICI treatment, the inventors performed gene expression profiling of Renca-hCAIX tumour bearing mice using the 10360 PanCancer panel from the Nanostring platform.

TRT groups were treated with 177-Lu-hG250, 12MBq of radiation. Tumours were harvested 7 days later for gene expression analysis.

The results are shown in Example 13 and indicate that 177-Lu-hG250 treatment induces expression of several genes involved in processes that facilitate anti-tumour immunity and that are associated with response to checkpoint inhibition, including genes associated with interferon (IFN) signalling, antigen presentation, cytotoxicity and DNA damage repair.

Graphs are derived from a list of genes passing a filter of at least 1.5 fold differential expression and p value<0.05. N=3 mice per group. Expression values represent the mean subtracted normalized log 2 values.

### Example 6—Cellular Profiling and Assessment of Changes in Gene Expression Following 177-Lu-hG250 Treatment

Further investigations are carried out in order to better understand the mechanism of action by which 177-Lu-hG250 treatment modulates to tumour microenvironment to increase the efficacy of ICI treatment. In this series of experiments, a lower dosage of 177-Lu-hG250 treatment was administered compared to Example 4 (eg 4 MBq 177-Lu-hG250 as compared with 12 MBq 177-Lu-hG250 in Example 4).

The experimental protocol is outlined in FIG. 14. Briefly, Renca-hCAIX expressing mice are treated according to the following protocols:

### Part 1:

### Animal Groups

Control (no treatment)
4MBq 177Lu-hG250
anti-PD-1+anti-CTLA-4 antibodies
4MBq 177Lu-hG250+anti-PD-1+anti-CTLA-4 antibodies

Tumours are harvested at either days 0, ~D5, ~D8 post treatment initiation. Flow cytometry is conducted to profile immune cells in the tumour microenvironment including markers of Effector T cells, T cell activation/exhaustion, Regulatory T cells, Myeloid cells, Tumour-antigen-specific T cells.

The results will show that low dose targeted radiation/low dose targeted radiation in combination with checkpoint inhibition changes the types/proportions of immune cells in a manner that promotes anti-tumor immunity. For example, an increase in activation status of effector T cells and decreases in suppressive immune cells types will be seen.

### Part 2:

### Animal Groups:

Control (no treatment)
4MBq 177Lu-hG250
anti-PD-1+anti-CTLA-4 antibodies
4MBq 177Lu-hG250+anti-PD-1+anti-CTLA-4 antibodies

Tumours are harvested at either DO, ~D5, ~D8 post treatment initiation and gene expression profiling is conducted using nanostring to assess changes in the expression of >700 Immunology genes. In addition, immunohistochemistry of T cell and myeloid markers is performed to assess the effect of combination 177Lu-hG250, anti-PD-1+anti-CTLA-4 antibodies on the tumour microenvironment.

The results will show that low dose targeted radiation/low dose targeted radiation in combination with checkpoint inhibition changes the gene expression profile of cells in the tumor microenvironment that promotes anti-tumor immunity, similar to the observations in example 4. For example, the results will show an increase in the expression of genes involved in IFN signalling, T cell chemo-attraction, cytotoxicity and antigen presentation.

### EMBODIMENTS

1. A method of treating, preventing or minimising progression of cancer in a subject, the method comprising:
   administering to the subject a therapeutically effective amount of an antigen binding protein that binds to or specifically binds to carbonic anhydrase IX (CAIX), wherein the antigen binding protein conjugated with a radionuclide,
   in combination with immune checkpoint inhibitor therapy,
   thereby treating, preventing or minimising progression of cancer in the subject.
2. The method of embodiment 1, wherein the antigen binding protein comprises an antigen binding domain that binds specifically to carbonic anhydrase IX (CAIX) and comprises:

FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1.
3. The method of embodiment 2, wherein the antigen binding domain comprises:
   (i) a VH comprising complementarity determining region (CDR) 1 comprising or consisting of a sequence as set forth in SEQ ID NO: 1, a CDR2 comprising or consisting of a sequence as set forth in SEQ ID NO: 2 and a CDR3 comprising or consisting of a sequence as set forth in SEQ ID NO: 3; and
   (ii) a VL comprising complementarity determining region (CDR) 1 comprising or consisting of a sequence as set forth in SEQ ID NO: 81, a CDR2 comprising or consisting of a sequence as set forth in SEQ ID NO: 82 and a CDR3 comprising or consisting of a sequence as set forth in SEQ ID NO: 83;
or
   (iii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence set forth in SEQ ID NO: 1, a CDR2 comprising or consisting of a sequence at least about 80%, at least 85%, at least 90%, at least 92%, at least 95%, at least 97%, at least 99% identical to a sequence set in SEQ ID NO: 2, and a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence set forth in SEQ ID NO: 3; and
   (iv) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence set forth in SEQ ID NO: 81, a CDR2 comprising or consisting of a sequence at least about 80%, at least 85%, at least 90%, at least 92%, at least 95%, at least 97%, at least 99% identical to a sequence set in SEQ ID NO: 82, and a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence set forth in SEQ ID NO: 83.
4. The method of embodiment 2 or 3, wherein the antigen binding domain comprises:
   (i) a VH comprising a framework region (FR) 1 comprising or consisting of a sequence as set forth in SEQ ID Nos: 9, 25, 41, 57 or 73; a FR2 comprising or consisting of a sequence as set forth in SEQ ID Nos: 10, 26, 42, 58 or 74; a FR3 comprising or consisting of a sequence as set forth in SEQ ID Nos: 11, 27, 43, 59 or 75; a FR4 comprising or consisting of a sequence as set forth in SEQ ID Nos: 12, 28, 44, 60 or 76; and
   (ii) a VL comprising a framework region (FR) 1 comprising or consisting of a sequence as set forth in SEQ ID Nos: 89, 105, 121, 137, 153 or 169; a FR2 comprising or consisting of a sequence as set forth in SEQ ID Nos: 90, 106, 122, 138, 154 or 170; a FR3 comprising or consisting of a sequence as set forth in SEQ ID Nos: 91, 107, 123, 139, 155 or 171; a FR4 comprising or consisting of a sequence as set forth in SEQ ID Nos: 92, 108, 124, 140, 156 or 172;
or
   (iii) a VH comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID Nos: 9, 25, 41, 57 or 73; a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID Nos: 10, 26, 42, 58 or 74; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID Nos: 11, 27, 43, 59 or 75; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID Nos: 12, 28, 44, 60 or 76; and
   (iv) a VL comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID Nos: 89, 105, 121, 137, 153 or 169; a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID Nos: 90, 106, 122, 138, 154 or 170; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID Nos: 91, 107, 123, 139, 155 or 171; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID Nos: 92, 108, 124, 140, 156 or 1722.
5. The method of any one of claims 1-4, wherein the antigen binding protein comprises:
(a) a heavy chain variable domain (VH) comprising an amino acid sequence as set forth in SEQ ID NOs: 4, 20, 36, 52 or 68 and/or (b) a light chain variable domain (VL) comprising an amino acid sequence as set forth in SEQ ID NOs: 84, 100, 116, 132, 148 or 164;
or
(c) a heavy chain variable domain (VH) comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NOs: 4, 20, 36, 52 or 68 and/or (d) a light chain variable domain (VL) comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in SEQ ID NOs: 84, 100, 116, 132, 148 or 164.
6. The method of any one of embodiments 1-5, wherein the antigen binding protein is an antibody or antigen binding fragment thereof.
7. The method of embodiment 1, wherein the antigen binding protein is the antibody girentuximab.
8. The method of any of embodiments 1-6, wherein the antigen binding protein is a modified antibody of class IgG, comprising a heavy chain constant region having one or more amino acid substitutions compared to a wild-type antibody of the class IgG, wherein the one or more amino acid substitutions reduce the affinity of the antibody for the neonatal Fc receptor (FcRn), thereby reducing the serum half-life of the modified monoclonal antibody compared to a wild-type antibody of class IgG.
9. The method of embodiment 8, wherein the one or more amino acid substitutions are selected from substitutions in the heavy chain constant region at one or more of positions His310, His433, His435, His436, Ile253.
10. The method of embodiment 9, wherein the one or more amino acid substitutions comprise a substitution in the heavy chain constant region at position His310 or at His435, preferably, wherein the amino acid substitutions are at both His310 and His435.
11. The method of any one of embodiments 8-10, wherein the modified antibody further comprises: (a) one or more amino acid substitutions that reduce the affinity of the antibody for one or more Fc gamma receptors compared to a wild-type antibody of the class IgG; and/or (b) one or more amino acid substitutions that increase the stability of the CH1-CH2 hinge region in the modified antibody compared to a wild-type antibody of the class IgG.
12. The method of embodiment 11, wherein the one or more amino acid substitutions that reduce affinity of the antibody for an Fc gamma receptor comprises an amino acid substitution at position Leu235.
13. The method of embodiment 11 or 12, wherein the one or more amino acid substitutions that increase the stability of the CH1-CH2 hinge region in the antibody comprises a substitution at Ser228.
14. The method of any one of embodiments 8-13, wherein the modified antibody is conjugated to the radionuclide via a linker or chelator moiety.
15. The method of any of embodiments 1-14 wherein the radioisotope is selected from the group consisting of: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 and radium-224 (²²³Ra, ²²4Ra), samarium-153 (¹⁵³Sm), scandium-47 (⁴⁷Sc), strontium-90 (⁹⁰Sr), and yttrium-90 (⁹⁰Y).
16. The method of any one of embodiments 1-15, wherein the antigen binding protein is a modified antibody with reduced FcRn binding affinity compared to that of an unmodified form of the antibody, or to a wild-type antibody of the class IgG, comprising:
   a heavy chain constant region wherein one or more amino acid residues at positions His310, His433, His435, His 436, and Ile253 are different from the residues present in the unmodified antibody, or a wild-type antibody of the class IgG.
17. The method of any one of embodiments 1 to 16, wherein the antigen binding protein comprises a heavy chain comprising the sequence set forth in any one of SEQ ID NOs: 182 to 185; and/or a light chain constant region comprising the amino acid sequence as set forth in SEQ ID NO: 181 or SEQ ID NO: 186.
18. The method of embodiment 16 wherein the antibody comprises the amino acid sequences as set forth in SEQ ID NO: 183 and 186.
19. The method of embodiment 1, wherein the antigen binding protein is a molecule comprising an immunoglobulin moiety and a non-protein agent conjugated thereto,
   wherein, the immunoglobulin moiety specifically binds to CAIX and comprises:
   an antigen binding protein that consists essentially of or consists of an amino acid sequence of (in order of N to C terminus or C to N terminus) any one of SEQ ID NOs: 4, 20, 36, 52 or 68 and any one of SEQ ID Nos 84, 100, 116, 132, 148, 164, wherein the immunoglobulin moiety has reduced or abolished affinity for the FcRn receptor compared to a wild-type immunoglobulin; and
   wherein the non-protein agent comprises a radioactive element.
20. The method of any one of the preceding embodiments, wherein the radionuclide is selected from: actinium-225 (²²⁵Ac), astatine-211 (²¹¹At), and iodine-123 (¹²³I)
21. The method of any one of the preceding embodiments, wherein the antigen binding protein is a therapeutic IgG molecule comprising:
   heavy and light chains, wherein each chain comprises a variable region and a constant region;
   wherein the constant region of the heavy chain comprises one or more amino acid substitutions compared to a wild-type antibody of the class IgG, wherein the one or more amino acid substitutions reduce the affinity of the antibody for the neonatal Fc receptor (FcRn), thereby reducing the serum half-life of the modified monoclonal antibody compared to a wild-type antibody of class IgG; and
   a radioisotope conjugated to one or more amino acid residues of the heavy or light chains.
22. The method of any one of the preceding embodiments wherein the antigen binding protein is an antibody, preferably a naked antibody.
23. The method according to any one of the preceding embodiment, wherein the radionuclide is ¹⁷⁷Lu.
24. The method of any one of the preceding embodiments, wherein the radioactivity of the antigen binding protein for binding to CAIX conjugated with a radionuclide is not more than about 18MBq.
25. The method of any one of the preceding embodiments, wherein the radionuclide is conjugated to the antibody for binding to CAIX through a linker or a chelator.
26. The method of embodiment 25, wherein the chelator is selected from: TMT (6,6″-bis[N,N″,N″′-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N″-tetraacetic acid), TCMC, DO3A, CB-DO2A, NOTA, Diamsar, DTPA, CHX-A″-DTPA, TETE, Te2A, HBED, DFO, DFOsq and HOPO.
27. The method of any one of the preceding embodiments 1-68, further comprising, prior to the administering step, conjugating the antigen binding protein with the radionuclide.
28. The method of embodiment 27, wherein the antigen binding protein is a modified antibody for binding to CAIX, and the method further comprises, prior to the conjugating step, generating the modified antibody for binding to CAIX comprising a non-peptide moiety.
29. The method of embodiment 28, wherein the non-peptide moiety is a linker or chelator.
30. The method of embodiment 29, wherein the chelator is selected from the group consisting of: TMT (6,6″-bis[N,N″,N″-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N″′-tetraacetic acid), TCMC, DO3A, CB-DO2A, NOTA, Diamsar, DTPA, CHX-A″-DTPA, TETE, Te2A, HBED, DFO, DFOsq and HOPO.
31. The method of any one of the preceding embodiments, wherein the immune checkpoint inhibitor therapy comprises targeting a protein selected from: PD-1, PD-L1 and CTLA-4, or a combination thereof.
32. The method of embodiment 31, wherein the immune checkpoint inhibitor therapy comprises targeting PD-1 and CTLA4.
33. The method of embodiment 32, wherein the immune checkpoint inhibitor therapy comprises administration of an antibody for binding to PD-1 and an antibody for binding to CTLA4 to the subject.
34. The method of any one of the preceding embodiments, the method further comprising administering one or more immune checkpoint inhibitors to the subject.
35. The method of embodiment 34, wherein the administration of the antigen binding protein and the immune checkpoint inhibitor is separate, consecutive or sequential.
36. A method of inducing a T-cell response to a cancer in a subject, comprising administering an effective amount of an antibody or antigen binding fragment thereof to CAIX to the subject in combination with an immune checkpoint inhibitor, thereby inducing the T cell response to the cancer in the subject.
37. A method of inducing an adaptive immune response to a cancer in a subject, comprising administering an effective amount of an antibody or antigen binding fragment thereof to CAIX to the subject in combination with an immune checkpoint inhibitor, thereby inducing a T cell response to the cancer in the subject.
38. The method of any one of one of the preceding embodiments, wherein the cancer is renal cancer.
39. Use of an antigen binding protein for binding to CAIX in the manufacture of a medicament for use in combination with immune checkpoint inhibitor therapy for the treatment of cancer, preferably renal carcinoma.
40. Use of an immune checkpoint inhibitor in the manufacture of a medicament for use, in combination with an antigen binding protein for binding to CAIX conjugated with a radionuclide, in the treatment of cancer, preferably renal carcinoma.
41. An antigen binding protein for binding to CAIX for use, in combination with immune checkpoint inhibitor therapy, in the treatment of cancer, preferably renal carcinoma.
42. An immune checkpoint inhibitor for use in the treatment of cancer, preferably renal carcinoma, in combination with an antigen binding protein for binding to CAIX conjugated with a radionuclide.
43. A pharmaceutical composition comprising an antigen binding protein for binding to CAIX, wherein the pharmaceutical composition is for use in the treatment of cancer, preferably renal carcinoma, in combination with an immune checkpoint inhibitor.
44. A pharmaceutical composition comprising an immune checkpoint inhibitor for use in the treatment of cancer, preferably renal carcinoma, in combination with an antigen binding protein for binding to CAIX conjugated with a radionuclide.
45. A kit for use or when used in accordance with any method described herein, the kit comprising in separate parts:
   an antigen binding protein for binding to CAIX, optionally conjugated with a radionuclide; and
   instructions for its use in combination with an immune checkpoint inhibitor.
46. The kit of embodiment 87, further comprising, in a separate part, one or more immune checkpoint inhibitor(s).
47. A kit for use or when used in accordance with any method described herein, the kit comprising:
   one or more immune checkpoint inhibitors; and
   instructions for its use in combination with an antigen binding protein for binding to CAIX conjugated with a radionuclide.
48. The kit of embodiment 47, further comprising, in a separate part, an antigen binding protein for binding to CAIX optionally conjugated with a radionuclide.
49. The kit of any one of embodiments 45-48, wherein the antigen binding protein for binding to CAIX and/or the immune checkpoint inhibitor are provided in the form of a pharmaceutical composition optionally comprising one or more pharmaceutically acceptable excipients, carriers and/or diluents.

1. A method of:
(a) treating, preventing or minimizing progression of cancer in a subject;
(b) inducing a T-cell response to a cancer in a subject; and/or
(c) inducing an adaptive immune response to a cancer in a subject;
the method comprising:
administering to the subject: a radiolabeled agent that binds to or specifically binds to carbonic anhydrase IX (CAIX), and an immune checkpoint inhibitor,
thereby treating, preventing or minimizing progression of cancer in the subject, inducing a T-cell response to a cancer in the subject, and/or inducing an adaptive immune response to a cancer in the subject.

2.-3. (canceled)

4. A method of treating, preventing or minimising the progression of cancer in a subject who has received, or who is receiving treatment with an immune checkpoint inhibitor, the method comprising:
administering to the subject a radiolabelled agent that binds to or specifically binds to CAIX,
thereby treating, preventing or minimising progression of cancer in the subject.

5. The method of claim 1, wherein:
(i) the radiolabeled agent is selected from a small molecule, peptide or protein, capable of binding to or specifically binding to CAIX;
(ii) the radiolabelled agent is a radiolabelled peptide:
(iii) the radiolabelled agent is an antibody or antigen binding fragment thereof capable of binding to or specifically binding to CAIX, and wherein the antibody or antigen binding fragment thereof is conjugated to a radionuclide:
(iv) the radiolabelled agent is conjugated to a radionuclide selected from the group consisting of: actinium-225 (225Ac), astatine-211 (211At), bismuth-212 and bismuth-213 (212Bi, 213Bi), copper-64 and copper-67 (64Cu, 67Cu), iodine-123, -124, -125 or -131 (123I, 124I, 125I, 131I), lead-212 (212Pb), lutetium-177 (177Lu), radium-223 and radium-224 (223Ra, 224Ra), rhenium-186 and rhenium-188 (186Re and 188Re), samarium-153 (153Sm), and scandium-47 (47Sc), strontium-90 (90Sr), terbium-149 and terbium-161 (149Tb and 161Tb), and yttrium-90 (90Y); and/or
(v) the agent is conjugated to a radionuclide prior to administration.

6.-7. (canceled)

8. The method of claim 5, wherein the radiolabelled antibody or antigen binding fragment thereof is a radiolabelled girentuximab antibody, or antigen binding fragment thereof.

9. The method of claim 5, wherein the radiolabelled antibody or antigen binding fragment thereof comprises an antigen binding domain that binds to or specifically binds to CAIX and wherein the antigen binding domain comprises:
(a) the three CDRs of an antigen binding domain having a heavy chain variable domain (VH) as set forth in SEQ ID NOs: 4, 20, 36, 52 or 68; and/or
(b) the three CDRs of an antigen binding domain having a light chain variable domain (VL) comprising the amino acid sequence as set forth in SEQ ID NOs: 84, 100, 116, 132, 148 or 164;
preferably wherein the complementarity determining regions have an amino acid sequence of any of SEQ ID NOs: 1-3 and 97-99.

10. The method of claim 5, wherein the antigen binding domain comprises:
(A)
(i) a VH comprising a complementarity determining region (CDR) 1 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 1, a CDR2 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set in SEQ ID NO: 2, and a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 3;
(ii) a VH comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in any of SEQ ID NOs: 4, 20, 36, 52 or 68;
(iii) a VL comprising a CDR1 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 81, a CDR2 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 82 and a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NO: 83;
(iv) a VL comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence set forth in SEQ ID NOs: 84, 100, 116, 132, 148 or 164;
(v) a VH comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3;
(vi) a VH comprising a sequence set forth in any of SEQ ID NOs: 4, 20, 36, 52 or 68;
(vii) a VL comprising a CDR1 comprising a sequence set forth in SEQ ID NO: 81, a CDR2 comprising a sequence set forth in SEQ ID NO: 82, and a CDR3 comprising a sequence set forth in SEQ ID NO: 83;
(viii) a VL comprising a sequence set forth in any of SEQ ID NOs: 84, 100, 116, 132, 148 or 164;
(ix) a VH comprising a CDR1 comprising a sequence set forth in SEQ ID NO: 1, a CDR2 comprising a sequence set forth between in SEQ ID NO: 2, and a CDR3 comprising a sequence set forth in SEQ ID NO: 3; and a VL comprising a CDR1 comprising a sequence set SEQ ID NO: 81, a CDR2 comprising a sequence set forth in SEQ ID NO: 82, and a CDR3 comprising a sequence set forth in SEQ ID NO: 83; or
(x) a VH comprising a sequence set forth in any of SEQ ID NOs: 4, 20, 36, 52 or 68 and a VL comprising a sequence set forth in any of SEQ ID NOs: 84, 100, 116, 132, 148 or 164, preferably wherein the VH comprises the sequence as set forth in SEQ ID NO: 4 and the VL comprises the sequence as set forth in SEQ ID NO: 100, or wherein the VH comprises the sequence as set forth in SEQ ID NO: 52 and the VL comprises the sequence as set forth in SEQ ID NO: 148;
(B)
(i) a VH comprising a framework region (FR) 1 comprising or consisting of a sequence as set forth in any of SEQ ID NOs: 9, 25, 41, 57 or 73; a FR2 comprising or consisting of a sequence as set forth in any of SEQ ID NOs: 10, 26, 42, 58 or 74: a FR3 comprising or consisting of a sequence as set forth in any of SEQ ID NOs: 11, 27, 43, 59 or 75: a FR4 comprising or consisting of a sequence as set forth in any of SEQ ID Nos: 12, 28, 44, 60 or 76, and
(ii) a VL comprising a framework region (FR) 1 comprising or consisting of a sequence as set forth in any of SEQ ID NOs: 89, 105, 121, 137, 153 or 169; a FR2 comprising or consisting of a sequence as set forth in any of SEQ ID NOs: 90, 106, 122, 138, 154 or 170: a FR3 comprising or consisting of a sequence as set forth in any of SEQ ID NOs: 91, 107, 123, 139, 155 or 171; a FR4 comprising or consisting of a sequence as set forth in any of SEQ ID NOs: 92, 108, 124, 140, 156 or 172:
or
(iii) a VH comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 9, 25, 41, 57 or 73: a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 10, 26, 42, 58 or 74; a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 11, 27, 43, 59 or 75; a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 12, 28, 44, 60 or 76, and
(iv) a VL comprising a framework region (FR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 89, 105, 121, 137, 153 or 169: a FR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 90, 106, 122, 138, 154 or 170: a FR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 91, 107, 123, 139, 155 or 171: a FR4 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 92, 108, 124, 140, 156 or 172;
(C)
(i) a heavy chain variable domain (VH) comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 4, 20, 36, 52 or 68; and/or
(ii) a light chain variable domain (VL) comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% identical to a sequence as set forth in any of SEQ ID NOs: 84, 100, 116, 132, 148 or 164; and/or
(i) a heavy chain variable domain (VH) comprising an amino acid sequence as set forth in any of SEQ ID NOs: 4, 20, 36, 52 or 68; and/or
(ii) a light chain variable domain (VL) comprising an amino acid sequence as set forth in any of SEQ ID NOs: 84, 100, 116, 132, 148 or 164.

11.-12. (canceled)

13. The method of claim 5,
wherein the antibody or antigen binding fragment thereof comprises the amino acid sequence of a VH and a VL selected from: SEQ ID NO: 4 and SEQ ID NO: 84; or SEQ ID NO: 36 and SEQ ID NO: 132; or SEQ ID NO: 52 and SEQ ID NO: 132; or SEQ ID NO: 52 and SEQ ID NO: 148.

14. The method of claim 5, wherein the antibody or antigen binding fragment thereof is a modified antibody of class IgG, comprising a heavy chain constant region having one or more amino acid substitutions compared to a wild-type antibody of the class IgG, wherein the one or more amino acid substitutions reduce the affinity of the antibody for the neonatal Fc receptor (FcRn), thereby reducing the serum half-life of the modified monoclonal antibody compared to a wild-type antibody of class IgG;
optionally wherein the antibody or antigen binding fragment thereof is a modified antibody with reduced FcRn binding affinity compared to that of an unmodified form of the antibody, or to a wild-type antibody of the class IqG, comprising a heavy chain constant region wherein one or more amino acid residues at positions His310, His433, His435, His 436, and Ile253 are different from the residues present in the unmodified antibody, or a wild-type antibody of the class IgG.

15.-16. (canceled)

17. The method of claim 5, wherein the radionuclide is lutetium-177 (¹⁷⁷Lu) or actinium-225 (²²⁵Ac).

18. The method of claim 5, wherein;
(i) the radionuclide is ¹⁷⁷Lu; and
(ii) the immune checkpoint inhibitor therapy comprises administration to the subject, an antibody for binding to PD-1 and/or an antibody for binding to CTLA4.

19. The method of claim 1, wherein:
(i) the radioactivity of the agent conjugated to theradionuclide is not more than about 18 MBq, optionally wherein the radioactivity is not more than about 12 MBq; and
(ii) the radiolabelled agent comprises a radionuclide conjugated to a peptide or antibody or antigen binding fragment thereof via a linker or a chelator, preferably wherein the chelator is selected from: TMT (6,6″-bis[N,N″,N′-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N′-tetraacetic acid), TCMC, DO3A, CB-DO2A, NOTA, Diamsar, DTPA, CHX-A″-DTPA, TETE, Te2A, HBED, DFO, DFOsq, DFO-NCS, HOPO or a chelator as described in WO 2022/133537.

20.-22. (canceled)

23. The method of claim 1, wherein the immune checkpoint inhibitor therapy comprises inhibiting a protein selected from: PD-1, PD-L1, and CTLA-4, or a combination thereof.

24. The method of claim 23, wherein the immune checkpoint inhibitor therapy comprises inhibiting PD-1 and/or CTLA4.

25. (canceled)

26. The method of claim 1, wherein the immune checkpoint inhibitor is administered after the radiolabeled agent for binding to CAIX, preferably wherein the immune checkpoint inhibitor is administered at least 24 hours after administration of the radiolabeled agent for binding to CAIX.

27. (canceled)

28. The method of claim 1, wherein the cancer is renal cancer.

29.-34. (canceled)

35. A kit when used or for use according to the method of claim 1, the kit comprising in separate parts:
(A)
an agent that specifically binds to CAIX, optionally conjugated with a radionuclide; and
instructions for its use in combination with an immune checkpoint inhibitor;
optionally wherein the agent is an antibody or antigen binding fragment thereof for binding to CAIX, preferably wherein the antibody or antigen binding fragment thereof is a girentuximab antibody or antigen binding fragment thereof; and
optionally wherein the kit further comprises, in a separate part, one or more immune checkpoint inhibitor(s): or
(B)
one or more immune checkpoint inhibitors; and
instructions for its use in combination with an antibody or antigen binding fragment thereof for binding to CAIX conjugated with a radionuclide:
optionally wherein the kit further comprises, in a separate part, an agent, preferably an antibody or antigen binding fragment thereof, for binding to CAIX optionally conjugated with a radionuclide.

36.-39. (canceled)

40. The kit of any claim 35, wherein the agent for binding to CAIX and/or the immune checkpoint inhibitor are provided in the form of a pharmaceutical composition optionally comprising one or more pharmaceutically acceptable excipients, carriers and/or diluents.

41. The method of claim 1, wherein administrations of the radiolabelled agent and the immune checkpoint inhibitor are sequential in either order, at the same time, or staggered.

42. The method of claim 1, wherein the immune checkpoint inhibitor is administered prior to the radiolabelled agent.

43. The method of claim 13, wherein the antigen binding protein comprises the amino acid sequence of a VH and a VL as set forth in SEQ ID NO: 52/SEQ ID NO: 148.


SEQUENCE LISTING (186 sequences)

SEQ ID NO: 1 - protein, 5 aa, Synthetic construct
NYYMS

SEQ ID NO: 2 - protein, 17 aa, Synthetic construct
AINSDGGITYYLDTVKG

SEQ ID NO: 3 - protein, 10 aa, Synthetic construct
HRSGYFSMDY

SEQ ID NO: 4 - protein, 119 aa, Synthetic construct
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSS

SEQ ID NO: 5 - DNA, 15 bp, Synthetic construct
  source 1..15: mol_type = other DNA
aactattacatgtct

SEQ ID NO: 6 - DNA, 51 bp, Synthetic construct
  source 1..51: mol_type = other DNA
gccattaatagtgatggtggtatcacctactatctagacactgtgaagggc

SEQ ID NO: 7 - DNA, 30 bp, Synthetic construct
  source 1..30: mol_type = other DNA
caccgctcgggctacttttctatggactac

SEQ ID NO: 8 - DNA, 357 bp, Synthetic construct
  source 1..357: mol_type = other DNA
gacgtgaagctcgtggagtctgggggaggcttagtgaagcttggaggatccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctca

SEQ ID NO: 9 - protein, 30 aa, Synthetic construct
DVKLVESGGGLVKLGGSLKLSCAASGFTFS

SEQ ID NO: 10 - protein, 14 aa, Synthetic construct
WVRQTPEKRLELVA

SEQ ID NO: 11 - protein, 32 aa, Synthetic construct
RFTISRDNAKNTLYLQMSSLKSEDTALFYCAR

SEQ ID NO: 12 - protein, 11 aa, Synthetic construct
WGQGTSVTVSS

SEQ ID NO: 13 - DNA, 90 bp, Synthetic construct
  source 1..90: mol_type = other DNA
gacgtgaagctcgtggagtctgggggaggcttagtgaagcttggaggatccctgaaactctcctgtgcagcctctggattcactttcagt

SEQ ID NO: 14 - DNA, 42 bp, Synthetic construct
  source 1..42: mol_type = other DNA
tgggttcgccagactccagagaagaggctggagttggtcgca

SEQ ID NO: 15 - DNA, 96 bp, Synthetic construct
  source 1..96: mol_type = other DNA
cgattcaccatttcaagagacaatgccaagaacaccctgtacctgcaaatgagcagtctgaagtctgaggacacagccttgttttactgtgcaaga

SEQ ID NO: 16 - DNA, 33 bp, Synthetic construct
  source 1..33: mol_type = other DNA
tggggtcaaggaacctcagtcaccgtctcctca

SEQ ID NO: 17 - protein, 5 aa, Synthetic construct
NYYMS

SEQ ID NO: 18 - protein, 17 aa, Synthetic construct
AINSDGGITYYLDTVKG

SEQ ID NO: 19 - protein, 10 aa, Synthetic construct
HRSGYFSMDY

SEQ ID NO: 20 - protein, 119 aa, Synthetic construct
DVKLVESGGGLVKPGGSLRLSCAASGFTFSNYYMSWVRQAPGKGLELVAAINSDGGITYYLDTVKGRFTISRDNAKNTLYLQMSSLKSEDTALYYCARHRSGYFSMDYWGQGTSVTVSS

SEQ ID NO: 21 - DNA, 15 bp, Synthetic construct
  source 1..15: mol_type = other DNA
aactactacatgagc

SEQ ID NO: 22 - DNA, 51 bp, Synthetic construct
  source 1..51: mol_type = other DNA
gccattaacagtgacggtggcatcacctactacctggacaccgtgaagggc

SEQ ID NO: 23 - DNA, 30 bp, Synthetic construct
  source 1..30: mol_type = other DNA
cacaggagcggctacttctctatggactac

SEQ ID NO: 24 - DNA, 357 bp, Synthetic construct
  source 1..357: mol_type = other DNA
gacgtgaagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgagcagcctgaagagcgaggacacggccctgtattactgtgcgagacacaggagcggctacttctctatggactactggggccagggcaccagcgtcactgtctcctca

SEQ ID NO: 25 - protein, 30 aa, Synthetic construct
DVKLVESGGGLVKPGGSLRLSCAASGFTFS

SEQ ID NO: 26 - protein, 14 aa, Synthetic construct
WVRQAPGKGLELVA

SEQ ID NO: 27 - protein, 32 aa, Synthetic construct
RFTISRDNAKNTLYLQMSSLKSEDTALYYCAR

SEQ ID NO: 28 - protein, 11 aa, Synthetic construct
WGQGTSVTVSS

SEQ ID NO: 29 - DNA, 90 bp, Synthetic construct
  source 1..90: mol_type = other DNA
gacgtgaagctggtggagtctgggggaggcttggtcaagcctggaggatccctgagactctcctgtgcagcctctggattcaccttcagt

SEQ ID NO: 30 - DNA, 42 bp, Synthetic construct
  source 1..42: mol_type = other DNA
tgggtgcgccaggctccagggaaggggctggagctggttgcc

SEQ ID NO: 31 - DNA, 96 bp, Synthetic construct
  source 1..96: mol_type = other DNA
cgattcaccatctccagggacaacgccaagaacaccctgtatctgcaaatgagcagcctgaagagcgaggacacggccctgtattactgtgcgaga

SEQ ID NO: 32 - DNA, 33 bp, Synthetic construct
  source 1..33: mol_type = other DNA
tggggccagggcaccagcgtcactgtctcctca

SEQ ID NO: 33 - protein, 5 aa, Synthetic construct
NYYMS

SEQ ID NO: 34 - protein, 17 aa, Synthetic construct
AINSDGGITYYLDTVKG

SEQ ID NO: 35 - protein, 10 aa, Synthetic construct
HRSGYFSMDY

SEQ ID NO: 36 - protein, 119 aa, Synthetic construct
DVKLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSS

SEQ ID NO: 37 - DNA, 15 bp, Synthetic construct
  source 1..15: mol_type = other DNA
aactactacatgagc

SEQ ID NO: 38 - DNA, 50 bp, Synthetic construct
  source 1..50: mol_type = other DNA
ccattaacagtgacggtggcatcacctactacctggacaccgtgaagggc

SEQ ID NO: 39 - DNA, 30 bp, Synthetic construct; identical to SEQ ID NO: 23
  source 1..30: mol_type = other DNA

SEQ ID NO: 40 - DNA, 357 bp, Synthetic construct
  source 1..357: mol_type = other DNA
gacgtgaagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 41 - protein, 30 aa, Synthetic construct; identical to SEQ ID NO: 25

SEQ ID NO: 42 - protein, 14 aa, Synthetic construct
WVRQAPGKGLELVA

SEQ ID NO: 43 - protein, 32 aa, Synthetic construct
RFTISRDNAKNTLYLQMNSLRAEDTALYYCAR

SEQ ID NO: 44 - protein, 11 aa, Synthetic construct
WGQGTLVTVSS

SEQ ID NO: 45 - DNA, 90 bp, Synthetic construct; identical to SEQ ID NO: 29
  source 1..90: mol_type = other DNA

SEQ ID NO: 46 - DNA, 42 bp, Synthetic construct; identical to SEQ ID NO: 30
  source 1..42: mol_type = other DNA

SEQ ID NO: 47 - DNA, 96 bp, Synthetic construct
  source 1..96: mol_type = other DNA
cgattcaccatctccagggacaacgccaagaacaccctgtatctgcaaatgaacagcctgagggccgaggacacggccctgtattactgtgcgaga

SEQ ID NO: 48 - DNA, 33 bp, Synthetic construct
  source 1..33: mol_type = other DNA
tggggccagggcaccctggtcactgtctcctca

SEQ ID NO: 49 - protein, 5 aa, Synthetic construct
NYYMS

SEQ ID NO: 50 - protein, 34 aa, Synthetic construct
GIRENTUXIMABHUMANISEDVHHCDRPROTEIN

SEQ ID NO: 51 - protein, 10 aa, Synthetic construct
HRSGYFSMDY

SEQ ID NO: 52 - protein, 119 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSS

SEQ ID NO: 53 - DNA, 15 bp, Synthetic construct
  source 1..15: mol_type = other DNA
aactactacatgagc

SEQ ID NO: 54 - DNA, 51 bp, Synthetic construct; identical to SEQ ID NO: 22
  source 1..51: mol_type = other DNA

SEQ ID NO: 55 - DNA, 30 bp, Synthetic construct; identical to SEQ ID NO: 23
  source 1..30: mol_type = other DNA

SEQ ID NO: 56 - DNA, 357 bp, Synthetic construct
  source 1..357: mol_type = other DNA
gaggtgcagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 57 - protein, 30 aa, Synthetic construct
EVQLVESGGGLVKPGGSLRLSCAASGFTFS

SEQ ID NO: 58 - protein, 14 aa, Synthetic construct
WVRQAPGKGLELVA

SEQ ID NO: 59 - protein, 32 aa, Synthetic construct; identical to SEQ ID NO: 43

SEQ ID NO: 60 - protein, 11 aa, Synthetic construct
WGQGTLVTVSS

SEQ ID NO: 61 - DNA, 90 bp, Synthetic construct
  source 1..90: mol_type = other DNA
gaggtgcagctggtggagtctgggggaggcttggtcaagcctggaggatccctgagactctcctgtgcagcctctggattcaccttcagt

SEQ ID NO: 62 - DNA, 42 bp, Synthetic construct; identical to SEQ ID NO: 30
  source 1..42: mol_type = other DNA

SEQ ID NO: 63 - DNA, 96 bp, Synthetic construct; identical to SEQ ID NO: 47
  source 1..96: mol_type = other DNA

SEQ ID NO: 64 - DNA, 33 bp, Synthetic construct; identical to SEQ ID NO: 48
  source 1..33: mol_type = other DNA

SEQ ID NO: 65 - protein, 5 aa, Synthetic construct
NYYMS

SEQ ID NO: 66 - protein, 17 aa, Synthetic construct
AINSDGGITYYLDTVKG

SEQ ID NO: 67 - protein, 10 aa, Synthetic construct
HRSGYFSMDY

SEQ ID NO: 68 - protein, 119 aa, Synthetic construct
EVQLVESGGGLVKPGGSLRLSCAASGFTFSNYYMSWVRQAPGKGLEWVAAINSDGGITYYLDTVKGRFTISRDNAKNTLYLQMNSLRAEDTALYYCARHRSGYFSMDYWGQGTLVTVSS

SEQ ID NO: 69 - DNA, 15 bp, Synthetic construct
  source 1..15: mol_type = other DNA
aactactacatgagc

SEQ ID NO: 70 - DNA, 51 bp, Synthetic construct; identical to SEQ ID NO: 22
  source 1..51: mol_type = other DNA

SEQ ID NO: 71 - DNA, 30 bp, Synthetic construct; identical to SEQ ID NO: 23
  source 1..30: mol_type = other DNA

SEQ ID NO: 72 - DNA, 357 bp, Synthetic construct
  source 1..357: mol_type = other DNA
gaggtgcagctggtggagtctgggggaggcttggtcaagcctggaggatccctgagactctcctgtgcagcctctggattcaccttcagtaactactacatgagctgggtgcgccaggctccagggaaggggctggagtgggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 73 - protein, 30 aa, Synthetic construct; identical to SEQ ID NO: 57

SEQ ID NO: 74 - protein, 14 aa, Synthetic construct
WVRQAPGKGLEWVA

SEQ ID NO: 75 - protein, 32 aa, Synthetic construct; identical to SEQ ID NO: 43

SEQ ID NO: 76 - protein, 11 aa, Synthetic construct
WGQGTLVTVSS

SEQ ID NO: 77 - DNA, 90 bp, Synthetic construct; identical to SEQ ID NO: 61
  source 1..90: mol_type = other DNA

SEQ ID NO: 78 - DNA, 42 bp, Synthetic construct
  source 1..42: mol_type = other DNA
tgggtgcgccaggctccagggaaggggctggagtgggttgcc

SEQ ID NO: 79 - DNA, 96 bp, synthetic construct; identical to SEQ ID NO: 47
  source 1..96: mol_type = other DNA

SEQ ID NO: 80 - DNA, 33 bp, Synthetic construct; identical to SEQ ID NO: 48
  source 1..33: mol_type = other DNA

SEQ ID NO: 81 - protein, 11 aa, Synthetic construct
KASQNVVSAVA

SEQ ID NO: 82 - protein, 7 aa, Synthetic construct
SASNRYT

SEQ ID NO: 83 - protein, 9 aa, Synthetic construct
QQYSNYPWT

SEQ ID NO: 84 - protein, 107 aa, Synthetic construct
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIK

SEQ ID NO: 85 - DNA, 33 bp, Synthetic construct
  source 1..33: mol_type = other DNA
aaggccagtcagaatgtggtttctgctgttgcc

SEQ ID NO: 86 - DNA, 21 bp, Synthetic construct
  source 1..21: mol_type = other DNA
tcagcatccaatcggtacact

SEQ ID NO: 87 - DNA, 27 bp, Synthetic construct
  source 1..27: mol_type = other DNA
caacaatatagcaactatccgtggacg

SEQ ID NO: 88 - DNA, 321 bp, Synthetic construct
  source 1..321: mol_type = other DNA
gacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaa

SEQ ID NO: 89 - protein, 23 aa, Synthetic construct
DIVMTQSQRFMSTTVGDRVSITC

SEQ ID NO: 90 - protein, 15 aa, Synthetic construct
WYQQKPGQSPKLLIY

SEQ ID NO: 91 - protein, 32 aa, Synthetic construct
GVPDRFTGSGSGTDFTLTISNMQSEDLADFFC

SEQ ID NO: 92 - protein, 10 aa, Synthetic construct
FGGGTKLEIK

SEQ ID NO: 93 - DNA, 69 bp, Synthetic construct
  source 1..69: mol_type = other DNA
gacattgtgatgacccagtctcaaagattcatgtccacaacagtaggagacagggtcagcatcacctgc

SEQ ID NO: 94 - DNA, 45 bp, Synthetic construct
  source 1..45: mol_type = other DNA
tggtatcaacagaaaccaggacaatctcctaaactactgatttac

SEQ ID NO: 95 - DNA, 96 bp, Synthetic construct
  source 1..96: mol_type = other DNA
ggagtccctgatcgcttcacaggcagtggatctgggacagatttcactctcaccattagcaatatgcagtctgaagacctggctgattttttctgt

SEQ ID NO: 96 - DNA, 30 bp, Synthetic construct
  source 1..30: mol_type = other DNA
ttcggtggaggcaccaagctggaaatcaaa

SEQ ID NO: 97 - protein, 11 aa, Synthetic construct
KASQNVVSAVA

SEQ ID NO: 98 - protein, 7 aa, Synthetic construct
SASNRYT

SEQ ID NO: 99 - protein, 9 aa, Synthetic construct
QQYSNYPWT

SEQ ID NO: 100 - protein, 107 aa, Synthetic construct
DIVMTQSPSFLSASVGDRVTITCKASQNVVSAVAWYQQKPGQAPKLLIYSASNRYTGVPDRFTGSGSGTDFTLTISSLQSEDLADYFCQQYSNYPWTFGGGTKVEIK

SEQ ID NO: 101 - DNA, 33 bp, Synthetic construct
  source 1..33: mol_type = other DNA
aaggcaagtcagaacgtggtgagtgctgtggcc

SEQ ID NO: 102 - DNA, 21 bp, Synthetic construct
  source 1..21: mol_type = other DNA
agcgcctccaacaggtacacc

SEQ ID NO: 103 - DNA, 27 bp, Synthetic construct
  source 1..27: mol_type = other DNA
caacagtacagcaattacccttggacg

SEQ ID NO: 104 - DNA, 321 bp, Synthetic construct
  source 1..321: mol_type = other DNA
gacatcgtgatgacccagtctccatccttcctgtctgcatctgtaggagacagagtcaccatcacttgcaaggcaagtcagaacgtggtgagtgctgtggcctggtatcagcagaaaccagggcaggctcctaagctcctgatctatagcgcctccaacaggtacaccggggtcccagacaggttcaccggcagtggatctgggacagatttcactctcaccatcagcagcctgcagagcgaagatctggcagactatttctgtcaacagtacagcaattacccttggacgttcggcggcgggaccaaggtggaaatcaaa

SEQ ID NO: 105 - protein, 23 aa, Synthetic construct
DIVMTQSPSFLSASVGDRVTITC

SEQ ID NO: 106 - protein, 15 aa, Synthetic construct
WYQQKPGQAPKLLIY

SEQ ID NO: 107 - protein, 32 aa, Synthetic construct
GVPDRFTGSGSGTDFTLTISSLQSEDLADYFC

SEQ ID NO: 108 - protein, 10 aa, Synthetic construct
FGGGTKVEIK

SEQ ID NO: 109 - DNA, 69 bp, Synthetic construct
  source 1..69: mol_type = other DNA
gacatcgtgatgacccagtctccatccttcctgtctgcatctgtaggagacagagtcaccatcacttgc

SEQ ID NO: 110 - DNA, 45 bp, Synthetic construct
  source 1..45: mol_type = other DNA
tggtatcagcagaaaccagggcaggctcctaagctcctgatctat

SEQ ID NO: 111 - DNA, 96 bp, Synthetic construct
  source 1..96: mol_type = other DNA
ggggtcccagacaggttcaccggcagtggatctgggacagatttcactctcaccatcagcagcctgcagagcgaagatctggcagactatttctgt

SEQ ID NO: 112 - DNA, 30 bp, Synthetic construct
  source 1..30: mol_type = other DNA
ttcggcggcgggaccaaggtggaaatcaaa

SEQ ID NO: 113 - protein, 11 aa, Synthetic construct
KASQNVVSAVA

SEQ ID NO: 114 - protein, 7 aa, Synthetic construct
SASNRYT

SEQ ID NO: 115 - protein, 9 aa, Synthetic construct
QQYSNYPWT

SEQ ID NO: 116 - protein, 107 aa, Synthetic construct
DIVMTQSPSSLSASVGDRVTITCKASQNVVSAVAWYQQKPGQAPRLLIYSASNRYTGVPDRFTGSGSGTDFTLTISSLQAEDLADYFCQQYSNYPWTFGGGTKVEIK

SEQ ID NO: 117 - DNA, 33 bp, Synthetic construct; identical to SEQ ID NO: 101
  source 1..33: mol_type = other DNA

SEQ ID NO: 118 - DNA, 21 bp, Synthetic construct
  source 1..21: mol_type = other DNA
agcgcctccaacaggtacacc

SEQ ID NO: 119 - DNA, 27 bp, Synthetic construct
  source 1..27: mol_type = other DNA
caacagtacagcaattacccttggacg

SEQ ID NO: 120 - DNA, 321 bp, Synthetic construct
  source 1..321: mol_type = other DNA
gacatcgtgatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcaccggcagtggatctgggacagatttcactctcaccatcagcagcctgcaggccgaagatctggcagactatttctgtcaacagtacagcaattacccttggacgttcggcggcgggaccaaggtggaaatcaaa

SEQ ID NO: 121 - protein, 23 aa, Synthetic construct
DIVMTQSPSSLSASVGDRVTITC

SEQ ID NO: 122 - protein, 15 aa, Synthetic construct
WYQQKPGQAPRLLIY

SEQ ID NO: 123 - protein, 32 aa, Synthetic construct
GVPDRFTGSGSGTDFTLTISSLQAEDLADYFC

SEQ ID NO: 124 - protein, 10 aa, Synthetic construct
FGGGTKVEIK

SEQ ID NO: 125 - DNA, 69 bp, Synthetic construct
  source 1..69: mol_type = other DNA
gacatcgtgatgacccagtctccatccagcctgtctgcatctgtaggagacagagtcaccatcacttgc

SEQ ID NO: 126 - DNA, 45 bp, Synthetic construct
  source 1..45: mol_type = other DNA
tggtatcagcagaaaccagggcaggctcctaggctcctgatctat

SEQ ID NO: 127 - DNA, 96 bp, Synthetic construct
  source 1..96: mol_type = other DNA
ggggtcccagacaggttcaccggcagtggatctgggacagatttcactctcaccatcagcagcctgcaggccgaagatctggcagactatttctgt

SEQ ID NO: 128 - DNA, 30 bp, Synthetic construct; identical to SEQ ID NO: 112
  source 1..30: mol_type = other DNA

SEQ ID NO: 129 - protein, 11 aa, Synthetic construct
KASQNVVSAVA

SEQ ID NO: 130 - protein, 7 aa, Synthetic construct
SASNRYT

SEQ ID NO: 131 - protein, 9 aa, Synthetic construct
QQYSNYPWT

SEQ ID NO: 132 - protein, 107 aa, Synthetic construct
DIQMTQSPSSLSASVGDRVTITCKASQNVVSAVAWYQQKPGQAPRLLIYSASNRYTGVPDRFSGSGSGTDFTLTISSLQAEDLADYFCQQYSNYPWTFGGGTKVEIK

SEQ ID NO: 133 - DNA, 33 bp, Synthetic construct; identical to SEQ ID NO: 101
  source 1..33: mol_type = other DNA

SEQ ID NO: 134 - DNA, 21 bp, Synthetic construct
  source 1..21: mol_type = other DNA
agcgcctccaacaggtacacc

SEQ ID NO: 135 - DNA, 27 bp, Synthetic construct
  source 1..27: mol_type = other DNA
caacagtacagcaattacccttggacg

SEQ ID NO: 136 - DNA, 321 bp, Synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcagcggcagtggatctgggacagatttcactctcaccatcagcagcctgcaggccgaagatctggcagactatttctgtcaacagtacagcaattacccttggacgttcggcggcgggaccaaggtggaaatcaaa

SEQ ID NO: 137 - protein, 23 aa, Synthetic construct
DIQMTQSPSSLSASVGDRVTITC

SEQ ID NO: 138 - protein, 15 aa, Synthetic construct
WYQQKPGQAPRLLIY

SEQ ID NO: 139 - protein, 32 aa, Synthetic construct
GVPDRFSGSGSGTDFTLTISSLQAEDLADYFC

SEQ ID NO: 140 - protein, 10 aa, Synthetic construct
FGGGTKVEIK

SEQ ID NO: 141 - DNA, 69 bp, Synthetic construct
  source 1..69: mol_type = other DNA
gacatccagatgacccagtctccatccagcctgtctgcatctgtaggagacagagtcaccatcacttgc

SEQ ID NO: 142 - DNA, 45 bp, Synthetic construct; identical to SEQ ID NO: 126
  source 1..45: mol_type = other DNA

SEQ ID NO: 143 - DNA, 96 bp, Synthetic construct
  source 1..96: mol_type = other DNA
ggggtcccagacaggttcagcggcagtggatctgggacagatttcactctcaccatcagcagcctgcaggccgaagatctggcagactatttctgt

SEQ ID NO: 144 - DNA, 30 bp, Synthetic construct; identical to SEQ ID NO: 112
  source 1..30: mol_type = other DNA

SEQ ID NO: 145 - protein, 11 aa, Synthetic construct
KASQNVVSAVA

SEQ ID NO: 146 - protein, 7 aa, Synthetic construct
SASNRYT

SEQ ID NO: 147 - protein, 9 aa, Synthetic construct
QQYSNYPWT

SEQ ID NO: 148 - protein, 107 aa, Synthetic construct
DIQMTQSPSSLSASVGDRVTITCKASQNVVSAVAWYQQKPGQAPRLLIYSASNRYTGVPDRFSGSGSGTDFTLTISSLQAEDLADYYCQQYSNYPWTFGGGTKVEIK

SEQ ID NO: 149 - DNA, 33 bp, Synthetic construct; identical to SEQ ID NO: 101
  source 1..33: mol_type = other DNA

SEQ ID NO: 150 - DNA, 21 bp, Synthetic construct
  source 1..21: mol_type = other DNA
agcgcctccaacaggtacacc

SEQ ID NO: 151 - DNA, 27 bp, Synthetic construct
  source 1..27: mol_type = other DNA
caacagtacagcaattacccttggacg

SEQ ID NO: 152 - DNA, 321 bp, Synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcagcggcagtggatctgggacagatttcactctcaccatcagcagcctgcaggccgaagatctggcagactattactgtcaacagtacagcaattacccttggacgttcggcggcgggaccaaggtggaaatcaaa

SEQ ID NO: 153 - protein, 23 aa, Synthetic construct
DIQMTQSPSSLSASVGDRVTITC

SEQ ID NO: 154 - protein, 15 aa, Synthetic construct
WYQQKPGQAPRLLIY

SEQ ID NO: 155 - protein, 32 aa, Synthetic construct
GVPDRFSGSGSGTDFTLTISSLQAEDLADYYC

SEQ ID NO: 156 - protein, 10 aa, Synthetic construct
FGGGTKVEIK

SEQ ID NO: 157 - DNA, 69 bp, Synthetic construct; identical to SEQ ID NO: 141
  source 1..69: mol_type = other DNA

SEQ ID NO: 158 - DNA, 45 bp, Synthetic construct; identical to SEQ ID NO: 126
  source 1..45: mol_type = other DNA

SEQ ID NO: 159 - DNA, 96 bp, Synthetic construct
  source 1..96: mol_type = other DNA
ggggtcccagacaggttcagcggcagtggatctgggacagatttcactctcaccatcagcagcctgcaggccgaagatctggcagactattactgt

SEQ ID NO: 160 - DNA, 30 bp, Synthetic construct; identical to SEQ ID NO: 112
  source 1..30: mol_type = other DNA

SEQ ID NO: 161 - protein, 11 aa, Synthetic construct
KASQNVVSAVA

SEQ ID NO: 162 - protein, 7 aa, Synthetic construct
SASNRYT

SEQ ID NO: 163 - protein, 9 aa, Synthetic construct
QQYSNYPWT

SEQ ID NO: 164 - protein, 107 aa, Synthetic construct
DIQMTQSPSSLSASVGDRVTITCKASQNVVSAVAWYQQKPGQAPRRLIYSASNRYTGVPDRFSGSGSGTDFTLTISSLQAEDLADYYCQQYSNYPWTFGGGTKVEIK

SEQ ID NO: 165 - DNA, 33 bp, Synthetic construct; identical to SEQ ID NO: 101
  source 1..33: mol_type = other DNA

SEQ ID NO: 166 - DNA, 21 bp, Synthetic construct
  source 1..21: mol_type = other DNA
agcgcctccaacaggtacacc

SEQ ID NO: 167 - DNA, 27 bp, Synthetic construct
  source 1..27: mol_type = other DNA
caacagtacagcaattacccttggacg

SEQ ID NO: 168 - DNA, 321 bp, Synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggaggctgatctatagcgcctccaacaggtacaccggggtcccagacaggttcagcggcagtggatctgggacagatttcactctcaccatcagcagcctgcaggccgaagatctggcagactattactgtcaacagtacagcaattacccttggacgttcggcggcgggaccaaggtggaaatcaaa

SEQ ID NO: 169 - protein, 23 aa, Synthetic construct
DIQMTQSPSSLSASVGDRVTITC

SEQ ID NO: 170 - protein, 32 aa, Synthetic construct
GIRENTUXIMABCHIMERICVKLFRPROTEIN

SEQ ID NO: 171 - protein, 32 aa, Synthetic construct; identical to SEQ ID NO: 155

SEQ ID NO: 172 - protein, 10 aa, Synthetic construct
FGGGTKVEIK

SEQ ID NO: 173 - protein, 69 aa, Synthetic construct
GACATCCAGATGACCCAGTCTCCATCCAGCCTGTCTGCATCTGTAGGAGACAGAGTCACCATCACTTGC

SEQ ID NO: 174 - protein, 45 aa, Synthetic construct
TGGTATCAGCAGAAACCAGGGCAGGCTCCTAGGAGGCTGATCTAT

SEQ ID NO: 175 - protein, 96 aa, Synthetic construct
GGGGTCCCAGACAGGTTCAGCGGCAGTGGATCTGGGACAGATTTCACTCTCACCATCAGCAGCCTGCAGGCCGAAGATCTGGCAGACTATTACTGT

SEQ ID NO: 176 - protein, 30 aa, synthetic construct
TTCGGCGGCGGGACCAAGGTGGAAATCAAA

SEQ ID NO: 177 - protein, 330 aa, Synthetic construct [sequence omitted]

SEQ ID NO: 178 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 179 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 180 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 181 - protein, 107 aa, Synthetic construct [sequence omitted]

SEQ ID NO: 182 - protein, 449 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 449 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 184 - protein, 446 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 185 - protein, 446 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 186 - protein, 214 aa, Synthetic construct
DIQMTQSPSSLSASVGDRVTITCKASQNVVSAVAWYQQKPGQAPRLLIYSASNRYTGVPDRFSGSGSGTDFTLTISSLQAEDLADYYCQQYSNYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC